(19) United States
(12) Patent Application Publication
(10) Pub. No.: US 2009/0182189 A1
(43) Pub. Date: Jul. 16, 2009

(54) Architectural Strategies to obtain light characteristics appropriate for human circadian stimulation

(75) Inventor: Adriana Lira, NewYork, NY (US)

Correspondence Address: FOLEY HOAG, LLP;PATENT GROUP (w/HUV HMV), 155 SEAPORT BLVD., BOSTON, MA, 02210-2600, US

(73) Assignee: President and Fellows of Harvard College, Cambridge, MA (US)

(21) Appl. No.: 12/291,154

(22) Filed: Nov. 6, 2008

Related U.S. Application Data
(60) Provisional application No. 60/985,829, filed on Nov. 6, 2007.

(51) Int. Cl. A61M 21/02

(52) U.S. Cl. 600/27

(57) ABSTRACT

One aspect of the invention relates to a method of stimulating the circadian system of a subject comprising the step of reflecting light off of a surface towards the subject.

### RELATED APPLICATIONS

This application claims the benefit of priority to U.S. Provisional Patent Application Ser. No. 60/985,829, filed Nov. 6, 2007; which is hereby incorporated by reference in its entirety.

### BACKGROUND

Visible light entering the eye not only enables sight, but also affects many psycho-physical human processes regulated by the circadian system: our “biological clock.” The specific light characteristics, such as spectrum, intensity, and spatial distribution that stimulate the human visual system are very different form those needed to stimulate the circadian system. See, for example, Bodmann, H. W. (1967). Quality of Interior Lighting based on Luminance. Transactions of the IES, 3, 22; Cockram, A. H., Collins, J. J, & Langdon, F. J. (1970). A Study for User Preferences for Fluorescent Lamp Colours for Daylighting and Night-time Lighting. Lighting Research & Technology, 2, 249; Comission Internationale de l'Ecleritage (CIE) (2004). Ocular Lighting Effects on Human Physiology and Behavior. Technical Report, CIE 158:2004; Davis R. G., & Ginthner, D. N. (1990). Correlated Color Temperature, Illuminance Level, and the Kruithof Curve. Journal of Illuminating Engineering Society, 19, 27; Kruithof, A. A. (1941). Tabular luminescence lamps for general illumination. Philips Technical Review, 6, 65 (FIG. 1.1.1A depicts Kruithof's Comfort Curve); Rea, Mark S., ed. (2000). IESNA Lighting Handbook: Reference and Application. New York, N.Y.: Illuminating Engineering Society of North America; and Rea, Mark S., (2002). Light—Much More than Vision. In Rensselaer Polytechnic Institute. Proceedings from the 5th Lighting Research Office, Lighting Research Symposium. Troy, N.Y.

The characteristics of light that stimulate both the visual and circadian systems are present in natural light, but indoor illumination systems have historically been designed to provide visual clarity only. The discovery of electricity in the 18th century and the invention of the light bulb in the 19th century have forever changed people's daily habits. The percentage of time that we spend indoors only increased over the last century, and thanks to artificial lighting and other interior environmental controls we can continue our daily activities long after sunset. Many advances have been made over the last century to improve the visual clarity provided by artificial illumination systems: spectrum and light intensity, as well as the color and brightness of materials against which light reflects, have long been subjects of study for the improvement of contrast luminance and the reduction of glare. However, such improvements to the visual clarity of interior environments do not necessarily enhance stimulation of the circadian system, because different characteristics of light are necessary for both biological systems to work optimally.

The circadian system is regulated by the alternation of light and dark conditions within the daily cycle of day and night. Even during the day, light changes in spectrum and intensity: during the early mornings and late afternoons natural light is richer in reds and less bright, while it is richer in blues and brighter around noon. Bright, blue light informs our body via the circadian system that it is day time while dim, red light, suggests that night is approaching. Many human biological rhythms are regulated by this light information: during night-dark conditions, our performance and alertness decrease until we fall asleep, and during day-light conditions, we feel awake as our performance and alertness increase.

If typical applications of artificial lighting in architectural settings fail adequately to stimulate the human circadian system, then the performance of our biological clock suffers, resulting in psycho-physical disorders and other health problems. Current biological research into the circadian system asks us, therefore, to re-think the design of indoor illumination systems in order to deliver light to the eye that would give the right information to our biological clock. Otherwise certain health problems related to inappropriate light quality will continue, and along with them low alertness and performance. This lack of proper circadian stimulation can result in poor intellectual performance, low alertness levels, psychological depression, and sometimes even cancer. Neurological experts in circadian systems, as well as illumination authorities such as Illuminating Engineering Society of North America (IESNA) and Comission Internationale d'Ecleirage (CIE) recognize that new discoveries about the non-visual effects of light in humans may provide the basis for major changes in future architectural lighting strategies.

### SUMMARY

One aspect of the invention relates to novel architectural strategies that obtain light characteristics appropriate for human circadian stimulation and optimize the effects of light's interaction with interior architectural surfaces as it relates to human psycho-physical behaviors. In certain embodiments, the strategies take advantage of the fact that certain materials (such as metals and opaque non-metals) have different optical qualities with light. In certain embodiments, by relating the relationship between architectural, material, and light parameters, optimal light for the efficient regulation of the human biological clock is delivered. Certain embodiments of the invention comprise a wall surface which reflects light of a specific spectrum and intensity towards a subject's work/rest environment for the efficient regulation of the subject's biological clock.

One aspect of the invention relates to a method of stimulating the circadian system of a subject comprising the step of reflecting light off of a surface towards the subject.

In certain embodiments, the present invention relates to the aforementioned method, wherein said light is non-monochromatic.

In certain embodiments, the present invention relates to the aforementioned method, wherein said light comprises blue light.

In certain embodiments, the present invention relates to the aforementioned method, wherein said light comprises light having a wavelength between about 460 nm and about 480 nm.

In certain embodiments, the present invention relates to the aforementioned method, wherein the radiant intensity of the light is between about 2,000 W/sr and about 5,000 W/sr.

In certain embodiments, the present invention relates to the aforementioned method, wherein the radiant intensity of the light is between about 3,000 W/sr and about 4,000 W/sr.

In certain embodiments, the present invention relates to the aforementioned method, wherein the radiant intensity of the light is about 3,500 W/sr.

In certain embodiments, the present invention relates to the aforementioned method, wherein the red radiant intensity is about 1,000 W/sr; the green radiant intensity is about 1,000 W/sr; and the blue radiant intensity is about 1,500 W/sr.

In certain embodiments, the present invention relates to the aforementioned method, wherein the RGB reflectance of the surface is about 0.1 (red), about 0.3 (green) and about 0.6 (blue).

In certain embodiments, the present invention relates to the aforementioned method, wherein the RGB reflectance of the surface is about 0.5 (red), about 0.5 (green) and about 0.5 (blue).

In certain embodiments, the present invention relates to the aforementioned method, wherein the RGB reflectance of the surface is about 0.6 (red), about 0.3 (green) and about 0.1 (blue).

In certain embodiments, the present invention relates to the aforementioned method, wherein the specularity of the surface is between about 0.01 and about 0.09.

In certain embodiments, the present invention relates to the aforementioned method, wherein the specularity of the surface is about 0.02.

In certain embodiments, the present invention relates to the aforementioned method, wherein the specularity of the surface is about 0.04.

In certain embodiments, the present invention relates to the aforementioned method, wherein the specularity of the surface is about 0.06.

In certain embodiments, the present invention relates to the aforementioned method, wherein the specularity of the surface is about 0.08.

In certain embodiments, the present invention relates to the aforementioned method, where the roughness of the surface is between about 0.01 and 0.20.

In certain embodiments, the present invention relates to the aforementioned method, where the roughness of the surface is about 0.01.

In certain embodiments, the present invention relates to the aforementioned method, where the roughness of the surface is about 0.1.

In certain embodiments, the present invention relates to the aforementioned method, where the roughness of the surface is about 0.2.

In certain embodiments, the present invention relates to the aforementioned method, wherein the surface comprises a non-metallic opaque insulator.

In certain embodiments, the present invention relates to the aforementioned method, wherein the surface comprises a plastic.

In certain embodiments, the present invention relates to the aforementioned method, wherein the surface comprises a metal.

In certain embodiments, the present invention relates to the aforementioned method, wherein the surface is substantially flat.

In certain embodiments, the present invention relates to the aforementioned method, wherein the surface is curved.

In certain embodiments, the present invention relates to the aforementioned method, wherein the surface is part of a wall.

In certain embodiments, the present invention relates to the aforementioned method, wherein the distance of the light from the surface is about 1 m.

In certain embodiments, the present invention relates to the aforementioned method, wherein the distance of the light from the surface is about 2 m.

In certain embodiments, the present invention relates to the aforementioned method, wherein the distance of the light from the surface is about 3 m.

In certain embodiments, the present invention relates to the aforementioned method, wherein the distance of the light from the surface is about 4 m.

In certain embodiments, the present invention relates to the aforementioned method, wherein the distance of the light from the surface is about 5 m.

In certain embodiments, the present invention relates to the aforementioned method, wherein the stimulation leads to improved performance, alertness, mood, or sense of well being of a subject.

The strategies proposed herein are the first of their kind to address this problem via design. These strategies enable architecture to actively respond to quality of life and well-being issues by fine-tuning the relationships between light, surface and space. In certain embodiments, the technology disclosed herein will also enable architecture to actively respond to quality of life and well-being issues by fine tuning the relationships between light, surface and space.

Remarkably, the technology disclosed herein will have significant implications for the improvement of performance, alertness, mood and well-being of individuals in their homes, offices, schools and hospitals. It will also lead to the development of new architectural applications for interior lighting systems and surface designs, including floors, walls, ceilings, and furniture. In certain embodiments, the technology is an entirely new approach to interior design and will improve the quality of life for people in their living and working environments.

### BRIEF DESCRIPTION OF THE FIGURES

FIG. 1.1.1A depicts Kruithof's Comfort Curve.

FIG. 2.1A depicts different circadian cycles.

FIG. 2.1B depicts a circadian cycle.

FIG. 2.1C depicts free running of TAU.

FIG. 2.1D depicts the location of suprachiasmatic nucleus.

FIG. 2.3A depicts circadian and visual neuronal paths.

FIG. 2.4A depicts the relation of melatonin production to seasons.

FIG. 2.5A depicts phase shifts in circadian rhythms.

FIG. 2.5.1.2A depicts the change in light exposure in modern times.

FIG. 2.5.1.2B depicts a portable dosimeter.

FIG. 2.5.1.2C depicts outdoor lighting conditions.

FIG. 2.6A depicts the suppression of melatonin with bright light.

FIG. 2.6.1.1A depicts bright blue light exposure.

FIG. 3.1A depicts light characteristics of the visual and circadian systems.

FIG. 3.1.2A depicts visual and circadian photoreceptors.

FIG. 3.1.2B depicts circadian and visual stimuli in the brain.

FIG. 3.1.2C depicts spatial distribution of light in a room.

FIG. 3.1.2D depicts time exposure of light stimulus. The yellow symbol represents the light pulse (light stimulus). There are certain times when the light stimulus does not result in a phase delay nor a phase advance (a). Light stimulus applied in the first half of the night results in a phase (b and c). Light stimulus applied in the second half of the night results in a phase advance (d and e).

FIG. 3.1.2E depicts photopic, scotopic, and line sensitivity curves.

FIG. 3.2.1A depicts a table of fundamental radiometric quantities.

FIG. 3.2.2A depicts irradiance and photopic illumination.

FIG. 3.2.2B depicts a table of fundamental photometric units.

FIG. 3.2.3A depicts a comparison of photopic and circadian sensitivity curves.

FIG. 3.3A depicts a table of light characteristics to support vision and circadian functions.

FIG. 4.1.1A depicts a description of light waves in space.

FIG. 4.1.1B depicts the visible spectrum.

FIG. 4.2A depicts an atomic composition of matter.

FIG. 4.3.1A depicts the energy levels in an atom.

FIG. 4.3.1B energy bands.

FIG. 4.3.2.1A depicts energy bands in insulators.

FIG. 4.3.2.2A depicts energy bands in metals.

FIG. 4.3.2.3A depicts energy bands in semiconductors.

FIG. 4.3.3.1A depicts light interaction with transparent insulators.

FIG. 4.3.3.1B depicts light interaction with translucent or opaque insulators.

FIG. 4.3.3.2A depicts light interaction with metals.

FIG. 4.4A depicts microscopic interaction between light and matter.

FIG. 4.4.1.1A depicts reflection of light on a surface.

FIG. 4.4.1.1B depicts diffuse, specular, and diffuse-specular reflection.

FIG. 4.4.1.1C depicts a phase change given by reflection.

FIG. 4.4.2.1A depicts spectral characteristics of the surface of glossy opaque insulators.

FIG. 4.4.2.1B depicts spectral characteristics of the surface of matte opaque insulators.

FIG. 4.4.2.2A depicts spectral characteristics of the surface of metals.

FIG. 4.4.2.2B depicts reflection color in metals and insulators under different colored illumination.

FIG. 5.1A depicts a comparison of a RADIANCE rendering and a photo.

FIG. 5.1B depicts an experimental comparison between radiance calculations and real measurements under daylight conditions (March 1995).

FIG. 5.3.2A depicts different RADIANCE values of specularity and roughness.

FIG. 6A depicts parameters of Brainard's experiment.

FIG. 6.1.1A depicts dimensions and configurations of simulated room.

FIG. 6.1.2A depicts parameters of the simulation.

FIG. 6.1.2.4A depicts description of the texture of the “north” wall.

FIG. 6.2A depicts a diagram of simulated cases.

FIG. 6.2B depicts a table of values of the parameters used in each simulation.

FIG. 7.1.2A depicts reflection of light from the insulator wall.

FIG. 7.1.2B depicts reflection of light from the metallic wall.

Figure A.1 depicts plane xy, plane xz, and plane zy described by sections passing through the focal point of the room.

Figure A.2 depicts the intensity of the RGB components of the spectrum of the reflected wall at the focal point of the room with a white wall.

Figure A.3 depicts the intensity of the RGB components of the spectrum of the reflected wall at the focal point of the room with a reddish wall.

Figure A.4 depicts the intensity of the RGB components of the spectrum of the reflected wall at the focal point of the room with a bluish wall.

Figure B.1 depicts graphs representing the variation of the blue spectrum reflected by different reflectances in relationship with roughness and specularity changes when R is 0.5, G is 0.5, and B is 0.5.

Figure B.2 depicts graphs representing the variation of the blue spectrum reflected by different reflectances in relationship with roughness and specularity changes when R is 0.6, G is 0.3, and B is 0.1.

Figure B.3 depicts graphs representing the variation of the blue spectrum reflected by different reflectances in relationship with roughness and specularity changes when R is 0.1, G is 0.3, and B is 0.6.

Figure B.4 depicts graphs representing the variation of the red spectrum reflected by different reflectances in relationship with roughness and specularity changes when R is 0.5, G is 0.5, and B is 0.5.

Figure B.5 depicts graphs representing the variation of the red spectrum reflected by different reflectances in relationship with roughness and specularity changes when R is 0.6, G is 0.3, and B is 0.1.

Figure B.6 depicts graphs representing the variation of the red spectrum reflected by different reflectances in relationship with roughness and specularity changes when R is 0.1, G is 0.3, and B is 0.6.

### DETAILED DESCRIPTION

One aspect of the invention consists in the formulation of architectural strategies to optimize the effects of light's interaction with interior architectural surfaces as it relates to human psycho-physical behaviors. Recent research within the field of biology reveals that visible light not only enables sight, but also regulates many psycho-physical human processes via the circadian-biological clock. The lack of proper circadian stimulation can result in poor intellectual performance, low alertness levels, psychological depression, and sometimes even cancer. Artificial light in indoor spaces, unlike natural light, does not include all the characteristics to stimulate both the visual and the circadian systems. To date, lighting systems development tends to be aimed at enhancing visual clarity alone. Consequently, interior spaces are currently not designed to optimally regulate human performance, mood, and alertness. These proposed strategies are the first of its kind to address this problem via design. It will enable architecture to actively respond to quality of life and wellbeing issues by fine-tuning the relationships between light, surface and space. One aspect of the invention relates to augmenting or changing artificial light so that it stimulates both the visual and circadian systems.

Certain strategies disclosed herein are a re-conceptualization of interior lighting in architectural environments from a singular concern with visual clarity to a more robust concept of light, material, and space whose characteristics can be calibrated to positively stimulate both the human visual and circadian systems. For example, certain embodiments of the invention relate to the interaction of materials which have different optical qualities and textures (such as metals and opaque non-metals) with light thereby, thereby allowing strategic and passive spatial control of both the spectrum and the intensity of indoor illumination.

Certain aspects of the invention relate to simulated experiments. For example, as described in the Exemplification below, a computer lighting simulation of a cubic room was performed in order calculate the spectrum and intensity of light, at a specific point in space, while changing material optical properties and texture.

One aspect of the invention relates to a wall surface reflects light with a specific spectrum-intensity towards a subject's work/rest environment. With such a wall surface one would be able to measure the resultant mood-performance-alertness responses in subjects. By so doing, one could determine the optimal light needed for the efficient regulation of the human biological clock. Such a determination will involve analysis of the relationship between architectural, material, and light parameters: surface dimensions, textures, and geometry; optical material properties; and spectral-intensity light characteristics.

Certain aspects of the invention relate to the use of light and material simulation software, as well as the study of physical prototypes, to analyze the behavior of light and its spectral and intensity characteristics after it interacts with certain optical material properties, surface textures and surface geometries.

Other aspects of the invention relate to a wall surface. In certain embodiments, the wall surface is approximately 10 feet by 10 feet. In certain embodiments, the wall surface reflects light with a specific spectrum and intensity towards a subject's work/rest environment.

It yet other embodiments, the invention relates to the physical testing of the behavior of the light after interacting with the wall surface, and the analysis of mood-performance-alertness responses in subjects before, during, and after being exposed to lighting reflected off of the constructed wall surface.

For the reasons outlined above, as well as those discussed elsewhere herein, certain aspects of the invention have significant implications for the improvement of performance, alertness, mood and wellbeing of individuals in their homes, offices, schools and hospitals. For example, certain aspects of the invention relate to new architectural applications for interior lighting systems and surface designs, including floors, walls, ceilings, and furniture. Certain aspects of the invention may also relate to new lighting design guidelines and regulations, the need for which has already been expressed by lighting authorities such as the IESNA and CIE.

### The Human Circadian System

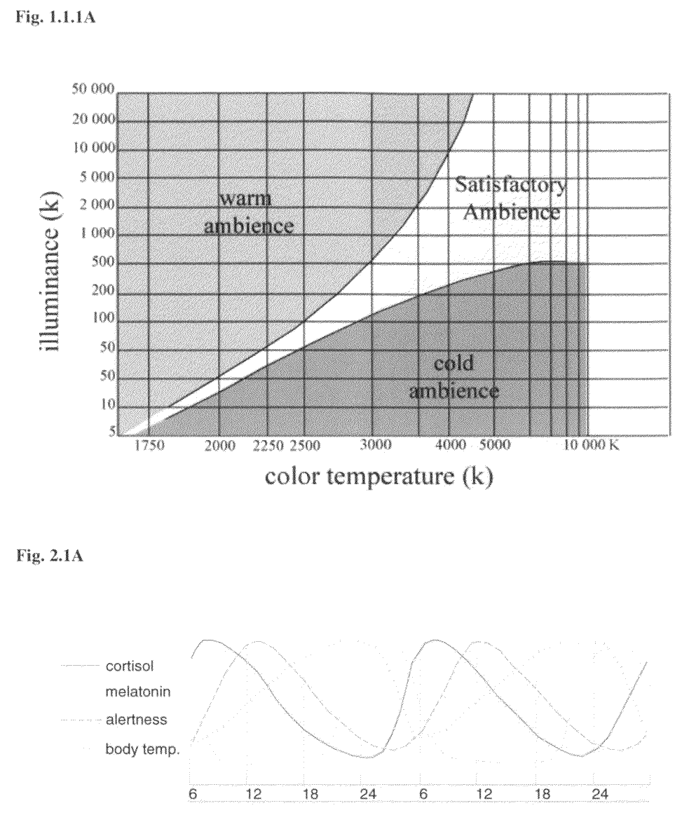

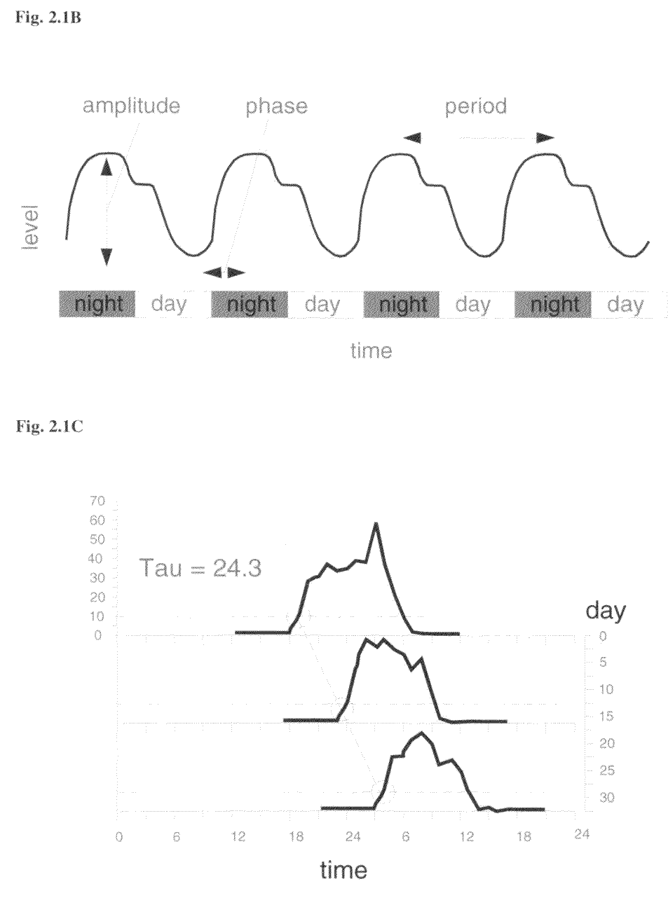

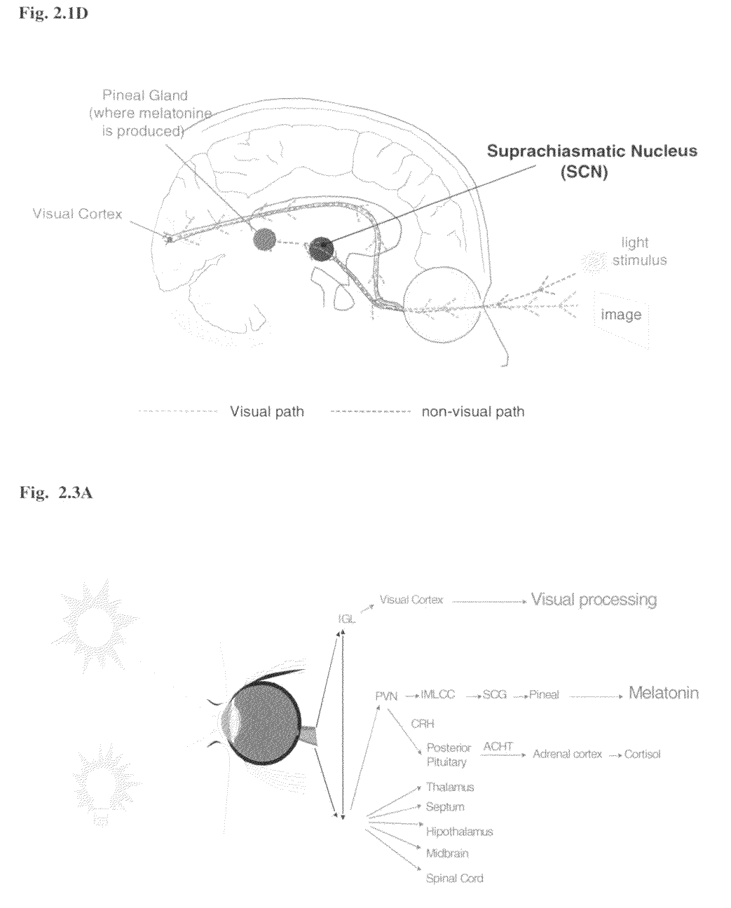

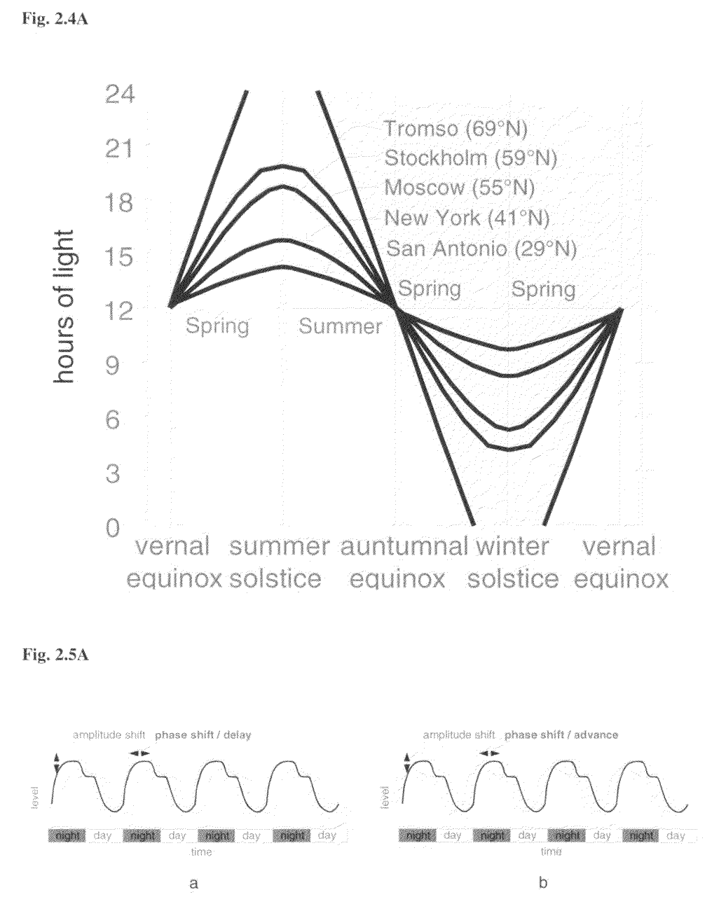

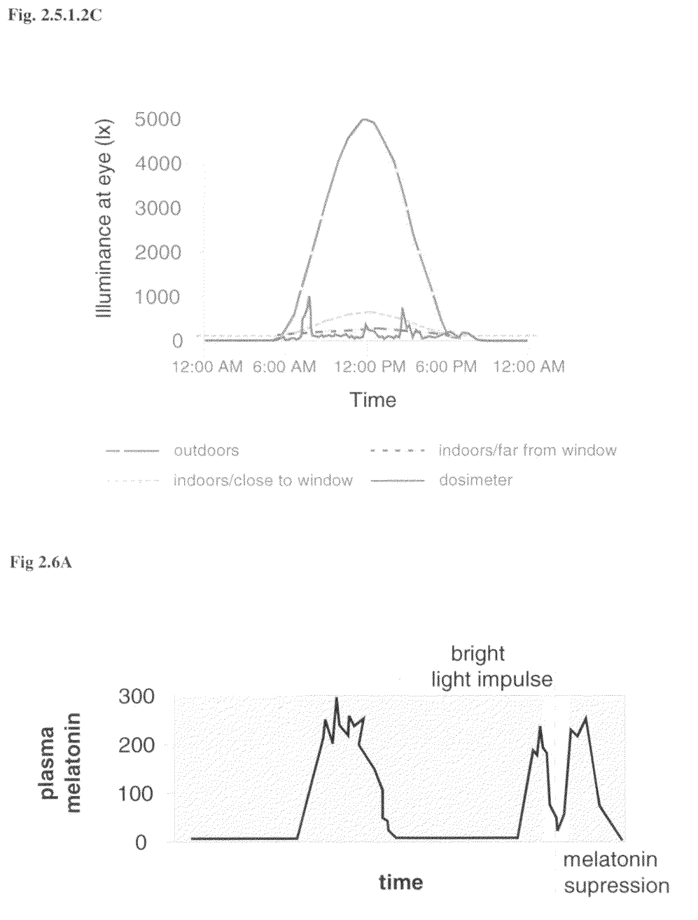

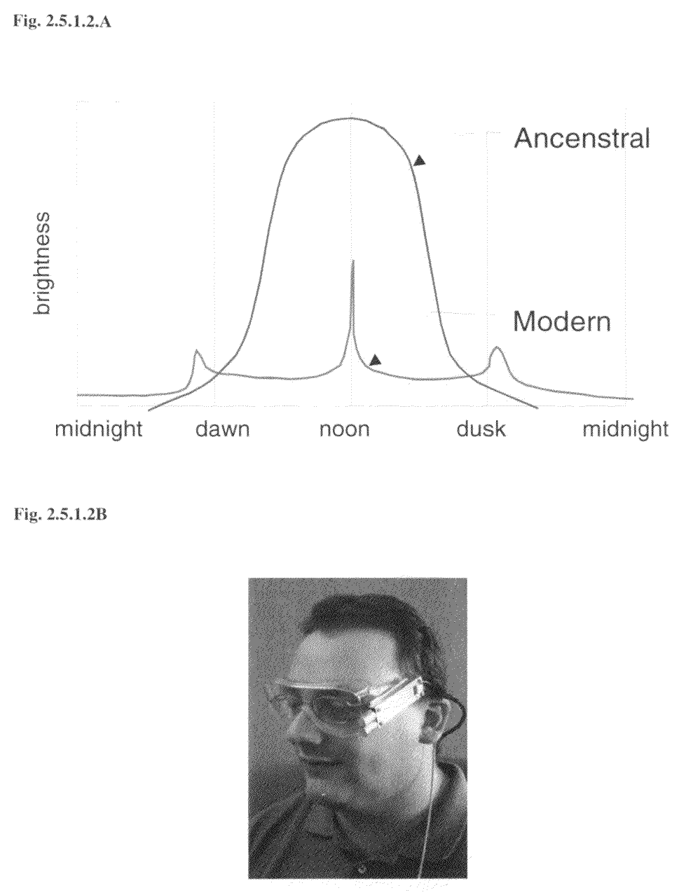

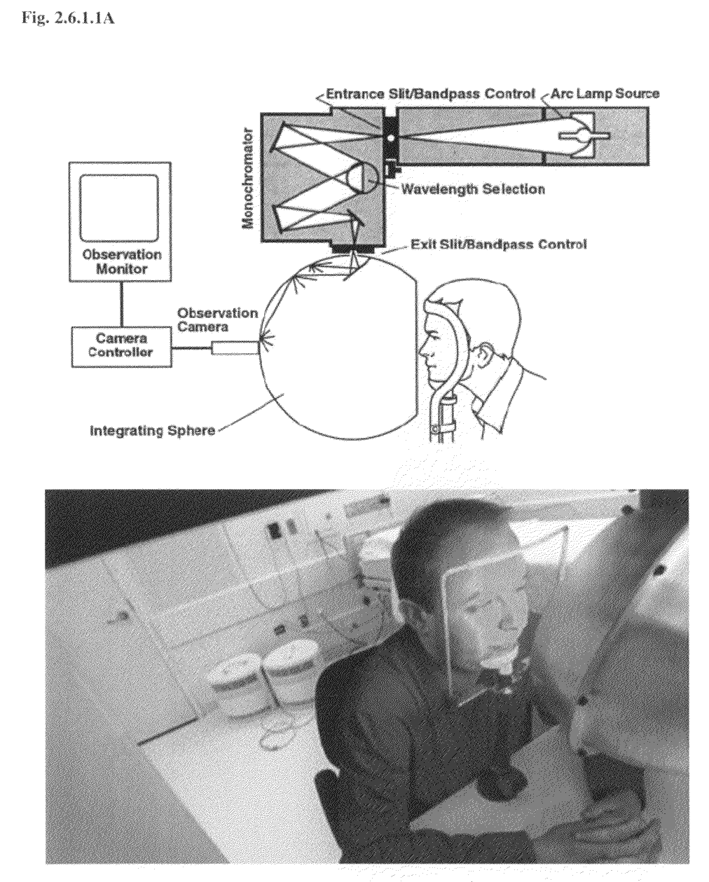

Many biological functions in the human body (and in all living beings in general) are rhythmic and follow a cyclic pattern. There are different cyclical processes operating at different rates depending on frequency. The circadian rhythm is driven by an internal clock or, in other words, an endogenous pacemaker that has a period close to, but rarely exactly, to the 24-hour day/night cycle. This rhythm regulates several activities such as the hormonal melatonin rhythm, core body temperature, sleeping and waking, level of alertness and other physiological parameters including cognitive function, performance, mood and immune responses. FIG. 2.1A shows four important circadian rhythms.

In any cycle, the difference in the level between peak and trough values is the amplitude of the rhythm. The timing of a reference point in the cycle (e.g., the peak) relative to a fixed event (e.g., as in circadian rhythms, the beginning of the night phase) is the phase. The time interval between phase reference points (e.g., two peaks) is called the period (see FIG. 2.1B).

The period of the circadian cycle is called tau. Tau is not synchronized, by default, with the natural 24-hour dark-cycle period. For the circadian cycle to function adaptively in the real world, the circadian system must be synchronized, or entrained with the astronomical day. If not entrained, the circadian cycle becomes a free running rhythm (FIG. 2.1C); biological rhythms will thus start at abnormal times during the day, and psychophysical disorders will follow. Therefore, this period (Tau) must be constrained to become exactly 24 hours (at least on average) in order for the biological rhythms to work optimally.

The endogenous pacemaker or the “internal clock” that drives the circadian cycle is located in the Suprachiasmatic Nucleus (SCN) of the hypothalamus (FIG. 2.1D). The circadian rhythms adopt a period that starts exactly every 24 hours when this internal clock (the pacemaker) is entrained with the day/night cycle.

DAILY ALTERNATION OF LIGHT AND DARKNESS: PRIMARY ENTRAINMENT FACTOR. It has been recognized in almost all of the experiments related to the circadian system that the dominant environmental entraining agent is the daily alternation of light and darkness. Prior to this discovery, scientists thought that social cues were the major factors that determined the daily human patterns (Lewy and Sack, 1996). Although other environmental cycles (i.e., temperature) can entrain circadian systems, it is the light-dark cycle that, because of its precision and reliability, has been used by natural selection as the major external temporal reference (Foster and Menaker, 1996). Otherwise, in constant dark or light conditions, the period of the free-run would be slightly off sync with the 24 hour cycle and consequently, the human activity period would shift a few minutes every day, disrupting many of the human psycho-physical activities.

In the dark/light cycle it is the light phase that plays an important role in regularizing the melatonin rhythm because it is light that controls the circadian production of melatonin (the importance of melatonin will later be explained). More specifically, it is visible light—the same light that we need for the visual system—that has an effect on melatonin's cycle and entrainment of the internal clock. Therefore, light is a crucial element for the internal clock's entrainment.

THE EYE: THE PERCEPTION ORGAN. The eye is the organ in which light information is transduced for the circadian system. Studies have shown that circadian rhythms cannot not be entrained by light cycles in the absence of the eyes (Foster and Menaker, 1996). Therefore, the eye is the receptor organ, not only for the visual system, but also for the circadian system. Importantly, the neuronal pathway for vision is anatomically separate from the pathway responsible for the circadian regulation (FIG. 2.3A).

Bright light entering the eye has an acute suppressive effect on the production of melatonin. When light does not reach the eye, the circadian melatonin cycle and consequently all psycho-physical cyclical behaviors enter into a free-running state. This happens, for example, in places where the sun is below the horizon for long periods of time (i.e., in Antarctica) and light does not reach the eye, or in the case of blind humans who lack the zone of the eye in which the circadian photoreceptors are located (Reiter, 1996). However, what is known is that light that reaches the retina has the greatest melatonin suppression effect. Although the exact location and chemical composition of the circadian photoreceptors in the eye are yet unknown, researchers believe that these photoreceptors are located on the retina of the eye (Foster and Menaker, 1996).

MELATONIN RHYTHM. Melatonin is a hormone whose main role is to reflect the environmental photoperiod via its secretion profile that follows a circadian pattern. Melatonin synthesis mainly occurs during the night (dark period) and less during the day (light period). Therefore, it can be considered as a humoral signal of darkness.

Melatonin is produced in the Pineal Gland and its production is regulated by the Suprachiastmatic Nucleus (SCN), the endogenous pacemaker. It has been supposed to influence sleep-awakening timing and thermoregulation in human beings, as well as reproductive development in some species (Kirsch, et. al., 1996).

When light enters the eye, the circadian photoreceptors transduce the information encoded in the characteristics of the light entering the eye. Receptors in the SCN receive this information and melatonin is produced within a cycle that is determined by this information. Therefore, melatonin is an important hormone for recording time and an important element for the regulation of the “internal clock.”

Encoded in the melatonin message is information about time of the day (clock) and time of year (calendar). Melatonin's production and amount of secretion depends on latitude and season. The ratio of light to darkness per 24 hour period varies seasonally and is more exaggerated at higher latitudes (see FIG. 2.4A). Since the duration of elevated melatonin at night is roughly proportional to the duration of the night, the melatonin signal thereby provides time-of-year-information (Reiter, 1996).

In this way, melatonin secretion is related to the length of the night: the longer the night, the longer the duration of the secretion. For example, the duration of melatonin secretion in extreme latitudes is much longer during winter nights and much shorter during summer nights than latitudes near the Equator. If a person lives in a region where night lasts 14 hours per day for a period of 2 months, the melatonin secretion will expand to cover almost the entire dark period and concomitantly, the circadian rhythm contracts to less than 10 hours (Arendt, 1996). This means that the circadian cycle will start its period earlier, causing a shift in the phase of the cycle; when the cycle is shifted, negative health consequences may occur.

HEALTH CONSEQUENCES IN THE PHASE SHIFTING OF THE CIRCADIAN RHYTHM. Depending on the time of the day that the light enters the eye, two main changes in the cycle are produced: a phase-delay (FIG. 2.5Aa) or phase-advance (FIG. 2.5Ab) in the circadian rhythm. The phase-advance/delays occur when the circadian biological rhythms are forced to be desynchronized with the environmental 24 hour light-dark cycle because light is provided at unusual times.

Phase-delay responses occur in the evening and decrease in magnitude during the middle of the night; phase advance responses occur in the morning and also increase in magnitude during the middle of the night (Lewy and Sack, 1996). A phase-advance implies that the psycho-physical rhythms start earlier (i.e., a person wakes up earlier) and a phase-delay implies that these rhythms will start later (i.e., a person falls asleep earlier). This is one reason why the timing of light exposure is so important; the clock mechanism is sensitive only at specified point of the cycle.

Psycho-physical disorders in human beings, such as seasonal depression in the winter, sleep disorders, and problems associated with jet lag and night shift work, are consequences of this de-synchronization with the environmental 24 hour light-dark cycle (Arendt, 1996). Therefore, in order to avoid health problems, it is important to reset the internal clock every 24 hours with proper light entering the eye.

FACTORS THAT LEAD TO MISMATCHES OF THE BIOLOGICAL CLOCK. Circadian rhythm variables are a mixture of endogenous and exogenous components: the endogenous component is controlled by the internal oscillator(s)—the body's internal clock (SCN)—and the exogenous component is controlled by aspects of environment and lifestyle. Certain aspects of the invention relate to the exogenous components which are related to the interior design of buildings.

In contemporary living conditions, humans are increasingly exposed to several factors that lead to a disparity between the internal clock and the 24 hour light-dark cycle through an altering of the circadian cycle. Light induction with “wrong” timing, an increased indoor life, and several aspects of social organization are some of the principal factors that lead to this disparity. An individual's lifestyle and environment provide misleading information when the phase of the endogenous oscillator(s) is inferred form overt circadian rhythms (Waterhouse, et. al., 1996).

LIGHT INDUCTION AT THE “WRONG” TIME. Today, people are more likely to experience light conditions that do not agree with the environmental light dark-cycle, especially those who live in urban areas. CIE recalls that Wehr published data suggesting that urban environments create biological darkness by day (in interiors with relatively low luminance) and unnatural brightness by night (electric lighting extending apparent day length); these conditions produce apparent constant day length over the seasons, with unknown health consequences (CIE, 2004).

In urban populations living under “normal” circumstances, it is likely that people perceive very small changes in day length during the year (Arendt, 1996). If natural light-dark cycle entrains the circadian rhythm of melatonin and therefore, the psycho-physical processes regulated by it, it is very likely to have light induction at unusual times, and therefore a melatonin suppression that will alter the internal clock's phase. For instance, light exposure after sunset for a long period (i.e., artificial indoor illumination in an office space) will give misleading information about the day's length, and therefore will cause a decrease in total melatonin production (Reiter, 1996).

INCREASED INDOOR LIFE AND LESS NATURAL LIGHT EXPOSURE. The primary cause of maladjustments in the biological clock has to do with contemporary lifestyle: people spend most of their time in interior spaces compared to the past, and therefore we obtain less exposure to natural light conditions and more exposure to artificial light conditions (see FIG. 2.5.1.2A). According to the U.S. Environmental Protection Agency, the average American spends 90 percent of his or her time indoors (EPA) where illumination conditions are far from adequate to correctly stimulate the circadian system. (As has been mentioned before, artificial light has been enhanced only to stimulate the visual system.)

When we spend the majority of our time indoors, we receive dim light for long periods of time rather than the bright light needed by the circadian system. In the industrialized world, total daily light exposure is low. Espiritu et al. found that the median person spends 4% of each 24 hour day exposed to illumination at levels greater than 1000 lux, and more than 50% of the time exposed to illumination levels from 1 to 100 lux (Espiritu et al., 1994; Koller et al., 1993) (see FIG. 2.5.1.2B and 2.5.1.2C).

Mark Rea and colleagues from the Lighting Research Center at RPI using a portable dosimeter (FIG. 2.5.1.2B) have measured the illuminance levels to which we are exposed in average indoor conditions. They have concluded that, in general, we receive between 1 lux to 500 lux; under outdoor lighting conditions, we would receive an average of 1000 lux on an overcast day and up to 10,000 lux on a clear day (FIG. 2.5.1.2C).

Another factor behind maladjustments to the biological clock is dim light itself, which not only fails to stimulate the circadian system but can also induce phase shifts if exposed to the eye for long periods. Indoor light, though 50 times lower in physical intensity than typical outdoor light, appears to have a significant accumulative effect: about 17 hours of 100-200 lux can shift the pacemaker phase by as much as 1 hour per day (Kronauer, Czeisler, et. al., 1996).

SOCIAL ORGANIZATION. In daily life, the patterning of the light stimulus is primarily dictated by the social constraints of employment and recreation which are tied to solar time. In the pre-industrial ages, artificial light was a scarce commodity and so the entrainment of the circadian pacemaker was governed by natural light, which is centered about noon (Kronauer, Czeisler, et. al., 1996). Current social organization leads to specific mismatches between our internal physiology and our environment.

Shift work and rapid travel across several time-zones leads to forced de-synchronization of internal rhythms from the external environment and from each other, with consequent problems of behavior (e.g., sleep), physiology (e.g., function) and performance (e.g., accidents). Similar pathological situations may result from imbalances in our biological clock, such as delayed sleep phase insomnia, some psychiatric disorders, and possibly some cancers and other pathologies (Arendt, 1996). These present-day entrainment phases appear to have two principal causes: reduced natural light exposure during daytime hours; and exposure to light at unusual times, including the extension of the illuminated hours well beyond sundown.

PSYCHO-PHYSICAL DISORDERS. The consequence of a de-synchronosis-divergence of the natural physiological rhythms of the organism between themselves, and with time, in other words, a desynchronized to the environmental lighting cycle, is a circumstance described as a free-running that leads to a psychophysical disorder (Klein, 1996).

Example psychophysical disorders in humans due to de-synchronization with the environmental 24 hour light-dark cycle include: (1) seasonal affective disorder (winter depression); (2) sleep disorders; (3) problems associated with jet lag and (4) shift work; (5) and cancer (Arendt, 1996). Certain aspects of the invention may be used to treat or prevent such psychophysical disorders.

Seasonal Affective Disorder (SAD) is a syndrome of recurrent depressive phases in autumn and in winter often associated with increased appetite, carbohydrate craving, weight gain, and hypersomnia. With SAD, these conditions improve spontaneously in spring and summer (Wirz-Justice, et. al., 1996).

SAD is more pronounced in geographical regions near the poles. In Florida, less than 1% of the general population experience SAD, while in Alaska as many as 10% may suffer from winter depression (Guzowsky, 2000). The so-called mid-winter insomnia (MI) occurs frequently in the far north as well as winter depression; this is a form of seasonal affective disorder (SAD) with a varying prevalence rate in relation to geographical latitude (Lacoste and Wetterberg, 1996).

SAD arises as a consequence of abnormally phase delayed circadian rhythms or of diminished circadian amplitude. Light is therefore an antidepressant because of its phase-advancing properties (when given in the morning) or its ability to enhance amplitude (when given in daytime) (Wetterberg, et. al., 1996).

Sleep disorders are related to circadian rhythm disruptions in which the individual experiences the following types of difficulties: inability to fall asleep until the early morning; falling asleep too early; sleeping episodically during the day; or suffering from constantly excessive sleepiness.

Delayed sleep phase insomnia (DSPI) is the most common intrinsic disorder of human sleep-wake rhythmicity with a reported prevalence rate of between 0.1 and 0.4% (Akerstedt and Folkard, 1996). DSPI can result in chronic sleep deprivation depending on the individual's need for sleep, and the need to rise early for scheduled activities. DSPI can be successfully treated with morning light particularly in combination with behavioral schedules (Arendt, 1996).

Shift workers tend to frequently change their sleep and waking times. Currently, it is estimated that approximately 20% of the workers in industrialized nations are shift workers (CIE, 2004). It should be emphasized that the circadian influence on sleep and wakefulness is dependant on the rate of adjustment of circadian rhythmicity to phase shifts of the sleep/wake pattern. With respect to shift work the adjustment is only marginal. Even in permanent night workers the circadian phase may only be delayed by 1 or 2 hours (Akerstedt and Folkard, 1996).

If sleepiness is excessive during work, one would expect an accompanying performance degradation and an increased accident risk at work (Akerstedt and Folkard, 1996). A number of studies have reported that shift workers feel particularly sleepy during the night shift. This type of behavior occurred in 25% of test subjects, and was not condoned by management or unions. The potential errors, accidents, and loss of productivity are significant when we realize that 20 percent of U.S. employees work night shifts (Guzowski, 2000).

Jet lag, understood as a condition resulting from rapid transport over several time zones, is a problem similar to that of shift work. Depending on the direction of travel (eastward or westward) and the number of time zones crossed (5 to 11), the typical human circadian system re-adjusts to such a challenge within three to twelve days (CIE, 2004). The adjustment to time zone shifts is approximately 1 hour per day after eastward flights and slightly more after westward flights. Interestingly, the effects of jet lag on human performance correspond to those seen in connection with blood alcohol levels of 0.05% (A kerstedt and Folkard, 1996).

The reasons that sleep disturbances result from shift work and jetlag seem rather straightforward. Controlled laboratory studies (under optimal sleep conditions) show that sleep that is displaced towards later times of night gradually decreases in length with increasing displacement. The short post-flight sleep of the tran-meridian jet traveler is a part of a general pattern of greatly reduced daytime sleep propensity, i.e. a time of day pattern (Akerstedt and Folkard, 1996).

In addition, evidence is mounting that melatonin suppression might increase cancer risk, and in particular, breast cancer (Hansen, 2001). The casual chain in this hypothesis is light exposure at night and the consequent suppression of melatonin. Among the indicators cited in the development of this hypothesis is the observation that breast cancer incidence is highest in developed countries where electric lighting is ubiquitous (Brainard, Kavet & Kheifets, 1999).

Data suggest that the lower the nocturnal peak of matonin the greater the probability that the tumor may be hormonally dependant. Thus, the modification of the melatonin rhythm may serve as a marker for increased risk of positive breast cancer development (Touitou and Haus, 1996).

RESETTING THE CLOCK. Remarkably, it is possible to re-synchronize the circadian clock by providing light to the eye. During the past decades researchers have proved that bright light is a stimulus for regulating circadian physiology and producing therapeutic benefits in patients with depression, sleep disorders, menstrual difficulties, as well as problems associated with jet lag and shift work.

In 1980, it was demonstrated that exposing the eyes of healthy humans to bright light at night causes a strong suppression of plasma melatonin (FIG. 2.6A). In that same year, Lewy and colleagues demonstrated that exposure to 2500 lux of white light during the night induced an 80% decrease in circulating melatonin within an hour. In contrast, volunteers exposed to 500 lux (less bright) of white light exhibited no significant melatonin suppression (Brainard, Gaddy, et. al., 1996).

Around that same year, experiments proved how bright light exposed at determined times advances or delays the phase of the clock rhythm. This finding opened the door to numerous studies on the biological and therapeutic effects of light in humans in order to re-set the human clock (Brainard, Gaddy, et. al., 1996).

After the discoveries of Lewy and other researchers about light and its suppressive effects on melatonin, numerous studies have been done to investigate how light can regulate human circadian rhythms—how can we “reset” the internal clock.

It is crucial to reset the human clock especially in people whose schedules are so important and in order to treat pathologically or socially induced disturbances of biological rhythms (Arendt, 1996). It has become clear that the human clock can be reset by suitable application of bright light. One disorder that has been commonly treated with light is seasonal depression (SAD). As Kjellman recalls, the first controlled study of light treatment in depression was performed by Kripke and coworkers in San Diego, where male patients with nonseasonal depression were treated with light. In 1982 Lewy and his coworkers reported the successful treatment with bright light in a patient with seasonal depression (Kjellman, et. al., 1993). Nowadays, the phase-shifting responses to bright light in the eye have been applied in the treatment of not only seasonal depression but also in advanced and delayed sleep phase syndromes, winter depression, jet lag and maladjustments from shift work.

Therapy for the reset of the clock basically consists in delivering bright light to the eye. Recent laboratory studies confirm that it is bright blue light delivered to the eye that has the maximum suppressive effect on melatonin. Spectral and intensity characteristics of the light necessary to strategically suppress melatonin in order to reset the clock have been studied from several studies and experiments conducted in laboratories. Georg Brainard, neurologist has developed many experiments through his career and he has recently conducted an experiment (2001) in order to establish the action spectra for melatonin suppression. The experiments defined an action spectrum that fits a retinaldehyde opsin template and identified 446-477 nm as the most potent wavelength region for regulating melatonin.

The following experiment description was taken from the article of “Action Spectrum for Melatonin Regulation in Humans: Evidence fro a Novel Circadian Photoreceptor” written by neurologist George Brainard and collegues (August, 2001).

The eye of 72 subjects (37 females and 35 males) was fully exposed to monochromatic lights. The subjects sat in a dimly lit room (10 luxes or less) in front of an apparatus that provided a uniform, patternless stimulus that encompassed the subject's entire visual field. The stimulus was emitted in an electronic, optic, dome exposure array (see FIG. 2.6.1.1A). The subject's head rested in an opthalmologic head holder facing an apparatus and was slightly withdrawn from the opening of the dome. During all light exposures, the subject's bony orbits are completely enclosed in the dome walls, providing completely exposure of their visual fields.

The light stimuli were produced by a 450 or 1200 W xenon arc lamp. An exit beam of light from each source was directed by a parabolic reflector. Each lamp was enclosed in alight-proof.

Monochromatic wavelengths (10-14.5 nm half-peak bandwidths) were produced by a grating monochromator, and light irradiance was controlled by a manual diaphragm. The resulting light beam was directed into the top area of a ganzfeld apparatus and reflected evenly off the walls of the ganzfeld dome into volunteer's eyes. The subjects gazed at a fix target dot in the center of the dome.

Experimental light stimuli reflected from the dome were measured at the person's eye level immediately before and after the 90 min exposure. Spectroradiometric assessment of the monochromatic wavelengths at the level of the person's corneas was done with a portable spectro-radiometer with a fiber optic sensor. Routine measurement of the light irradiance (in microwatts per square centimeter) was done with a Tetronix photometer.

The action spectra are determined by comparing the number of photons required for the same biological effect at different wavelengths. The melatonin suppression action spectrum described here was formed from fluence-response curves at eight wavelengths between 420 nm and 660 nm.

Characteristics of the material used to reflect the light towards the eye included: geometry (concave, dome shape structure encompassing each subject's entire visual system (parabolic reflector); surface property (patternless; reflective surface (how much is unknown); white coat material (Spectralite); and 95-99% reflectance efficiency over the 400-760 nm; characteristics of the light stimulus (spectrum: each wavelength was studied (420-600 nm), and intensity (31.8 uW)).

From the collected data, an action spectrum was determined by comparing the number of photons required for the same biological effect at different wavelengths. For this experiment, the action spectrum was formed by the photon density that had more effect in melatonin suppression for each of the 9 wavelengths; 464 nm was the wavelength predicted to be at the peak spectral sensitivity.

Now that specifically, controlled laboratory and clinical studies have demonstrated that light processed through the eye can influence human physiology, mood and behavior, there should be changes in future architectural lighting strategies. Complete specification of all aspects of the light stimulus is a requirement for research in this area. This must include a detailed description of the reflectances and surface characteristics of the setting as well as the spectral properties of the source, its intensity, its position relative to the viewer, and the optical properties of the luminaire.

### The Visual System Vs. the Circadian System

The visual and the circadian systems are affected by indoor light specifications, which therefore must be designed to enhance the functioning of both systems. As previously mentioned, the visual and circadian systems interpret light in different ways. As biological research uncovers the mechanisms of the circadian system, it becomes even more important for designers to specify indoor lighting and materials that work together to properly stimulate the circadian as well as visual system. One aspect of the invention relates to such lighting and materials. Interior light systems have been greatly enhanced to provide visual clarity, herein it is proposed that new lighting specifications must be developed for the circadian system.

To analyze the quality of light reaching the eye, one must begin with a description of the spectral properties of the light stimulus. The surface characteristics of the space surrounding the subject must also be taken into consideration because it is the interaction of the source light with a room's surface properties that determines the color, amount, and distribution of light. The interaction between light and material properties determines the resulting light characteristics that reach the eye. The spectrum and intensity of the light source, the duration of the light stimulus, the time of the day at which the light stimulus is delivered, and the spatial distribution of the overall lighting system are characteristics that must be considered both for the visual and the circadian systems.

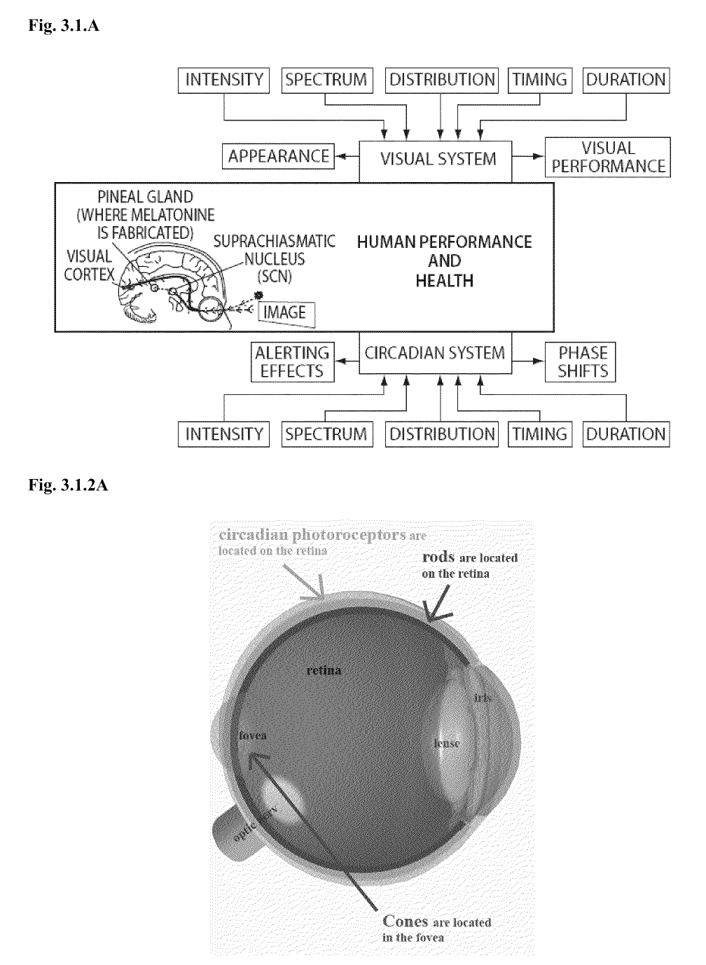

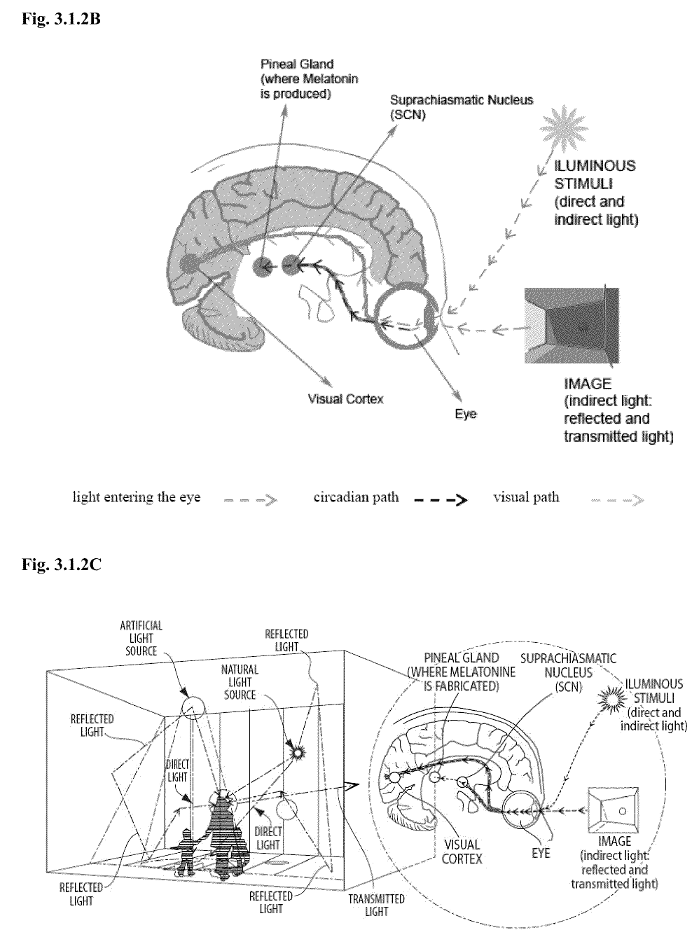

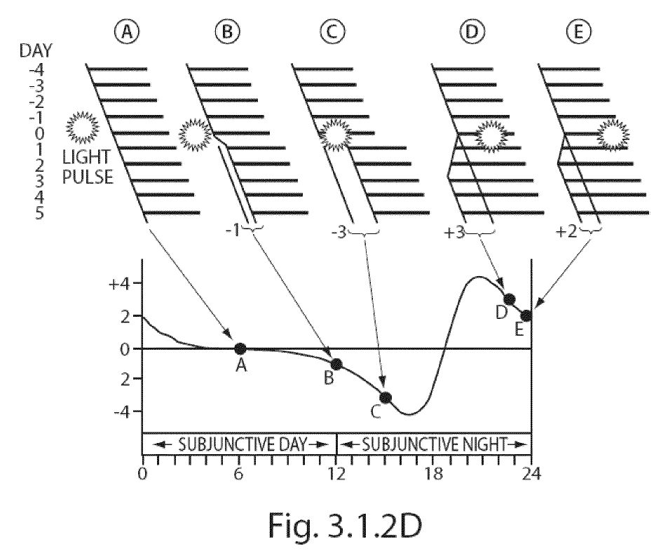

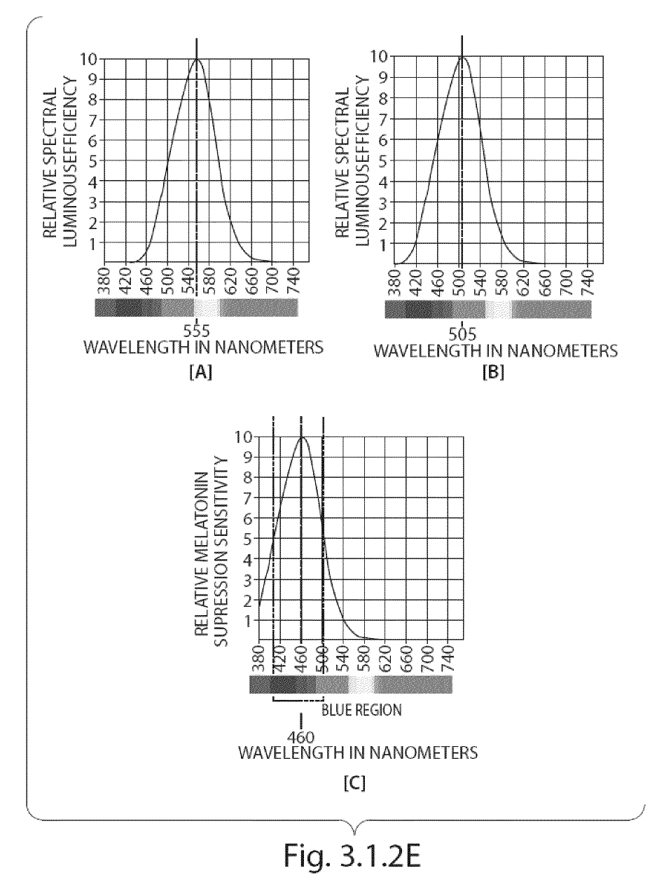

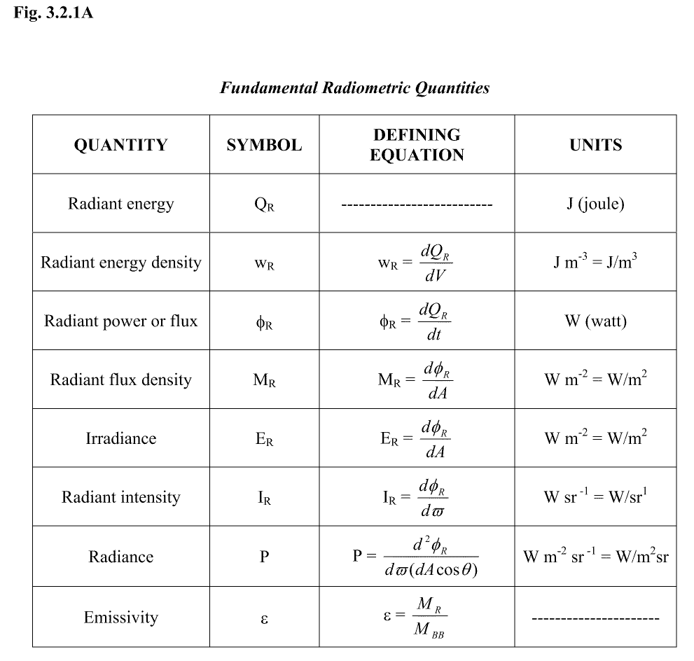

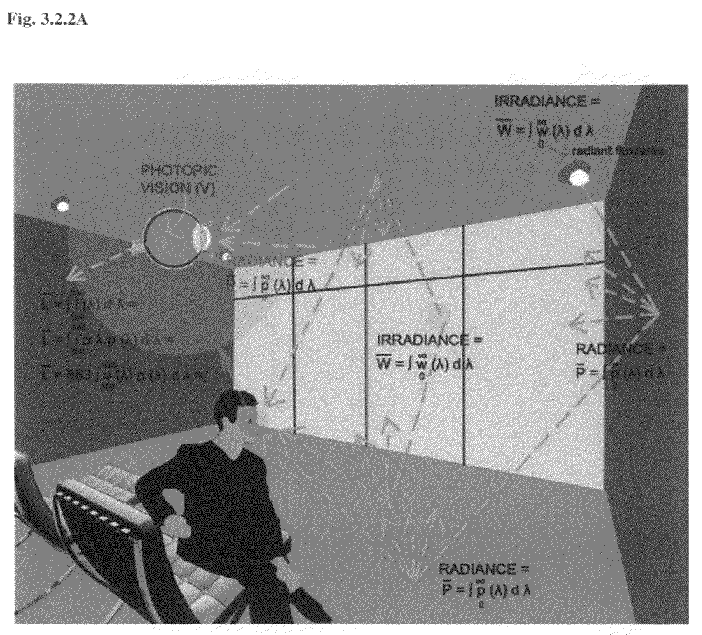

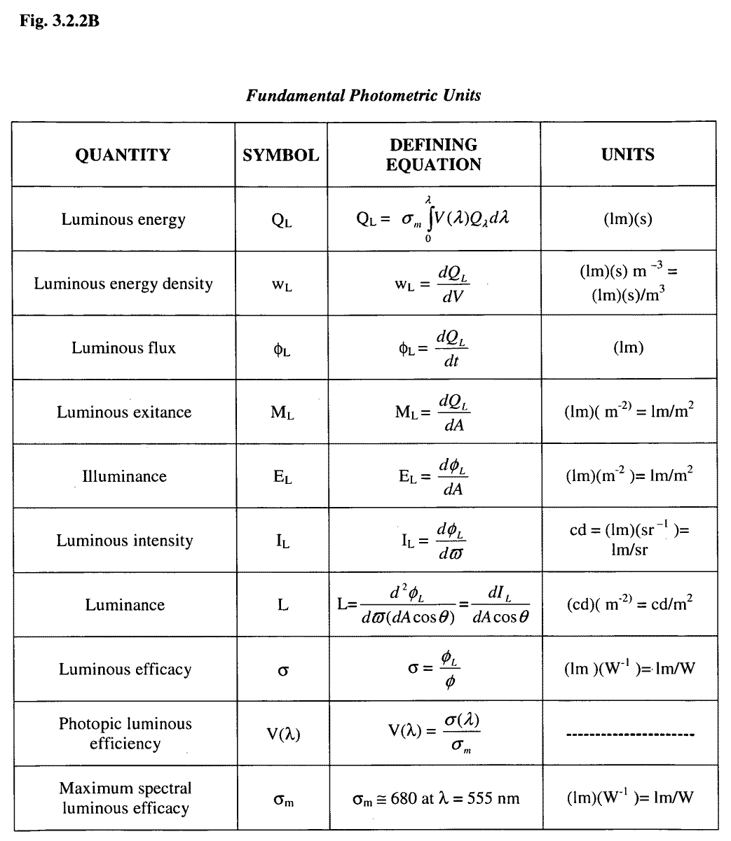

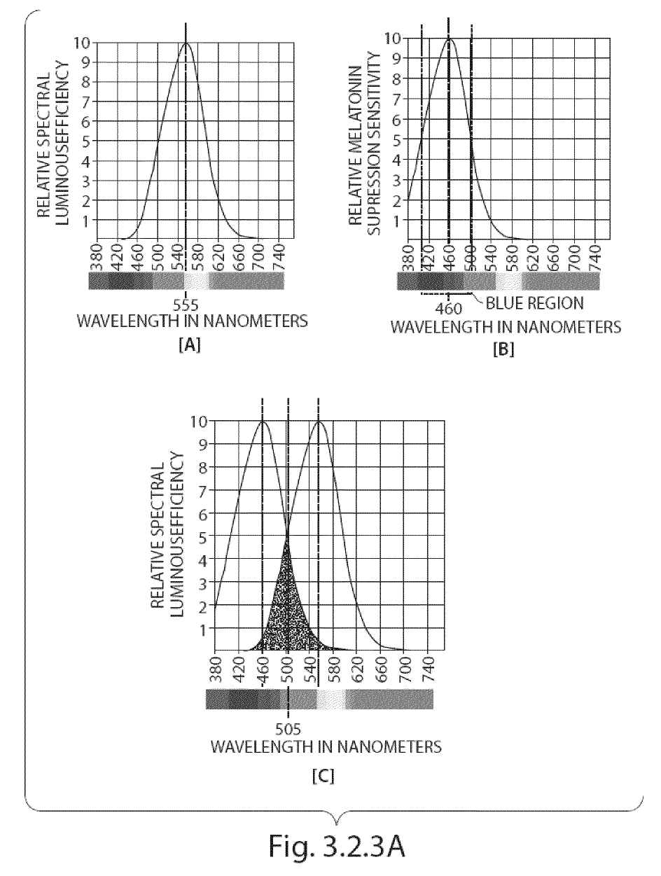

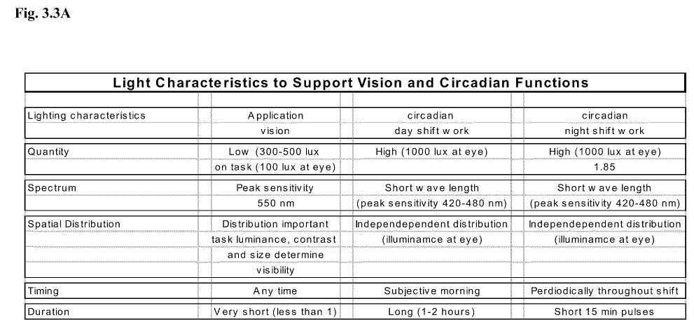

SIMILARITIES AND DIFFERENCES OF THE VISUAL AND CIRCADIAN SYSTEMS. The visual system and the circadian system have similarities and differences in how they work and how they contribute to human cognitive performance. The biggest similarity is that both systems share (1) the eye as the organ transferring data to be interpreted by the brain and (2) light energy as the information source. However, both systems interpret light in different ways for different purposes (see FIG. 3.1A).

As described above, the circadian system interprets the information from visible light in order to regulate biological functions such as sleep and wakefulness, body temperature, hormonal secretion, and other physiological parameters including cognitive function and immune responses. The circadian system is also related to psycho-physical functions such as mood, performance, and alertness. The visual system interprets the information from visible light to build a representation of the world surrounding the body. It reconstructs a three dimensional world from a two dimensional projection of that world.

Both systems contribute to the cognitive performance of the human body; both systems use visible light as the primary stimulus and information source; the organ for light transduction is the eye—the perception organ; and both systems detect spectrum and intensity as the primary light characteristics transduced in electrical energy for the brain.

However, the photoreceptors in both systems are different (see FIG. 3.1.2A). In the bisual system the two light receptors are rods and cones, which have different properties. These receptors contain light-sensitive chemicals called visual pigments that react to light and trigger electrical signals. In the circadian system the photoreceptors have not been fully determined, but research by several neurologists, such as Brainard, suggests that the light circadian receptor must be a photopigment based on retinaldehyde located on the retina of the eye (Brainard, Hanigin, et. al., 2001).

In addition, the neuronal path is different for both systems (see FIG. 3.1.2B). In the visual system: light energy is transduced by the eye's visual photoreceptors and sent to the visual cortex through the optical nerve. In the circadian system: light energy is transduced by the circadian photoreceptors and sent to the suprachiasmatic nucleus through the circadian neural path.

Further, the light source does not have be delivered to be eye in the same way (see FIG. 3.1.2C). For the visual system to detect an image, the eye has to perceive luminance, which is the luminous intensity projected area of a surface in a given direction (light reflected from the surface of an object) (Wibom, 1993). In other words, the visual system only records light that has interacted with the material of the surface of an object (indirect light). However, the circadian system detects both direct and indirect light. It detects light that has already interacted with a material (luminance) or light that is directly radiated by a light source without interacting with any material (radiance).

Yet another difference is that the time of light exposure to the eye for the systems to be stimulated is different. We can see at any time (night or day) as long as there is enough intensity for the visual system to be stimulated (Rea, 2002). However, the time of the day when light has to be exposed to the eye in order to suppress melatonin secretion is in the morning or evening, respectively, if the desired result is a phase advance or a phase delay of the circadian rhythm (Rea, 2002) (see FIG. 3.1.2D).

Another difference is that the peak of spectral sensitivity is different for both systems. The peak of spectral sensitivity for the visual system is at 555 nm during the day (for photopic vision) (see FIG. 3.1.2Ea) and at 505 nm during the night (scotopic vision) (see FIG. 3.1.2Eb) However, the peak of spectral sensitivity for the circadian system resides at the range of 446 to 477 nm (Brainard, Hanigin, et. al., 2001) (see FIG. 3.1.2Ec).

In addition, the peak of intensity sensitivity is different for both systems: for the visual system it varies from 100-500 lux (Rea, 2002); and for the circadian system is higher than that necessary for the visual system: from 1000 lux (Rea, 2002).

Further, the duration of light exposure in order for the systems to be stimulated is different. The duration of light exposure needed in order to see is very small (less than a second). We can perceive the characteristics of the surrounding environment at the moment when light becomes available. The duration of light exposure needed in order to stimulate the regulation of melatonin secretion varies, but is approximately 1-2 hours (Rea, 2002).

Finally, the spatial distribution of light entering the eye is different as well. We see objects because light is reflected into our eyes and therefore, for the visual system the spatial distribution of light is very important. The visual system depends on the preservation of ray geometry. For the circadian system the means by which the light energy is delivered to the eye is more important than the overall ray geometry of the light in a specific space (Rea, 2002).

UNITS FOR THE MEASUREMENT OF THE LIGHT STIMULUS. Because the photoreceptors in both systems are different, the light stimulus reaching the eye has to be measured in different units. Light for the visual system has been measured in what are called photometric units. Because the photoreceptors for the circadian system have not yet been completely studied, the units to measure circadian light have not yet been determined. Therefore, the units that have generally been used to study the circadian system are radiometric.

Usually, physicists and engineers use radiometric units to measure light, while in lighting design and architecture photometric units are preferred. The first are based on the physics and behavior of the light without taking into account the visual photoreceptors; photometric units, on the other hand, are based on the perception of light through the visual photoreceptors.

In telecommunications and physics, radiometry is the field that studies the measurement of electromagnetic radiation, including visible light. It is the science of measurement of light in terms of absolute power. See FIG. 3.2.1B.

Light is radiant energy. Energy is measured in joules. Light propagates through media such as space, air, and water. Light can be measured over time, space, or angle. This radiant energy is indicated as radiant power or radiant flux in joules per second, which is equal to watts. Radiant flux density is the radiant flux per unit area, known as irradiance when this flux is arriving from all possible directions. Irradiance is measured in watts per square meters. Therefore, these are measurements of energy per unit of time as well as per unit of area.

If we consider an infinitesimally small point light source, the light emitted in a particular direction is called radiant intensity measured in watts per steradian. A steradian is a measure of a solid angle corresponding to an area on unit sphere. Radiant intensity thus measures energy per unit of time per unit of direction.

Flux passing through, leaving, or arriving at a point in a particular direction is known as radiance and is measured in watts per square meter per steradian. It is a measure of energy per unit of time, per unit of area, as well as per unit of direction.

Photometry is the science of measuring light in terms of its perceived brightness to the human eye. In other words, it is the measurement of light defined as electromagnetic radiation detectable by the human eye.

The human eye is not equally sensitive to all wavelengths of light. Photometry attempts to account for this by weighing the measured power at each wavelength with a factor that represents how sensitive the eye is at that wavelength. The standardized model of the eye's response to light as a function of wavelength is given by the luminosity function.

Because we are typically interested in how humans perceive light, light's spectral composition may be weighted according to V(λ). The science of measuring light in units that are weighted in this fashion is called photometry. All radiometric terms introduced in the previous section (radiometry) have photometric counterparts (FIG. 3.2.2B). By spectrally weighing radiometric quantities with V(λ), they can be converted into photometric quantities.

Luminous flux (or luminous power) is photo-metrically weighted radiant flux. It is measured in lumens, which is defined as 1/683 watts of radiant power at a frequency of 540×1012 Hz. This frequency corresponds to the wavelength at which humans are sensitive (about 555 nm).

If luminous flux is measured over a different angle, the quantity obtained is luminous intensity, measured in lumens per steradian. One lumen per steradian is equivalent to one candela. Luminous exitance and illuminance are both given in lumens per square meter, whereas luminance is specified in candela per square meter (nits).

Luminance is a perceived quantity. It is a photo-metrically weighted radiance and constitutes an approximate measure of how bright a surface appears.

In this way, each of the quantities given in the table of radiometry may also be defined per unit wavelength interval, which is then referred to as spectral radiance L, spectral flux P, and so on. The subscript e indicates radiometric quantities and differentiates them from photometric quantities. FIG. 3.2.2A describes how light is irradiated form the main light sources (natural and artificial light, how it radiates form the surfaces in a room and then how the quantity of light is interpreted by the eye (photopic light).

PHOTOPIC AND CIRCADIAN SENSITIVITY CURVE. Even though the human eye is not equally sensitive to all wavelengths of the visible spectrum, the range of sensitivity is small enough that the spectral sensitivity of any human observer with normal vision may be approximated with a single curve. Such a curve is standardized by the Comission Internationale de l'Eclairage (CIE) and is known as the V(λ) curve (pronounced as vee lambda), or the CIE photopic luminous efficiency curve (see FIG. 3.2.3Aa).

Similarly to the photopic sensitive curve, a circadian sensitivity curve has recently been calculated (FIG. 3.2.3Ab). George Brainard and colleagues performed a study to establish an action spectrum for light induced melatonin suppression. Interestingly, the curve shows a Gaussian form very similar to the photopic sensitivity curve. The main difference is that its peak ranges from 446 to 477 nm (Brainard, Hanigin, et. al., 2001) (see FIG. 3.2.3Ac).

PROBLEM OF MEASUREMENT. However, photo-transduction of light by the circadian system seems to be performed without registration, and the retina serves as a simple integrator of photon absorption. Many scientists and clinicians working on the circadian, neuro-endocrine, and therapeutic effects of light in humans have predominantly used photopic illuminance as their standard light measurement. However, if the normal, three cone visual system is not the photoreceptor system through which light information is transduced for other physiological systems (circadian system), then the use of photopic photometric measures for non-visual effects of light becomes questionable (CIE, 2004).

Further, CIE states that the spectral response data reported since 2001 suggests that photopic photometry is a poor measurement system for some of the non-visual effects of light. For example, for light sources commonly used in offices, schools, and homes, errors in spectral characterization of light for the circadian system can be as much as 3:1 (Rea, 2000). These findings have serious implications for accurate measurement.

CIE and some light researchers such as Mark Rea have even started mentioning that a new system of measurement for the circadian system must be proposed in order to support human health; without the formality of such a system, it would be much harder to develop the best lighting technologies and applications for human health (Rea, 2000). We must start to think of a different measurement of light because the spectrum, intensity, duration, timing, and spatial distribution for circadian impression are radically different than those required for vision. Meanwhile, for purposes of studying the circadian system, most of the neurologists are using radiometric units.

FIVE IMPORTANT LIGHT PARAMETERS. It is becoming clear that we have consider new ways of measuring light and indoor light systems to enhance the circadian system as well as the visual system. Five principle light characteristics differ between the circadian and the visual system. These five parameters in light are spectrum, intensity, duration, timing, and spatial distribution. See, for example, “Much More than Vision,” Mark S. Rea, 2002.

One important parameter is spectrum. There is a marked disparity between the spectral response photometers (for the photopic vision) and the spectral sensitivity of the human circadian vision. However, short-wavelength light sources stimulate the human circadian system much more effectively than long-wavelength light sources. It has been proven that the spectrum peak of sensitivity for the circadian system is around 460 nm as opposed to 555 nm for the photopic vision. Light sources rich in short wavelengths (i.e., daylight) will be seriously under-represented by conventional photometric measurements.

Another important parameter is intensity. Many studies have confirmed that the intensity needed to stimulate the human circadian system is much more than the intensity needed for the visual system. Illuminance levels at an average office space are usually adequate for the visual system to perform at its maximum. However, this intensity does not stimulate the human circadian system. Bright light is necessary for a good melatonin regulation. It is true that dim light exposed during long times can suppress melatonin and shift the circadian rhythm. However, this light is not optimal for circadian synchronization and may have an accumulative effect leading to the proportion of wrong information about the natural light-dark cycle.

Another important parameter is duration. As opposed to the visual system, the circadian system does not respond as quickly to light exposure: the circadian system responds to a light stimulus with a lapse of 1 to 2 hours. The difference is that the visual system works within a rapid neural circuit and the circadian system relies on the infusion of the hormone melatonin into the blood stream to communicate to various systems in the body.

Another important parameter is timing. The time of the day is non-important for the visual system to work in an optimal way. We can see at any time of the day if the light levels are adequate. However, for the circadian system this is not true. The temporal characteristics of light are important to the circadian system and must be considered in any system of circadian photometry. Depending upon light exposure, there can be a phase delay, a phase advance, or no effect in phase shift at all. If light is applied in the first half of the night, the clock is reset to a later night (phase delayed), whereas this same light applied in the second half of the night will reset the clock to an earlier time (phase advanced).

Another important parameter is spatial distribution. Accurate registration of the spatial distribution is important for the visual system. In order to get a good definition of the environment that surrounds us, we have to have a clear distinction between dark and bright patches of light that together give us a clear idea of the 3D space we inhabit. However, accurate registration of spatial information on the retina is not important for the stimulation of the circadian rhythm. Photo-transduction of light by the circadian system seems to be performed without registration, and the retina serves as a simple integrator of photon absorption.

On the other hand, the direction of the light is important for the circadian system. As mentioned before, in order to be effective, light has to enter the eye and, more specifically, it has to reach the retina (Brainard, et. al., 1996).

FIG. 3.3A summarizes the five light characteristics in relation to the circadian and the visual systems.

One aspect of the invention relates to controlling the first three light characteristics listed above: quantity, spectrum, and spatial distribution. In an interior environment, the precise nature of these characteristics results from the characteristics of the interior surfaces and the spectral properties of the source light, including its position relative to the viewer and the optical properties of its luminaire. In other words, quantity, spectrum, and spatial distribution can be understood as resulting from specifically architectural parameters—interior form, surface, material, orientation, etc.

### Interaction Between Light and Material Properties

Light emitted from a primary light source (i.e., the sun or a lamp) does not generally enter the eye directly; rather, it usually arrives after having interacted with the material surfaces of surrounding objects. Although a light beam can, for example, arrive to the eye directly from the bulb of a lamp, it is more likely in interior conditions that the light reflects off of surrounding surfaces and objects before entering the eye.

The spectral composition and intensity of light varies depending on how the light interacts with given materials, and depending on the optical properties of the surface material of objects. Therefore, the light stimulus penetrating the eye conveys spectral information of both the light source illuminating a surface point and the optical properties of the surface at that point; this stimulus resulting from the light source and material properties is captured by the circadian photoreceptors and transduced by them.

When an object is illuminated, light interacts with the surface of the material in different ways depending on the material's optical or atomic configuration. If the material is transparent, light can be transmitted, reflected, or refracted. If the material is translucent, light can be transmitted through it, reflected, refracted, partially scattered, or partially absorbed. If the material is opaque, light can be reflected, absorbed, scattered, or re-emitted.

These five transmissions—reflection, refraction, scattering, emission, and absorption—can change the color and intensity of the original light source interacting with the material since each wavelength generated from the light source reacts with the material differently. Several of the above transmission phenomena can occur simultaneously, depending on the material characteristics.

The description of these phenomena below is based on the books titled Optics by Eugene Hecht and Alfred Zajac (Hecht, 1974), and Colour and Optical Properties of Materials by Richard Tilley (Tilley, 2000).

LIGHT. Throughout the history of studies in Physics, researchers have struggled to understand the behavior of luminous phenomena. Today, we refer to light as an electromagnetic wave when its interaction with matter is studied from (1) the macroscopic point of view or (2) the atomic level or microscopic point of view, where light is understood as a particle—a photon. Given these two conceptions of light, we are dealing with a phenomenon that presents a wave-particle duality. The majority of the illumination phenomena described herein can be studied from the macroscopic point of view whereby light is treated as a wave. However, in some cases, and whenever pertinent, light will be considered as a photon beam.

Herein, light will be considered as a wave and a photon in order to explain the main processes for its interactions with matter. Together, these processes explain the conditions by which the human eye perceives the surrounding environment.

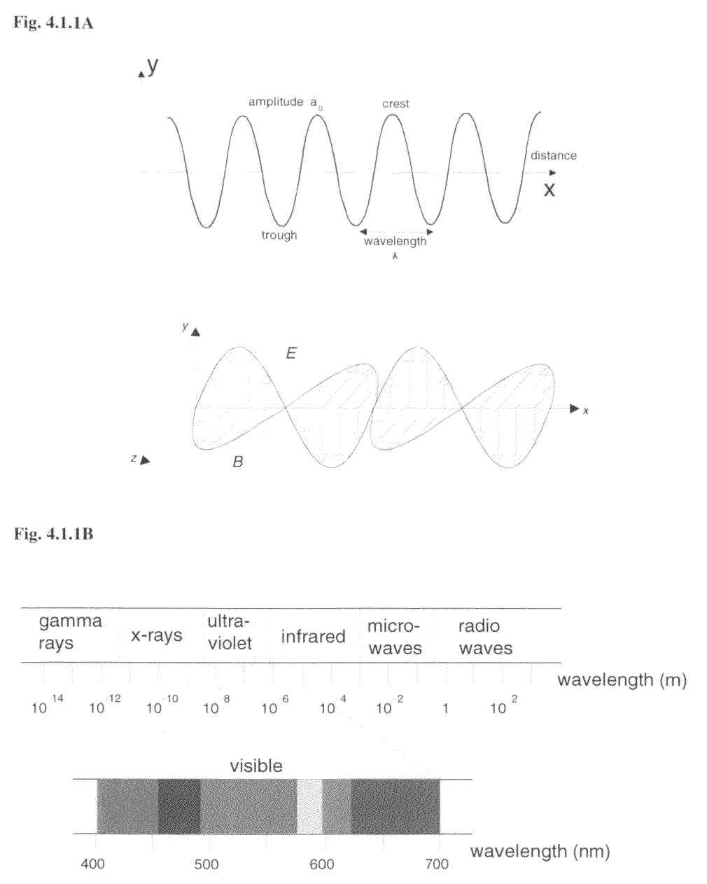

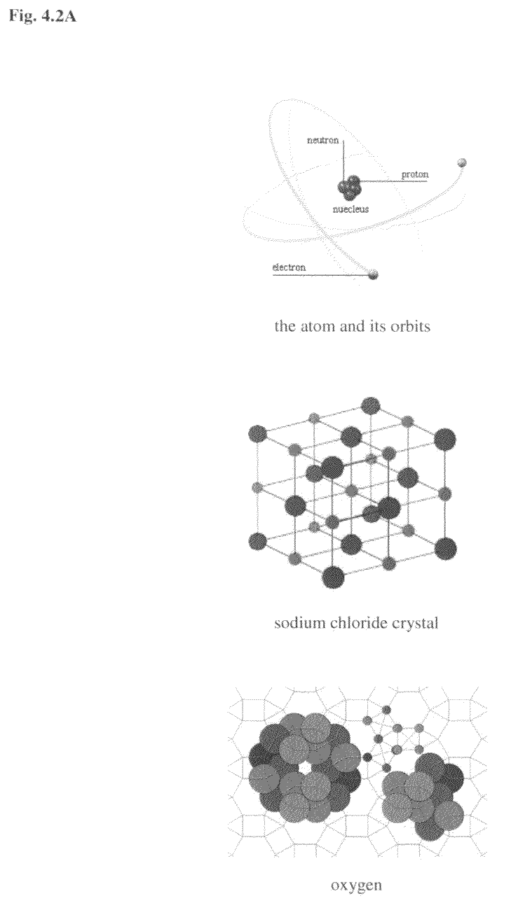

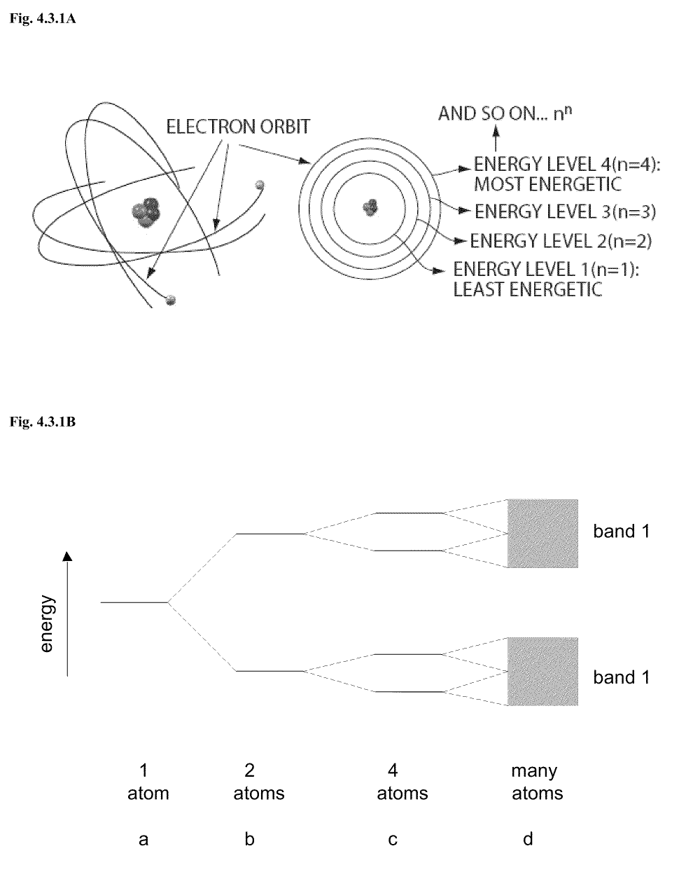

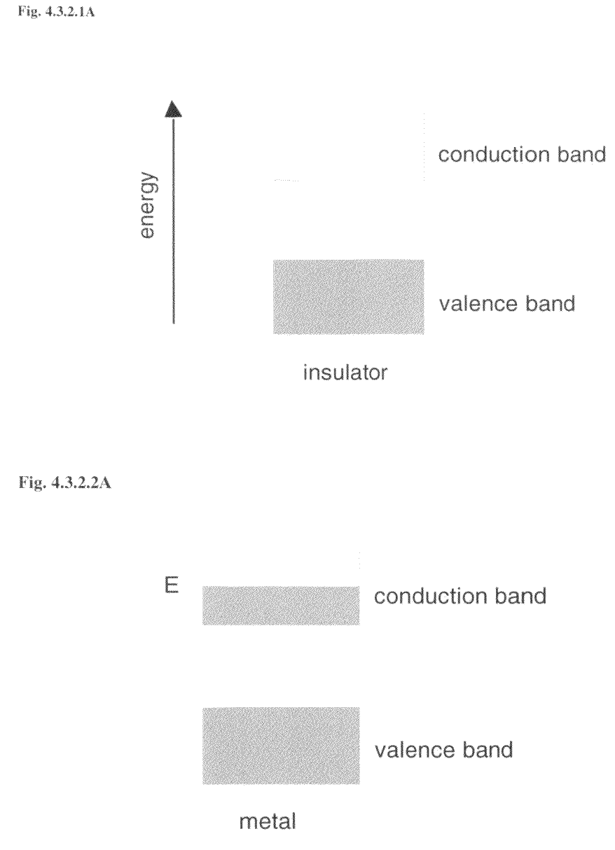

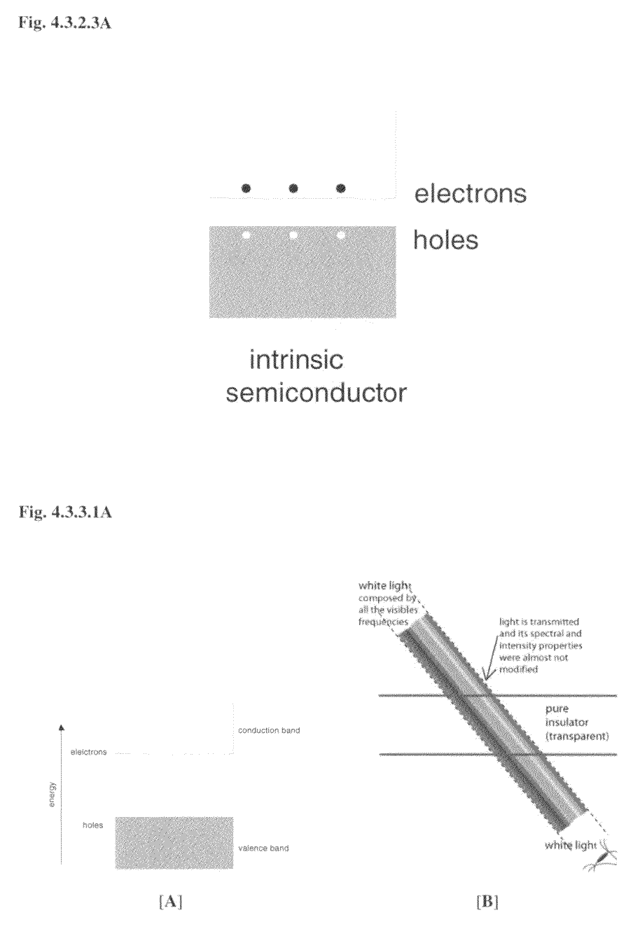

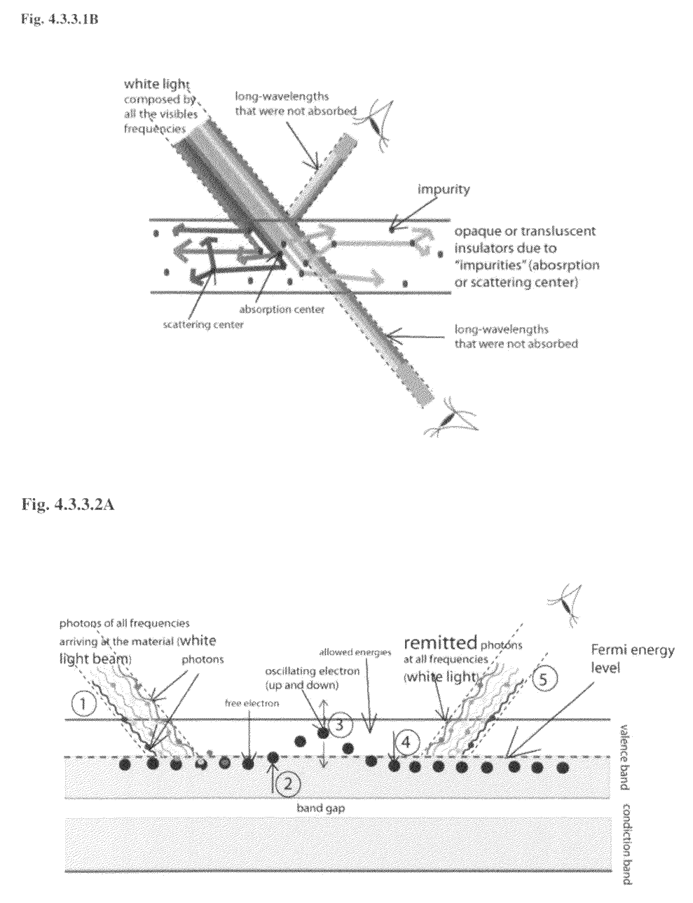

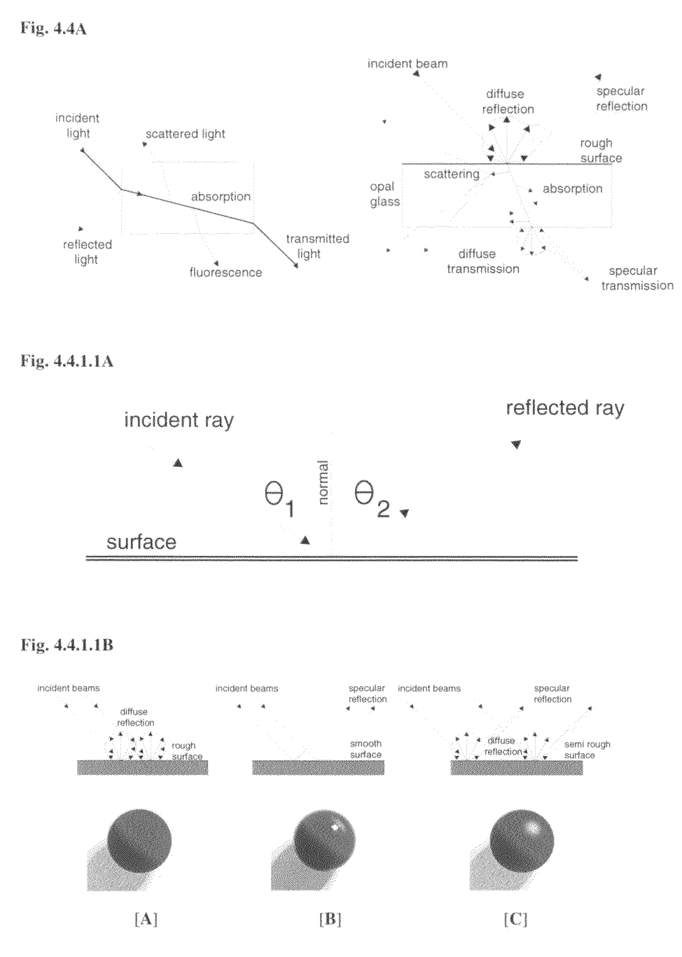

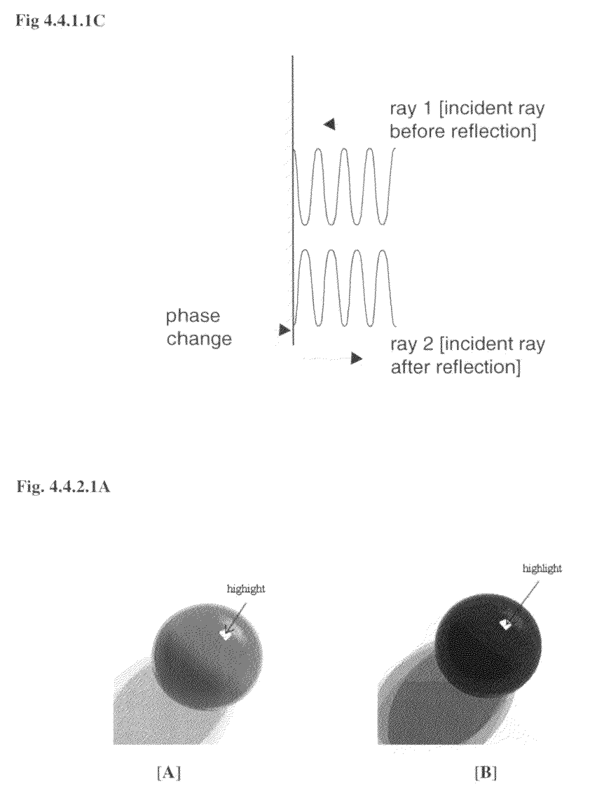

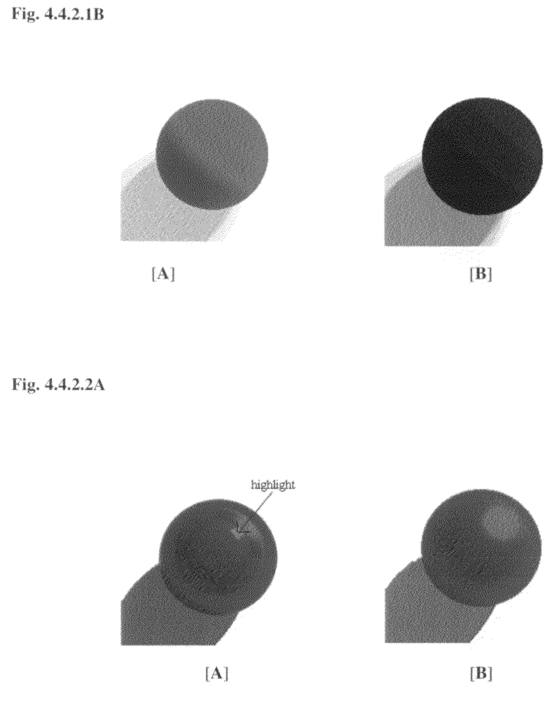

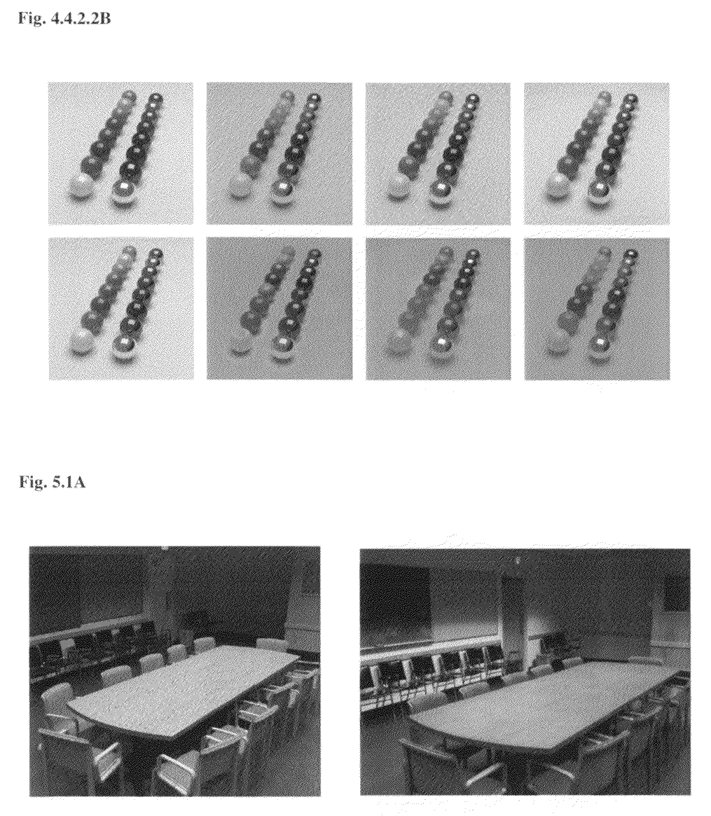

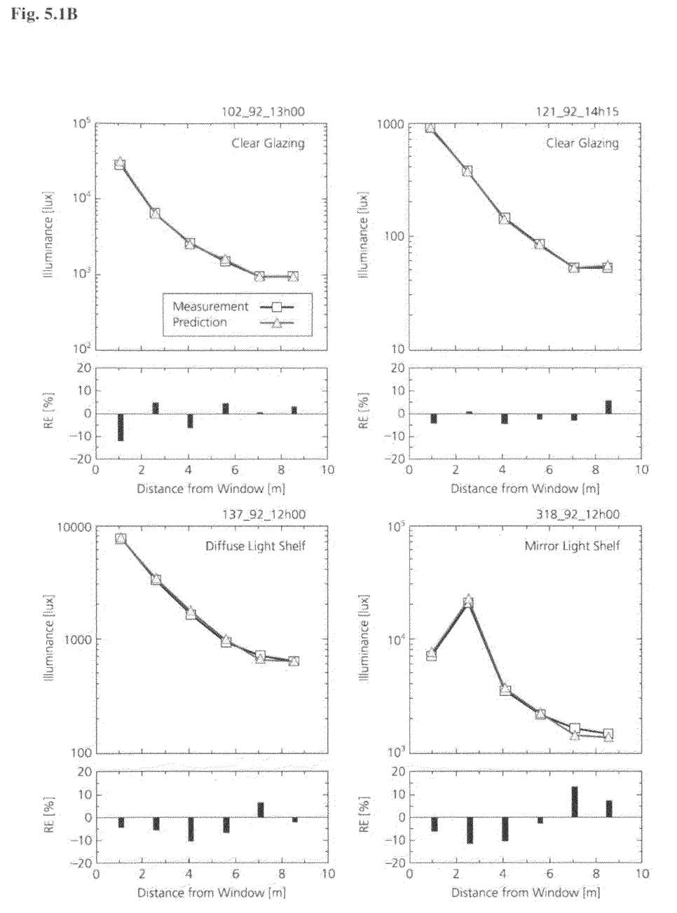

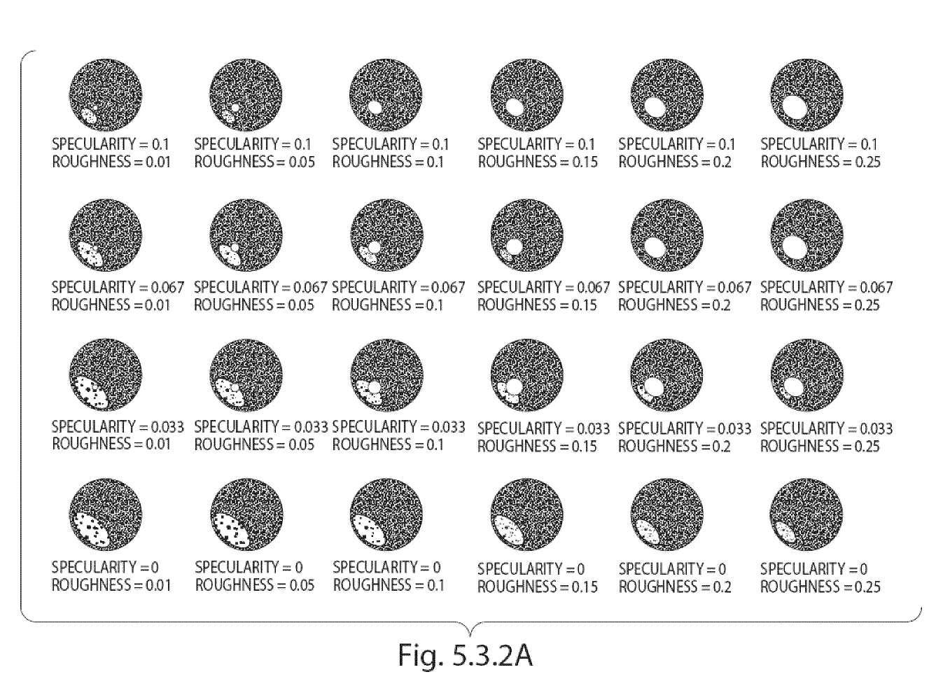

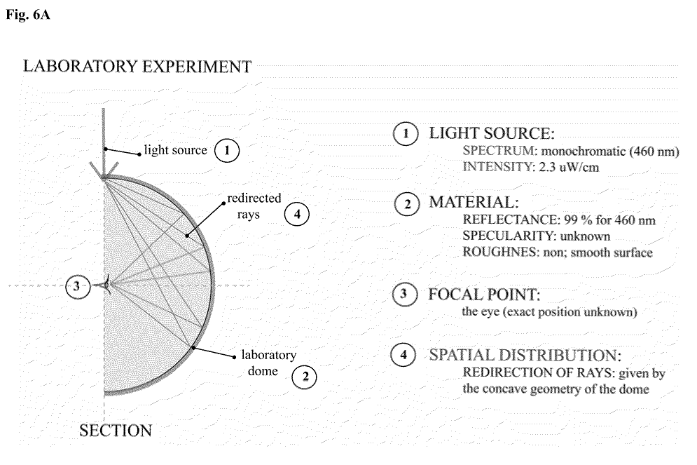

Light is described as a wave composed by two vectors, a magnetic field vector B and an electric field vector E, that travel in space perpendicular to each other and oscillating like a sinus function. These two vectors travel in a plane perpendicular to light's propagation direction as shown in FIGS. 4.1.1Aa and 4.1.1Ab.

The electromagnetic spectrum is very broad; the amplitude of its waves range from 10-14 to 102 m. Of this entire spectrum, the visible region, commonly referred as visible light, ranges from 400 to the 700 nm (within the region of the 10-9 m) (see FIG. 4.1.1B)

Light in the visible spectrum has wavelengths that are associated with different colors and levels of energy. Wavelengths from about 400 to 450 nm appear violet; 450 to 490 nm appear blue; 500 to 575 to 590 nm appear yellow; 590 to 620 nm appear orange; and 620 to 700 mm appear red (Bruce Goldstein, page 187).

The two extreme ends of the spectrum—visible red and visible violet—have rather different personalities. Red light is related with heat (i.e., an object begins to radiate light as it is heated).

Therefore, the blue-violet end of the spectrum reaches a high intensity only when the radiating body is exceedingly hot (the blue end of the spectrum is the more energetic end). Indeed there exists a specific energy per amount of light that is much higher in blue-violet than in red light.

MATTER. Matter is commonly defined as the substance of which physical objects are composed. It is composed by atoms (electrons, protons, and neutrons) that arrange in different configurations. It is these arrangements in conjunction with the behavior of the atoms that give specific properties to different materials (see FIG. 4.2A).

When an electromagnetic wave-photon interacts with the surface of a material, the electromagnetic field affects the electrons of that material. Consequently, different material structures give off different colors and intensities in the resultant light.

Materials are formed by various kinds and arrangements of atoms and molecules that give different materials particular spectral intaraction characteristics: reflection, refraction, scattering, absorption, or emittance. For example, each opaque or transparent material has its own characteristic color when illuminated with the same white light.

Solid materials, according their electronic configuration, are classified as dielectrics (insulators), metals, and semiconductors. Each type of solid interacts differently with electromagnetic radiation at a microscopic scale (photon-electron), and this difference determines a solid's optical properties.

In order to understand the optical properties of solids, and how light photons interact with dielectrics, metals, and semiconductors, one first has to explain their atomic configurations.

ELECTRONIC CONFIGURATION OF SOLIDS. Electrons in an atom have well defined energies and revolve in suitable orbits around the atomic nucleus. The energies for these orbits are called electronic energy levels (see 4.3.1A). The microscopic light interaction with solids can be understood in this context, and one of the best models for this purpose is the band theory. In the band theory approach, electron energy levels are conceived as broadened energy bands. In an isolated atom the electrons occupy a ladder of sharp energy levels. In FIG. 4.3.1Ba, the outermost energy level of an isolated atom is shown. If another atom approaches the first, then the outer electron clouds will interact and the single energy level will split into two—one at a higher energy and one at a lower energy, as shown in FIG. 4.3.1Ab. Four atoms will give four energy levels, as illustrated in FIG. 4.3.1Ac.

This process can be continued indefinitely. As each atom is added to the cluster, the number of energy levels in the high energy and low energy groups increases. Ultimately, when a large number of atoms are brought together, as in a solid, the energy levels in both the high energy and low energy groups are very close indeed. They are now called energy bands and are shown in FIG. 4.3.1Ad.

The details of the band structure of a material depend upon both the geometry of the structure and the degree of interaction of the electron energy levels. If the interaction is large, typically for the outer orbital of closely spaced large atoms, the bands are broad. When the interaction is lower, as occurs for inner electron orbitals on atoms which are further apart, the width of each band is rather narrow. The electrons in the solid fill the bands from the lowest energy to the highest. The topmost band can be partly (metals) or completely full (dielectrics and semiconductors) as discussed below in more detail.

PHOTON AND ELECTRON INTERACTION. Dielectrics are usually transparent or translucent material. Light can be transmitted, reflected, refracted, partially absorbed, and scattered. If the absorption is very strong, then, these materials appear opaque. Dielectrics might also be called insulators because they are poor conductors of electricity due to their atomic configuration. Metals are always opaque: light is mostly reflected and absorbed, and they are very good conductors of electricity. Semiconductors can be explained as materials whose configuration is in between metals and dielectrics.

In insulator materials, the upper energy band is completely empty and the lower energy band is completely filled by electrons, as illustrated in FIG. 4.3.2.1A. Moreover, the energy gap between the top of the filled band and the bottom of the empty band is quite large. The filled energy band is called the valence band and the empty energy band is called the conduction band. The energy difference between the top of the valence band and the bottom of the conduction band is called the band gap.

Metals are defined as materials in which the uppermost energy band is only partly filled as shown in FIG. 4.3.2.2A. The highest energy attained by electrons in this band is called the Fermi energy or Fermi level.

Semiconductors have a similar band picture to insulators except that the separation of the empty and filled energy bands is small, as is shown in FIG. 4.3.2.3. The band gap must be such that some electrons have enough energy to be transferred from the top of the valence band to the bottom of the conduction band at room temperature. Each electron transferred will leave behind a “vacancy” in the valence band. Rather surprisingly, these vacancies behave as if they were positively charge electrons. They are known as positive holes, or just holes. Therefore, each time an electron is removed from the valence band to the conduction band two mobile species are created, an electron and a hole.

Light may be considered to consist of photons that can be emitted, reflected, transmitted and absorbed. Photons normally travel in straight lines until they hit a surface. The interaction between photons and surfaces is twofold: first, photons may be absorbed by the surface, where they are converted into thermal energy or, second, they may be reflected in some direction. The distribution of reflected directions, given an angle of incidence, gives rise to a surface's appearance.

The model of band energies explains why most dielectrics are transparent and why metals and semiconductors are opaque. It also explains why materials have different colors depending on whether the material is an insulator, a semiconductor, or a metal.

The energy photons in the visible region of 400-700 nm have an energy range of 3.0 eV to 1.8 eV. In dielectrics the band gap has an energy of several electron volts. In the particular case of high purity silica, a common glass used in windows whose composition is very close to SiO₂, the band gap is about 9 eV. A photon of at least 137 nm is necessary to transfer an electron from the top of the valence band to the bottom of the conduction band. 137 nm is in the VUV (ultraviolet) region, very far from the visible region (see FIG. 4.3.3.1Aa).

This means that the photons of visible energy do not alter the electrons of the valence band, and the conduction band stays altered. NO photons of visible light (from the short-wavelength section of the spectrum to the long-wavelength section of the spectrum) alter the electrons of an insulator and therefore, ALL photons of visible light are transmitted through the material. Visible light, therefore, is not modified by this material because there is almost no interaction with its electrons. This is the reason that many insulators appear transparent to our eyes (see FIG. 4.3.3.1Ab)

However, when insulators or dielectrics have “impurities,” meaning other electric components within the material, they can become translucent and/or colored. Photons having energies lower than 9 eV, meaning photons with energy that correspond to the visible spectrum, can be absorbed by electrons and then converted into heat. Light appears to be white when all the photons that describe a wavelength within the visible spectrum are present (from the short-wavelength violets to the long-wavelength reds). If one of the photons corresponding to a determined wavelength is absorbed, the spectrum is “incomplete” and the resulting light that has interacted with the material will appear colored. The resulting color depends on the specific impurities (see FIG. 4.3.3.1B). This phenomenon can be used to make glass with beautiful colors, depending on the wavelengths absorbed. If the impurities in silica absorb all the wavelengths of the visible spectrum, then silica appears black (i.e., obsidian).

As explained above, the conduction band of metals is partially filled with electrons. The higher electronic energy level of this conduction band, partially filled with electrons is called the Fermi level. Above the Fermi energy level almost all the levels are empty and available to accept excited electrons. The excited electrons that will fill these energy levels above the Fermi level will come from the energy levels below the Fermi level. All incident radiation can be absorbed, irrespective of its wavelength, because there is always an empty level available to accept the excited electron.

The electrons under the Fermi level are excited when they interact with a photon of a light beam (see point 1 of FIG. 4.3.3.2A). The electrons get “excited” because they acquire the energy of the photon and become transferred to empty energy levels above the Fermi level (see point 2 of FIG. 4.3.3.2A). We say they get “excited” because these electrons take the energy of the photon and start oscillating (see point 3 of FIG. 4.3.3.2A). As they oscillate, they reradiate energy. When they stop oscillating, they immediately fall back to their original level (see point 4 of FIG. 4.3.3.2A), and by falling back, they emit a photon with almost the same energy of the original photon (see point 5 of FIG. 4.3.3.2A), giving metals their highly reflective appearance.

A semiconductor's atomic behavior is somewhere between a metal and an insulator, and its color is also governed by the energy in its band gap. When the energy gap is relatively large, light photons are not energetic enough to excite an electron from the valence band to the conduction band, and so they are not absorbed. The material will appear transparent to these wavelengths. On the other hand, if the minimum energy is quite small and lies in the infrared region, the semiconductor will absorb (and reflect) the entire visible spectrum and take on a metallic appearance. If the band gap falls in the visible spectrum, the semiconductor will absorb all photons with an energy greater than the band gap, but not those with a smaller energy. This will cause the material to be strongly colored. Like dielectrics, impurities in semiconductors can change their electronic configuration, altering the energies of photons that can be absorbed and giving semiconductors different colors.

In the experiments described below, one focus is on the behavior of insulators and metals, considering that semiconductors behave somewhere in between.

MACROSCOPIC INTERACTION BETWEEN LIGHT AND MATTER. It is easier to explain the processes of reflection, transmission, refraction, absorption and scattering from a macroscopic point of view—in other words, at a wavelength level. Macroscopic interactions of light with matter produce the color and light intensity of objects detected by the eye. These phenomena depend on wavelength of light (λ) and on the index refraction, which in turn depends on material characteristics.

Light can interact with a transparent material in several ways, shown schematically in FIG. 4.4A. The incident light can be reflected from any surface. The appearance of a solid is often dominated by reflection. Light white passing through the material can be scattered or absorbed. When some of the absorbed light is re-emitted, usually at a lower energy, it is called fluorescence. The light that leaves the material is transmitted light.

Reflection, absorption, and scattering all give rise to the world of light color and intensity that the eye perceives. Certain embodiments described below concentrate on reflection, scattering, and absorption: these three phenomena determine the spectrum and intensity properties of the light “coming out” of a material.

First, reflection. When a light ray hits a surface, it bounces at the same angle with which it hit the normal face of the surface. In other words, the angle of incidence equals the angle of reflection. The plane of incidence contains the incident ray, the reflected ray, and the normal to the reflecting surface (see FIG. 4.4.1.1A).

Reflection can be specular or diffusive. If the surface of the material is rough, light is reflected diffusively, or in other words, with an erratic dispersion (FIG. 4.4.1.1Ba). This happens when the “mountains” and “valleys” of the surface are larger than the length of the wave of the light beam (in other words, the surface is too rough for the length of the wave). If the surface is smooth the reflection is said to be specular: light is reflected coherently (FIG. 4.4.1.1Bb). As a light beam goes down to a horizontal, smooth surface (i.e., a mirror) it is reflected in a perfectly symmetrical way. This ideal reflectance occurs when any irregularities present on a surface are smaller than the length of the wave of the light beam. Then, incident light regards the surface as smooth and reflects coherently. If the surface is semi smooth, a combination of both types of reflection are present (FIG. 4.4.1.1Bc).

The diffuse reflection component increases with surface roughness at the expense of the specular component so that a finely ground powder surface shows only diffuse reflection. The gloss of a surface is a measure of the relative amounts of diffuse reflectance to specular reflectance. Glossy surfaces have a large specular component.

Matte surfaces distribute light almost evenly in all directions (FIG. 4.4.1.1Ba), whereas glossy and shiny surfaces reflect light in a preferred direction. This causes highlights that may be as strong as the original light sources (FIG. 4.4.1.1Bb).

The coefficient of reflection is defined such that if a wave of amplitude a0 falls upon the surface, then the amplitude of the reflected wave is rao. For reflection at a surface between a substance of low refractive index and a substance of high refractive index, r is negative. This signifies a phase change of π radians on reflection, which means, in terms of the light wave, that a peak turns into a trough and vice versa, as illustrated in FIG. 4.4.1.1C.

Second, absorption. Color due to absorption is caused by the fact that some of the incident wavelengths are more strongly attenuated than others. Absorption of some wavelengths results from the presence of absorption centers within the material, including dye molecules, transition metal ions, or small metal particles. When absorption centers are distributed uniformly throughout the bulk of the material, the amount of light absorbed is given by: I=Iₒ exp (−αₛl). where “Iₒ” is the incident beam intensity; “I” the intensity after traveling a distance “l” in the turbid medium; and “αₛ” is an experimentally determined linear absorption coefficient.

The degree of absorption varies significantly across the visible spectrum and many absorption centers show a pronounced maximum absorption at a particular wavelength. The amount of absorption will undoubtedly be a function of the concentration of the absorbing centers throughout the bulk of the material.

DIFFERENCE BETWEEN METALS AND OPAQUE INSULATORS: REFLECTION, ABSORPTION, AND SCATTERING. Reflection, absorption, and scattering are macroscopic phenomena that differ between metals and insulators because of varying atomic configurations described in the section about the interaction between light and matter at a microscopic level.

The color and intensity of an insulator result from ratios of reflection and absorption: light impinging on the surface is partially reflected and the remainder is absorbed.

In the first case, light hits the surface and only certain wavelengths reflect, changing the color of the original light source. However, because light doesn't penetrate the material, the spectral component (color) of the resulting light is not going to change much from the color of the original light beam. The more specular the reflection is, the more evident. For instance, when we look at a red glossy sphere, the highlight (concentrated reflection of the light source due to specular reflection) looks white if the original light source is white (see FIG. 4.4.2.1Aa)

The same thing would happen with a glossy black sphere. We would expect that the object would look completely black due to the fact that all the frequencies of the visible range are absorbed. But if the sphere is highly glossy, a reflection will occur and again the highlight will mostly resemble the spectrum of the light source (see FIG. 4.4.2.1Ab)

In the second case, as the source light beam enters into the middle of the visible spectrum, a selected part of the spectrum is absorbed and dissipated in the material as heat (depending on the characteristics of the material's absorption centers). The rest of the spectrum is not absorbed and therefore remains visible to the eye with a different resulting spectrum and intensity. In the same example of the red sphere, the absorption centers would remove the blues and greens from the spectrum (short-wavelengths) and the reds (long-wavelengths) would remain visible to the eye; the reds are reflected visible to the eye (see FIG. 4.4.2.1Ba).

In the case of the black sphere, all the visible frequencies are absorbed and none remain visible to the eye; none are reflected visible to the eye (see FIG. 4.4.2.1Bb).

Reflectance is the term used to describe the percentage of the visible spectrum that is reflected from the object, which remains the same no matter what the illumination. For example, the red sphere is red no matter the spectrum and intensity of the original light source because the material will always absorb the same percentage of blues and greens and reflect the same percentage of reds.

COLOR AND INTENSITY IN METALS. Color and intensity of a metal is given mainly by reflection and remittance. Light impinging on the surface is partially reflected when the surface is very smooth, but most of it is reemitted back.

In the first case, reflection, the phenomena is the same as the insulators. However, the spectrum and intensity of the resulting light will partially acquire the spectral and intensity properties of the original light source. This is because remittance is so strong, that both phenomena are combined and perceived by the eye (FIG. 4.4.2.2Aa). The second case, scattering (remittance) is very different. In the section about the interaction between light and matter at a microscopic level, it was explained how electrons under the Fermi level are excited by taking energy from a photon. It was also explained how this oscillation causes light to be reemitted. Light remittance is spectral selective: some wavelengths are reemitted more than others. This effect of the electrons taking energy and then remitting makes metals appear to be highly reflective even though their surface may not be smooth (FIG. 4.4.2.2Ab).

In short, the color of an insulator is given by preferential spectral absorption; and the spectral properties and intensity of light due to specular reflection are going to be very similar to those of the original light source. On the other hand, the color of a metal is given mainly by preferential spectral remittance; while the spectral properties and intensity of light due to specular reflection are going to be modified by the color of the metal per se.

FIG. 4.4.2.2B is a computer generated image done with the software RADIANCE (discussed further below) that shows some examples of how the highlights of different colored spheres of insulator (left row) and metallic material (right row) appear under different light sources of different spectral composition.

### Radiance: the Program

RADIANCE is a professional toolkit for visualizing and calculating light systems in virtual environments. RADIANCE is not photorealistic rendering software that aims to imitate photographic images, but rather it is a lighting visualization and calculation program that works with physical units. Photorealistic rendering programs use digital “tricks” to imitate and approximate the visual effects of light reflected off of materials; such programs attempt to reproduce reality, but are lacking any physical basis in reality.

RADIANCE, on the other hand, performs its computation in radiance, or radiant existent units (radiosity). Also, the local illumination model adheres to physical reality; it therefore accurately describes the way light is emitted, reflected, and transmitted by each surface in the digital model.

RADIANCE's 4 main capabilities: accurate calculates luminance and radiance; models both electric light and daylight; supports a variety of reflectance models; and supports complicated geometries.

The description of the program below is based on the book titled Rendering With Radiance: The Art and Science of Lighting Visualization by Greg Ward and Rob Shakespeare (Ward and Shakespeare, 1998).

ACCURACY OF THE PROGRAM. As explained before, luminance is the photometric unit that is best correlated with what the human eye sees. Radiance is the radiometric equivalent of luminance, and is expressed in SI (Standard International) units of watts/steradian/m². RADIANCE (the software) endeavors to produce accurate predictions of these values in modeled environments, and in so doing permits the calculation of other, derived metrics (for all metrics are derivable from this basic quantity) as well as synthetic images (renderings).

Since RADIANCE is designed for general lighting prediction, it includes all important sources of illumination. For architectural spaces, the two critical sources are electric light and daylight. Modeling electric light accurately means using measured and/or calculated output distribution data for light fixtures (luminaires). Modeling daylight accurately means following the initial intense radiation from the sun and redistributing it through its various reflections from other surfaces and scattering from the sky.

FIG. 5.1A shows a RADIANCE rendering of a conference room at Berkeley Laboratory (model created by Anat Grynberg and Greg Ward). The model for this room was derived by measuring the dimensions of the real space and furnishings shown in FIG. 5.1A. The similarity between the two images testifies to the accuracy of the luminance calculation, even if no numeric values are shown. The first image corresponds to a RADIANCE render and the second to a real photo of the room.

FIG. 5.1B shows a comparison between measured illuminance values under daylight conditions and RADIANCE predictions based on simultaneous measurements of the sun and sky components (March 1995). This attests to the numerical accuracy of the daylight calculation in RADIANCE.

RAY-TRACING METHOD IN RADIANCE. Many lighting visualization programs are based on the radiosity method which typically models surfaces as ideal Lambertian diffusers. A Lambertian diffuser refers to those surfaces that reflect light in a perfectly diffuse and uniform fashion. This is at best a gross simplification, but it is a very convenient one to make (computationally speaking). The best methods include specular and directional-diffuse reflection, as used in RADIANCE. Most important, local illumination models must include an accurate simulation of emission from light sources; if the source is not rendered correctly, no following computations will save the result.

RADIANCE employs a light-backwards ray-tracing method (see appendix for information about the ray-tracing method), extended from the original algorithm introduced to computer graphics by Whitted in 1980 (Larson, Shakespeare, 1998). Light is followed along geometric rays from the point of measurement (the view point or virtual photometer) into the scene and the back to the light sources. The result is mathematically equivalent to the following light forward, but the process is generally more efficient because most of the light leaving a source never reaches the point of interest.

The difficulty of light-backwards ray tracing as practiced by most rendering software is that it is an incomplete model of light interaction. In particular the original algorithm fails for diffuse inter-reflection between objects, which it usually approximates as a constant “ambient” term in the illumination equation. Without a complete computation of global illumination, a rendering method cannot produce accurate values and is therefore of limited use as a predictive tool. RADIANCE overcomes this shortcoming with an efficient algorithm for computing and caching indirect irradiance values over surfaces, while also providing more accurate and realistic light sources and surface materials.

SCENE DESCRIPTIONS. The representation of the materials is very important because it determines how light interacts with surfaces and their specific properties. Version 3.1 of RADIANCE, used herein, includes 25 material types. Some most common material examples are as follows.

Light: Light is considered an emitting surface, and it is by material type that RADIANCE determines which surfaces act as light sources. Lights are usually visible in a rendering, as opposed to many systems that employ non-physical sources, and then “hide” the evidence. Lights are pure emitters; they do not reflect.

Illum: Ilum is a special light type for secondary sources, sometimes called impostors. An example of a secondary source is a window through which natural light enters a room. Since it is more efficient for the calculation to search for light sources, marking a window as an illum can improve rendering quality without adding to computation time.

Plastic: Opaque, non-metallic materials. Despite its artificial sounding name, most materials fall into this category. A plastic surface has a color associated with diffusely reflected radiation, but the specular component is uncolored. This type is used for materials such as plastic, painted surfaces, wood, and nonmetallic rock.

Metal: Metallic materials. Metal is exactly the same as plastic, except that the specular component is modified by the material color.

Dielectric: A dielectric surface refracts and reflects radiation and is transparent. Common dielectric materials include glass, water, and crystals. A thin glass surface is best represented using the glass type, which computes multiple internal reflections without tracing rays, thus saving significant rendering time without compromising accuracy.

Trans: A trans material transmits and reflects light with both diffuse and specular components going in each direction. This type is appropriate for thin translucent materials.

BRTDfunc: This is the most general programmable material, providing inputs for pure specular, directional diffuse, and diffuse reflection and transmission. Each component has an associated (programmable) color, and reflectance may differ when seen from each side of the surface.

Most other material types are variations on those listed above, some using data or functions to modify the directional-diffuse component. Certain aspects of the invention relate to the use of plastic (opaque, non-metallic materials) and metal (metallic materials) for the surfaces, and light (a diffusive artificial light source).

MATERIAL DESCRIPTIVE PARAMETERS. Each material type uses a specific set of parameters.

Light is a self luminous surface and it can be used to describe simple diffused artificial light sources. This material is defined simply as an RGB (red, green, and blue) radiant energy emitter. The RGB parameters are in physical units of radiance value (watts/sr/m²).

Plastic is used for most everyday opaque, non-metallic materials. Plastic is a material with uncolored highlights (or to be more specific, plastic materials acquire the color of the light source). This is based on the interaction of light and insulators described herein. Plastics can be described by the following parameters. (1) RGB (red, green and blue) reflectance: Reflectance refers to the percentage of light reflected from the object, which remains the same no matter what the illumination. A value equal to 1 corresponds to 100% and 0 to 0%. In RADIANCE, values greater than 0.9 for plastic materials are usually not realistic. (2) Fraction of specularity: This fraction refers to the smoothness of a surface. It gives an appearance of “glossiness” to the material, and therefore determines how specular the reflection is going to be (in other words, it determines reflectivity). Specularity fractions greater than 0.1 for plastic materials are not very realistic. (3) Fraction of roughness value: Roughness is specified as the rms (root mean square) slope of surface facets. Roughness varies from 0 (perfectly smooth) to 0.2 (perfectly rough). Roughness greater than 0.2 in plastics is usually not realistic. FIG. 5.3.2A presents some examples on how a plastic material would look like when combining different values of specularity and roughness. It represents renders of blue spheres with different specularity and roughness values reflecting white light from a single source.

Metal is used to describe metallic materials. In RADIANCE it is similar to plastic, but in metals, specular highlights are modified by the material color. This is based on the interaction of light and metals as described herein. Metals can be described by the following parameters: (1) RGB (red, green and blue) reflectance: Reflectance refers to the percentage of light reflected from the object, which remains the same no matter what the illumination. A value equal to 1 corresponds to 100% and 0 to 0%. In RADIANCE, values greater than 0.9 in metals are not usually realistic. (2) Fraction of specularity: This fraction refers to the smoothness of a surface. It gives an appearance of “glossiness” to the material, and therefore determines how specular the reflection is going to be; (in other words, it determines reflectivity). Specularity fractions greater than 0.9 are common in metals (because of its high reflectivity). (3) Fraction of roughness value: Roughness is specified as the rms (root mean square) slope of surface facets. Roughness varies from 0 (perfectly smooth) to 0.2 (perfectly rough). Roughness greater than 0.2 is usually not realistic in metals.

Textures vary the apparent local shape of a surface by perturbing the surface normal across the object surface, which causes shading variations perceived as bumps, waves, etc. In RADIANCE textures can be described as mathematical procedures.

### An Architectonic Approach to Lighting for the Circadian System

Today, spectral and intensity characteristics of light delivered by indoor illumination systems are not proper to optimally suppress melatonin. This fact has translated into poor performance and lack of alertness, psychophysical disorders such as depression and insomnia, and even serious health problems such as cancer (more specifically, breast cancer). Therefore, light characteristics in indoor spaces must change to enhance a “healthier” and more proper function of the human circadian system.

In indoor spaces the use of monochromatic light is not recommendable because it can create visual discomfort and problems concerning visual clarity, luminance contrast, and color difference. Therefore, for the visual system, light sources are herein recommended that present a spectrum that includes different frequencies. Further, different intensity levels are recommended for the visual system depending on different tasks.

To complicate matters more, the spectrum and intensity of light that is optimal for the performance of the circadian system is different from that of the visual system. Indoor illumination must be designed for the optimal stimulation of both the visual and circadian system. However, the light characteristics designed to serve the visual system should not cause an improper functioning of the circadian system; and inversely, the proposed light design meant to enhance the proper functioning of the circadian system must not cause visual discomfort. Unfortunately, it is not possible to create one illumination system that can deliver the optimal light characteristics for both systems at the same time. While the lighting industry has proven that illumination systems can be optimized for visual comfort, herein it is proposed that an architectonic approach to lighting design can be incorporated so that a proposed lighting system will also deliver adequate spectral and intensity characteristics of light for the circadian system.

Herein it is demonstrated that it is possible to enhance the intensity and specific parts of the spectrum by combining different spectral and intensity characteristics of a light source with different reflectance indexes, specular values, and textures of surfaces. This technique demonstrates a passive, architectonic approach to enhancing the spectrum and intensity characteristics of a pre-existing, or generic illumination system.

Until now, previous research has shown that the most proper light for melatonin suppression is blue monochromatic light (between about 460 nm and about 480 nm) at a high level of intensity. These light characteristics may be needed in indoor spaces where high levels of alertness and performance are important. Bright blue monochromatic light, however, would cause visual discomfort by, for example, creating glare and problems in luminance and color contrasts. Additionally, high intensity levels of light in an indoor space may create other problems such as high energy consumption in the building.

Therefore, one aspect of the invention relates to a light source which is non-monochromatic. In certain embodiments, the light source is richer in the blue part of the spectrum. In certain embodiments, the intensity of the light source was fixed at 3500 watts.

The results of the light simulations described herein demonstrated that the blue part of the spectrum of a light source can be maximized by using materials which comprise non-metallic materials (insulators) and have a texture that would concentrate the intensity of the blue spectrum towards a specific point in space. Although metals are highly reflective, the ratio of intensity of the blue spectrum reflected off of a flat versus textured wall is not as great as in an insulator.

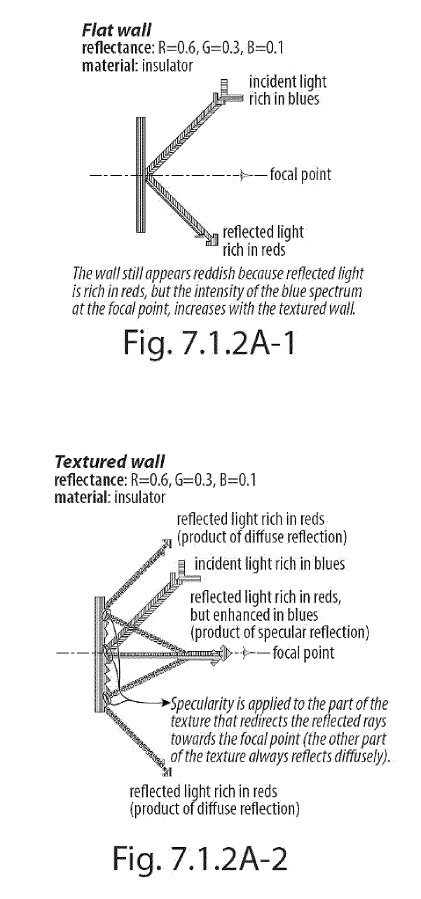

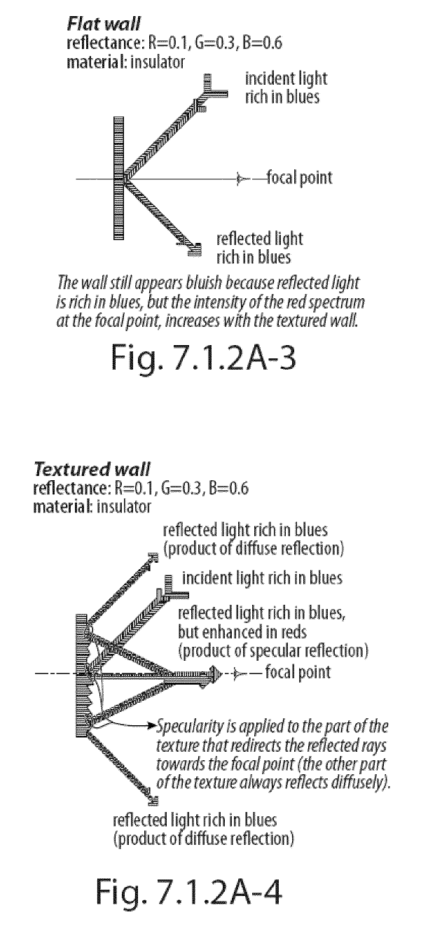

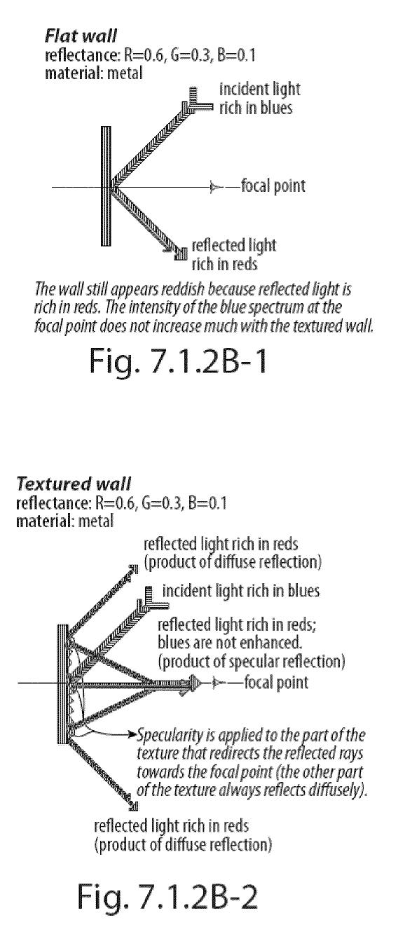

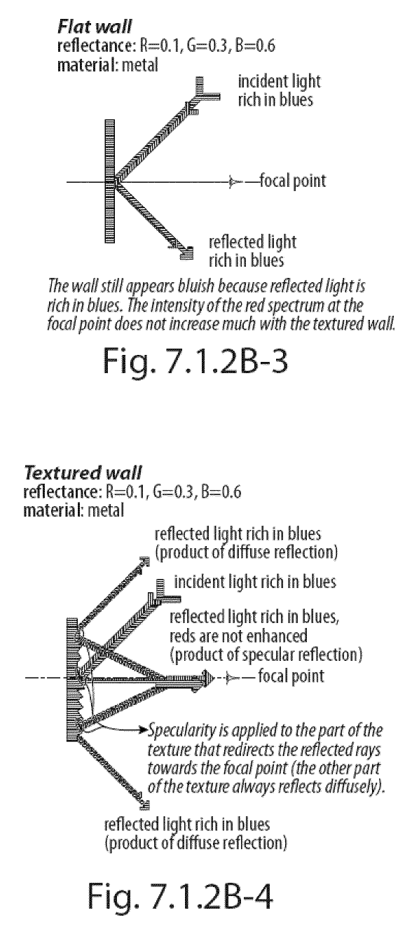

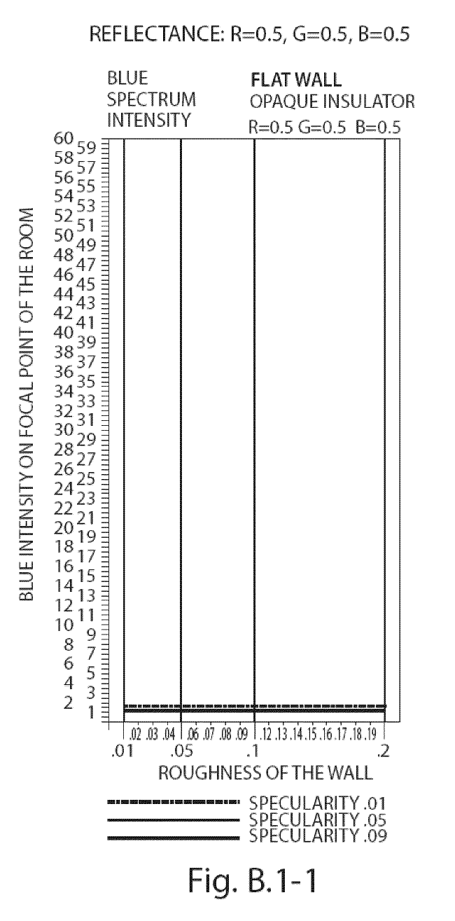

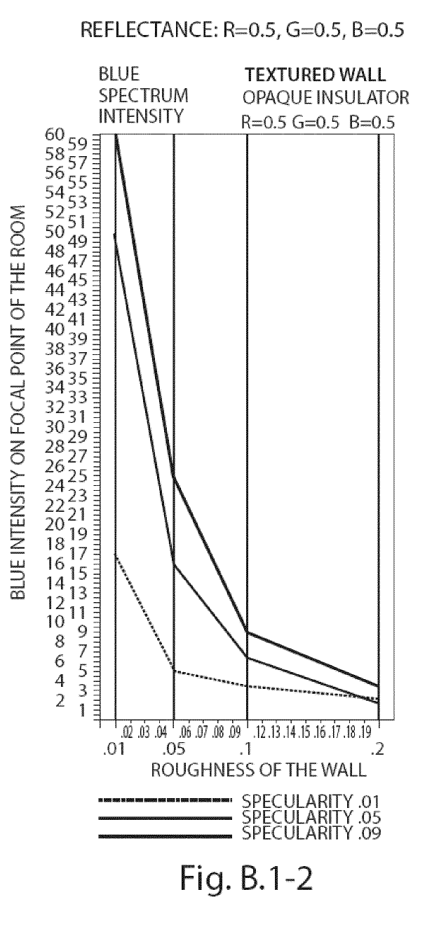

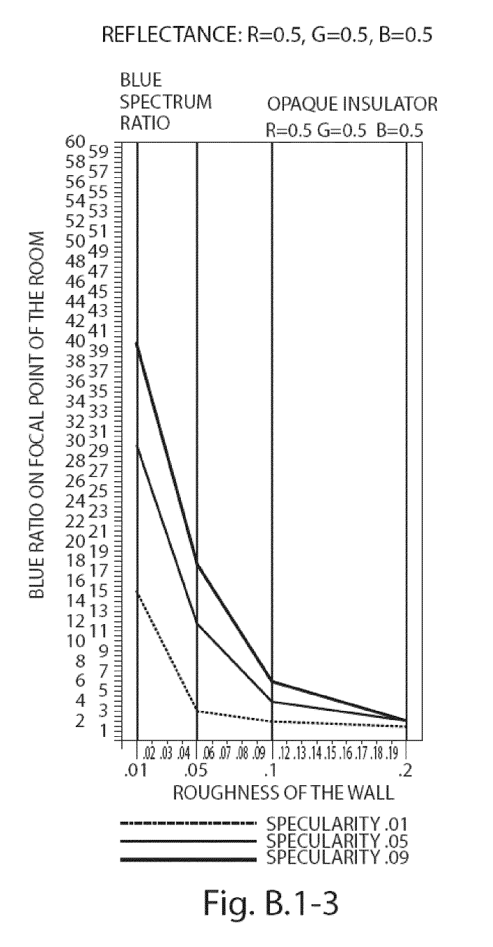

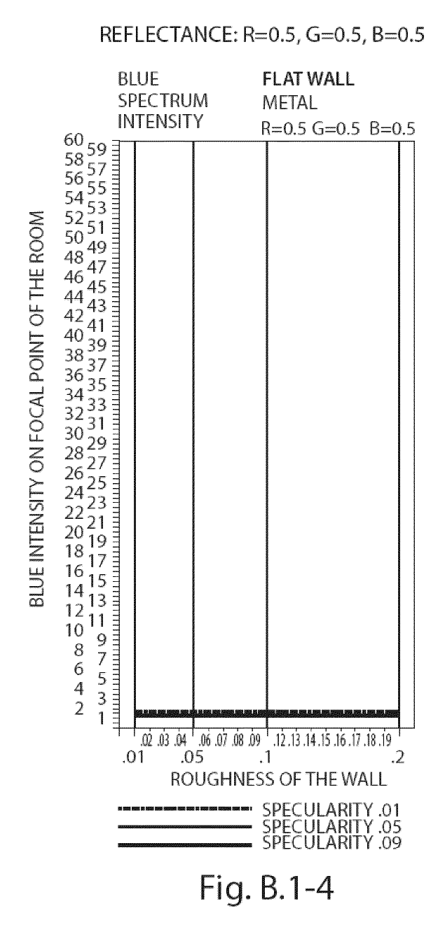

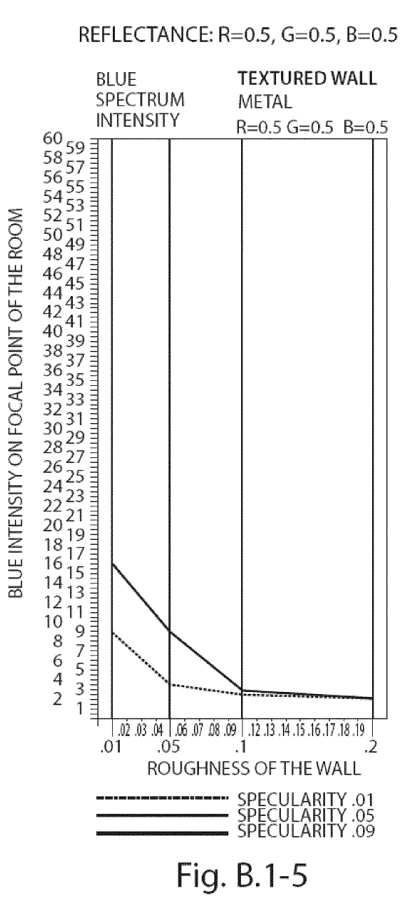

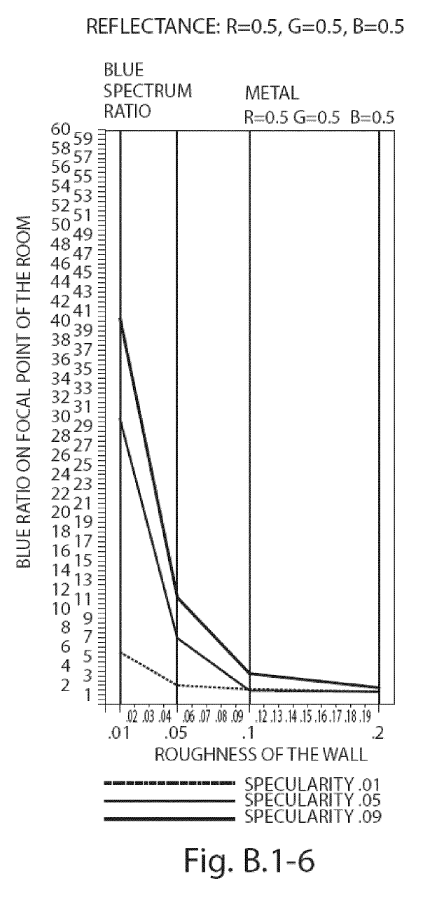

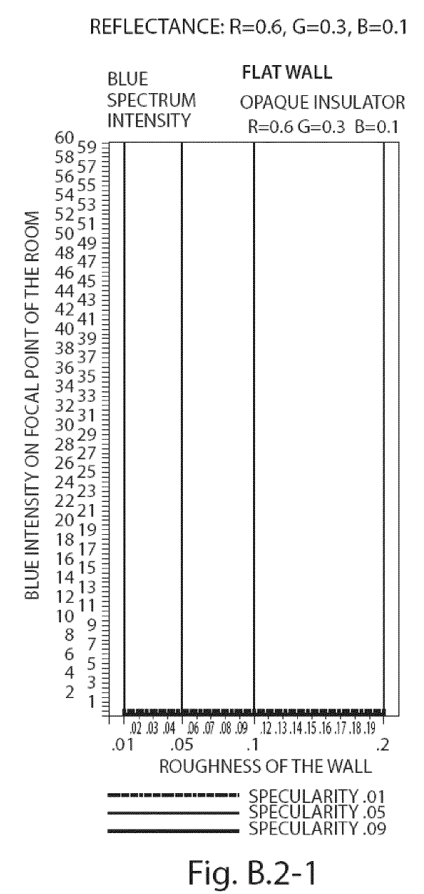

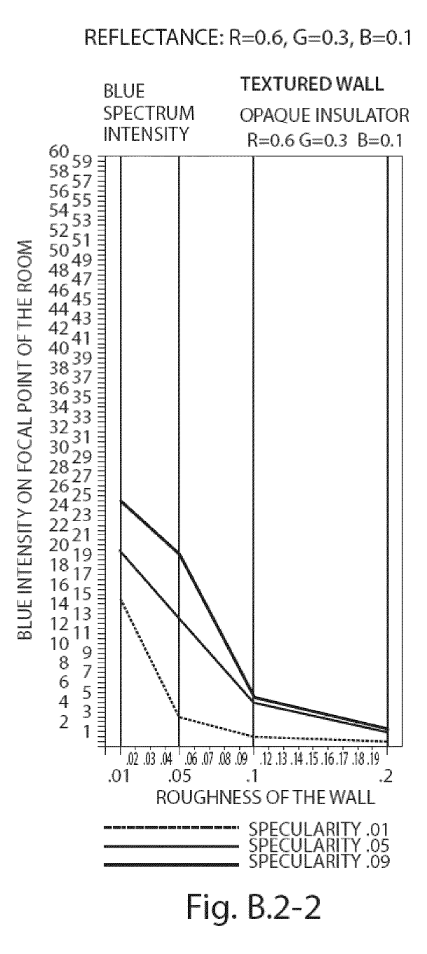

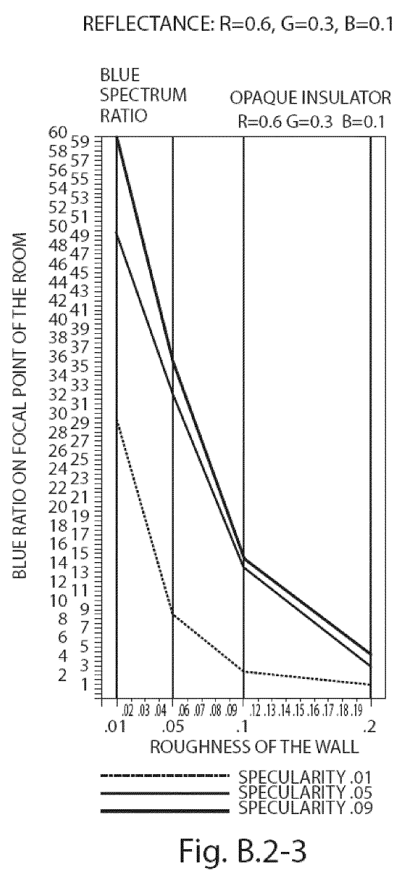

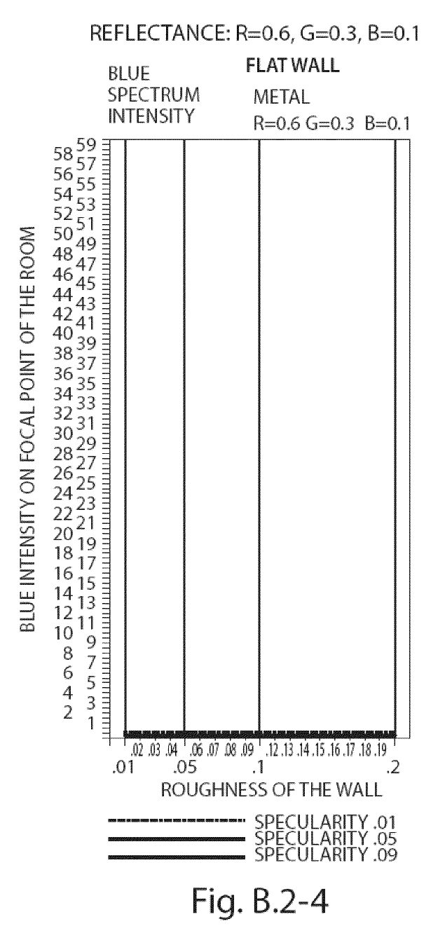

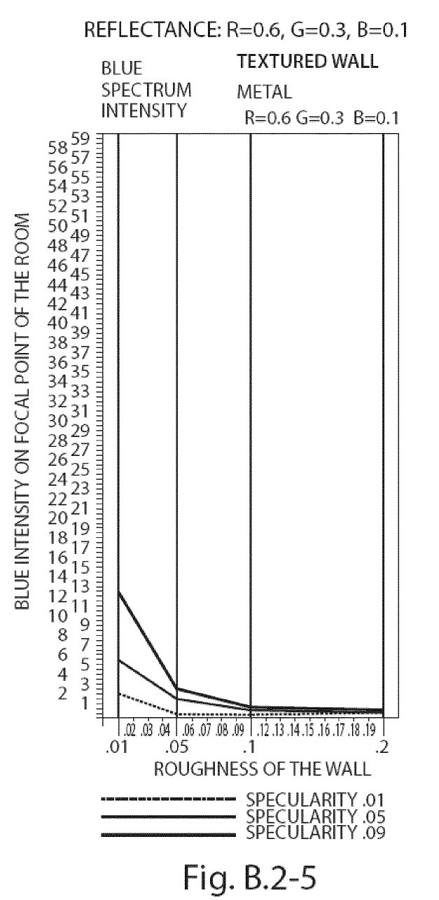

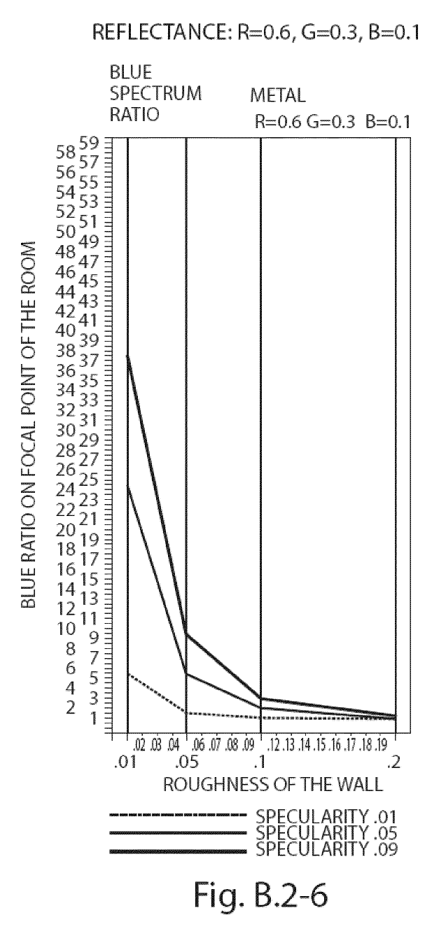

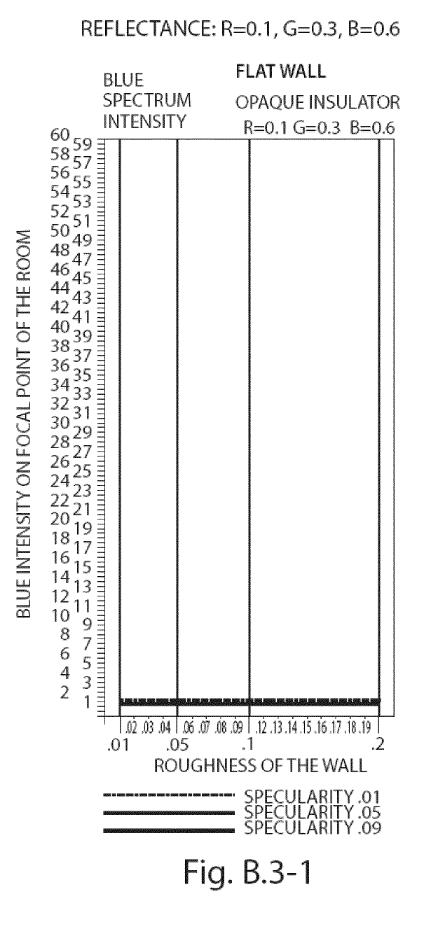

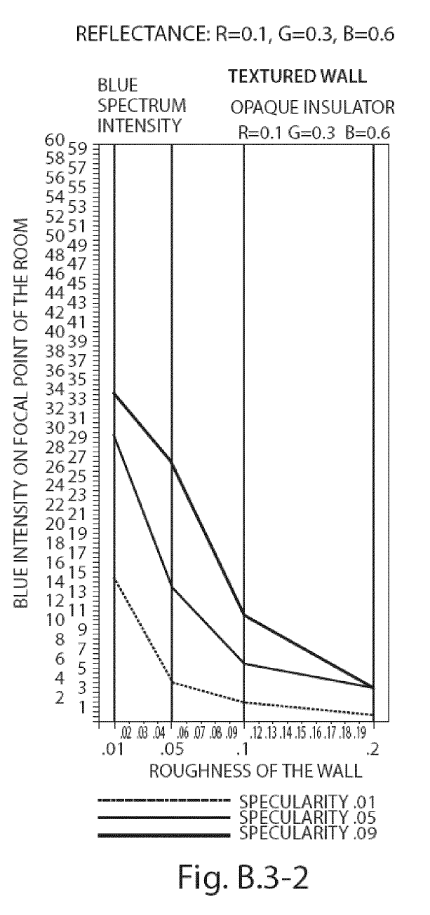

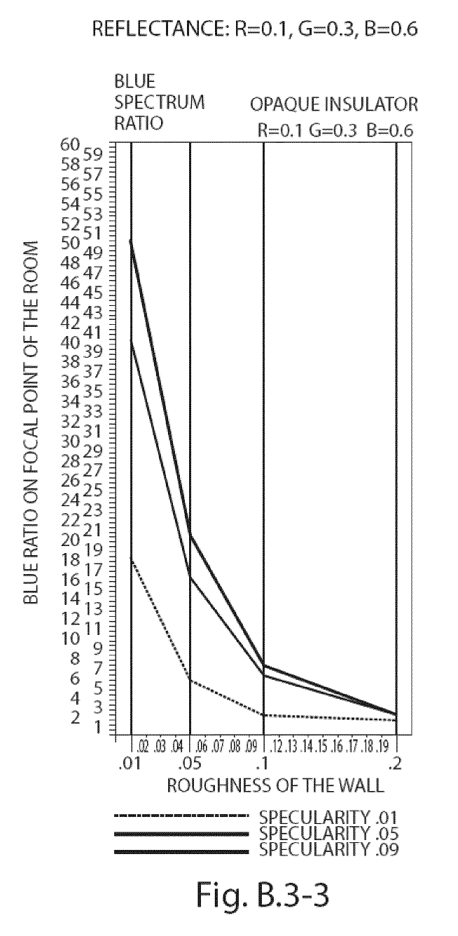

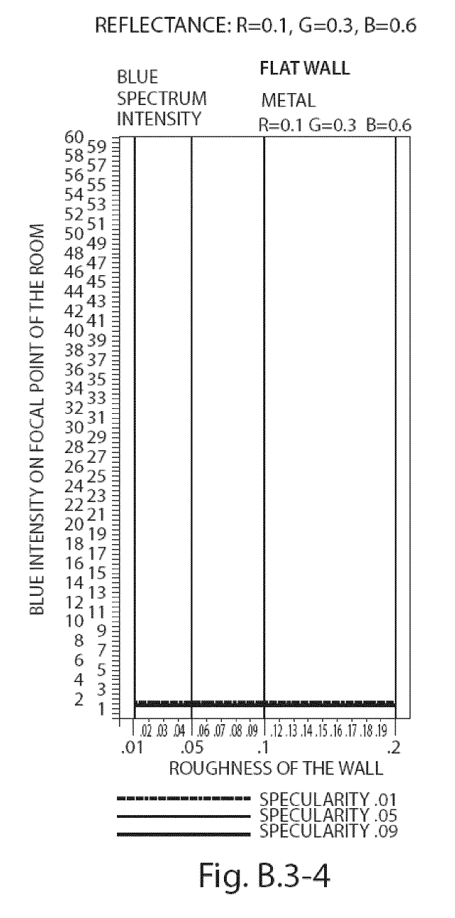

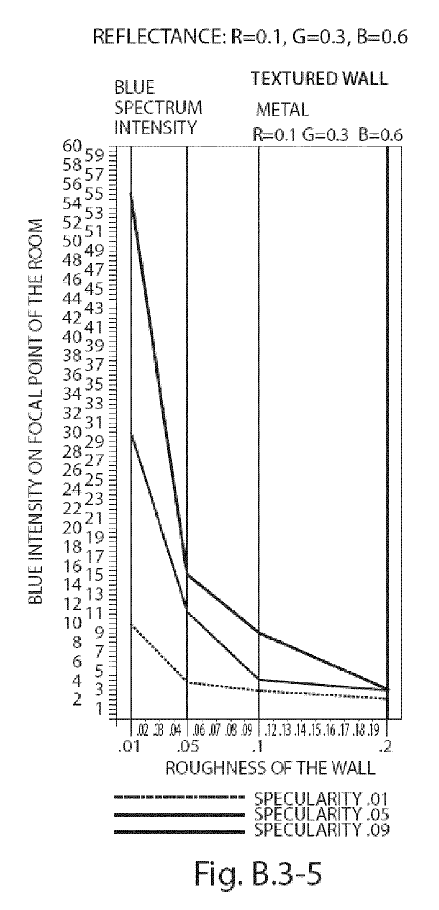

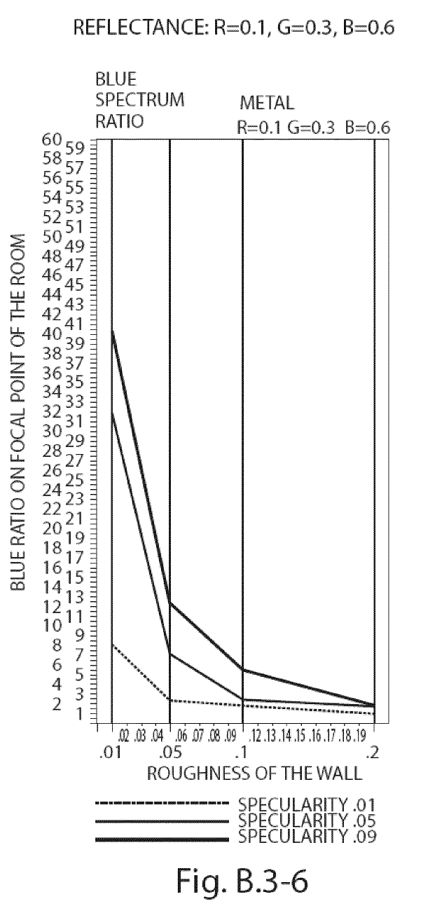

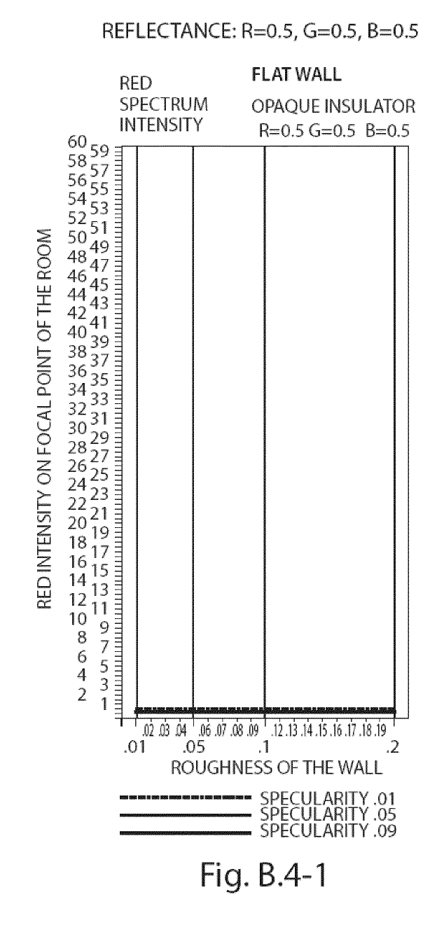

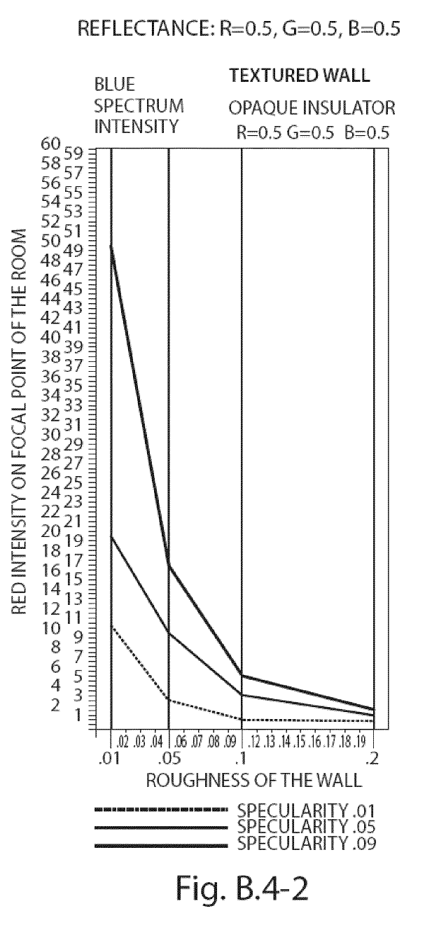

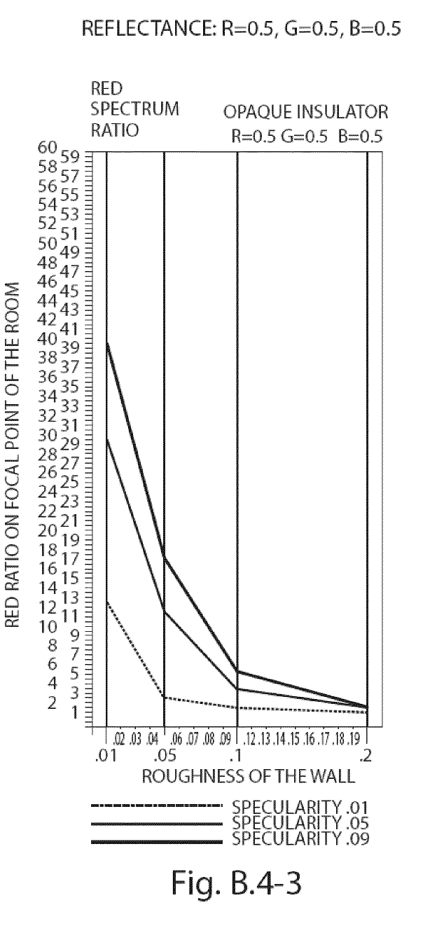

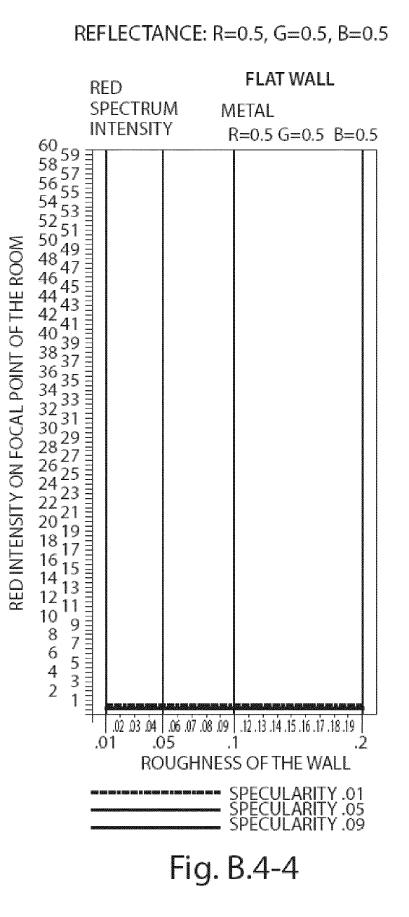

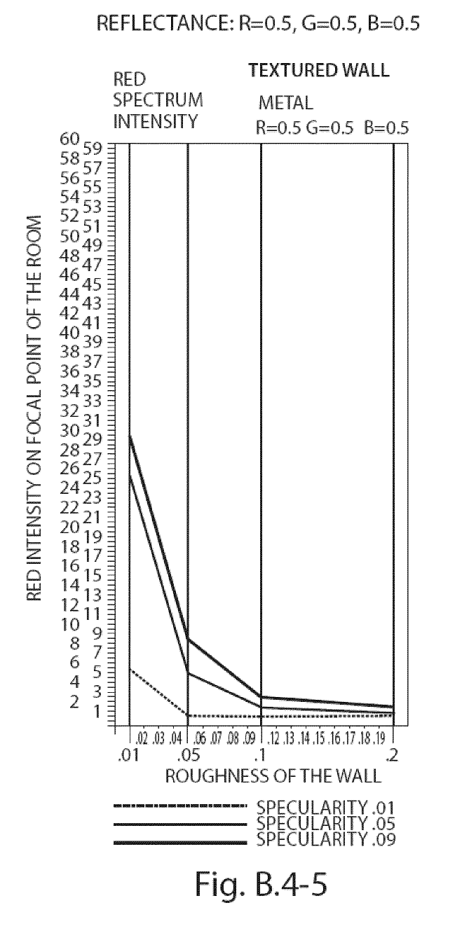

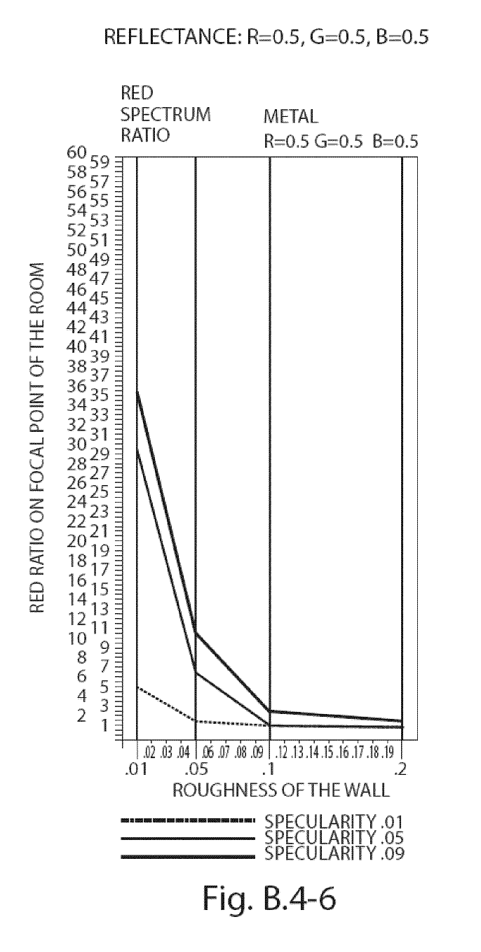

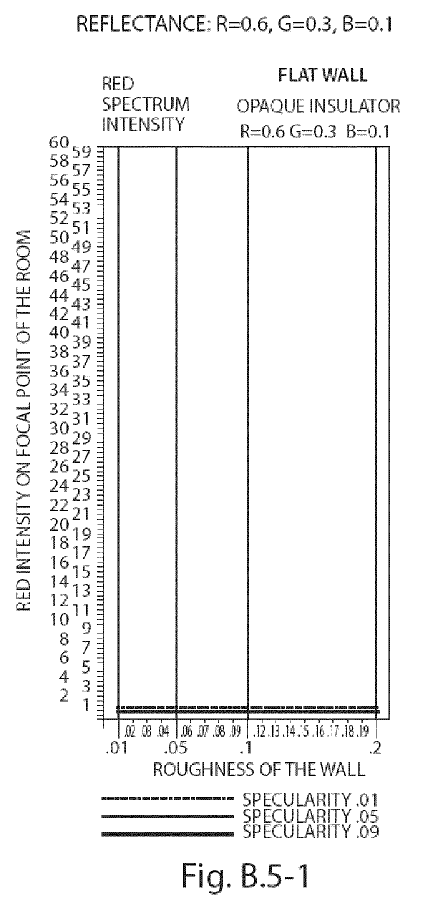

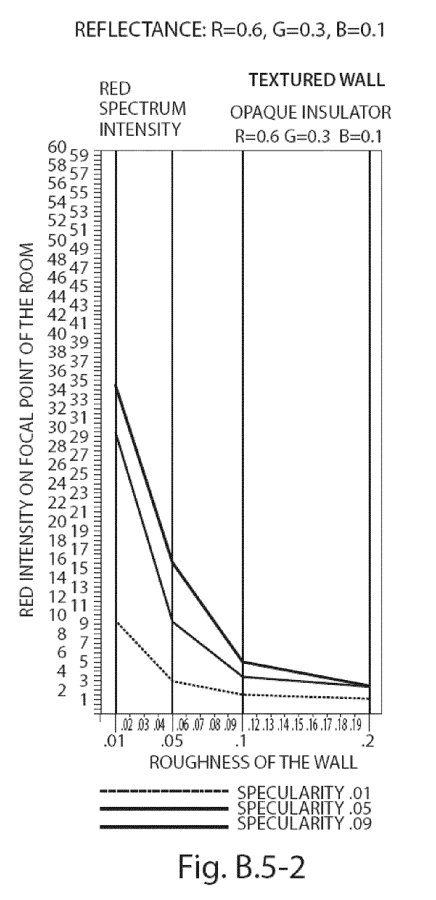

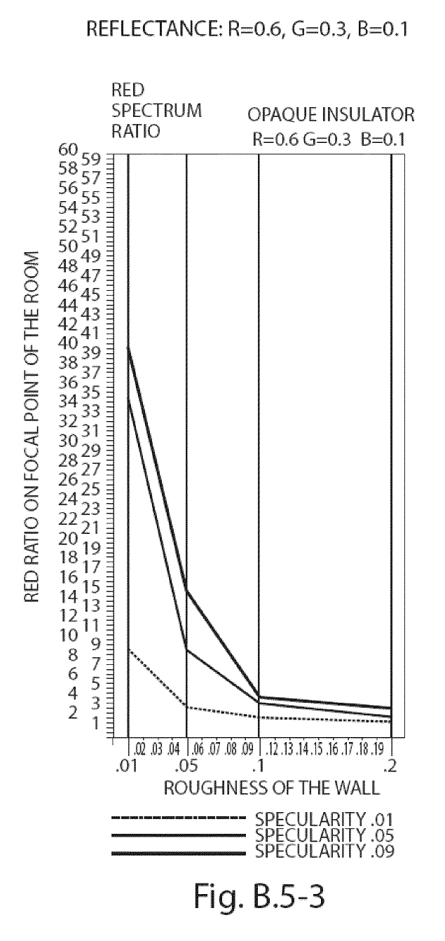

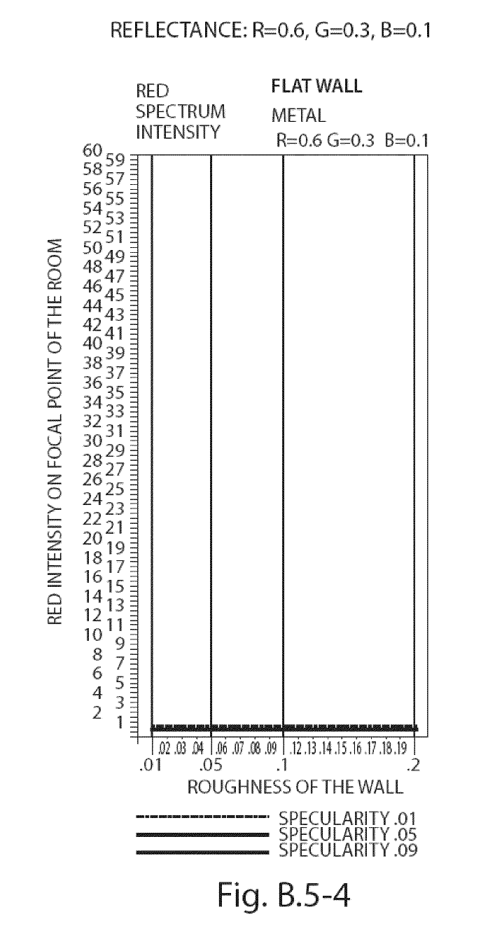

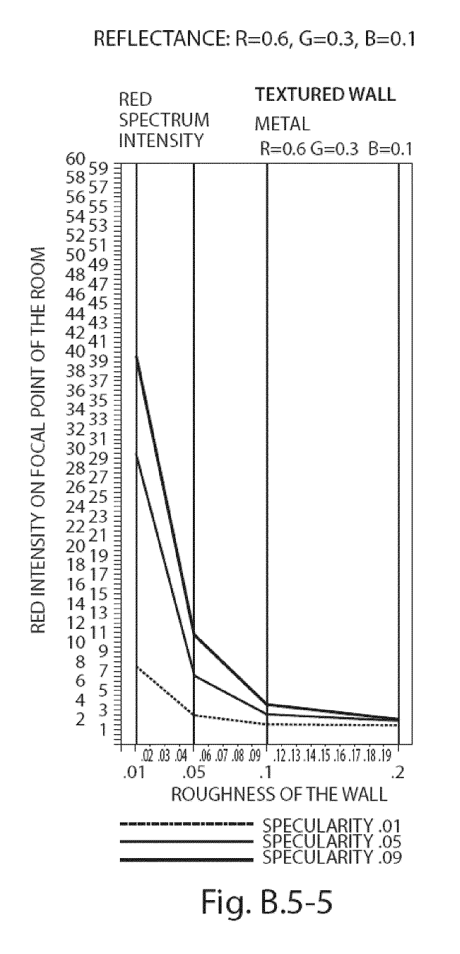

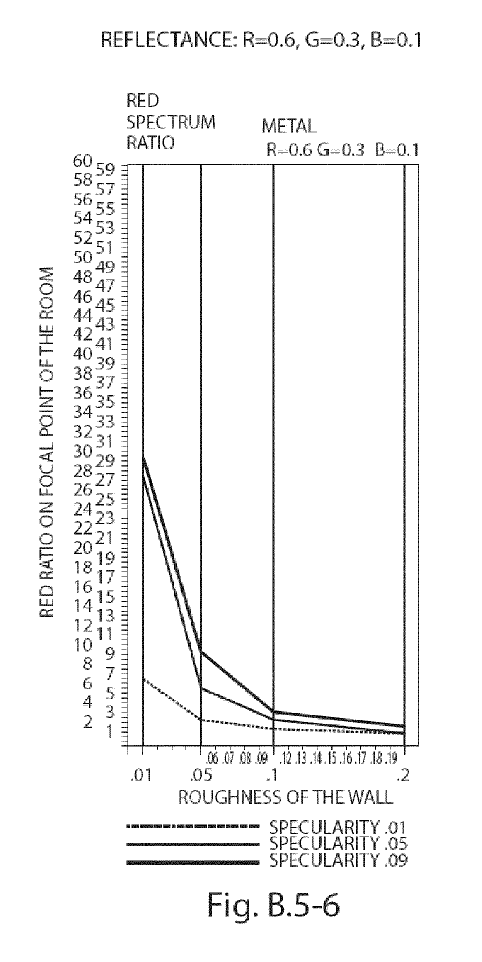

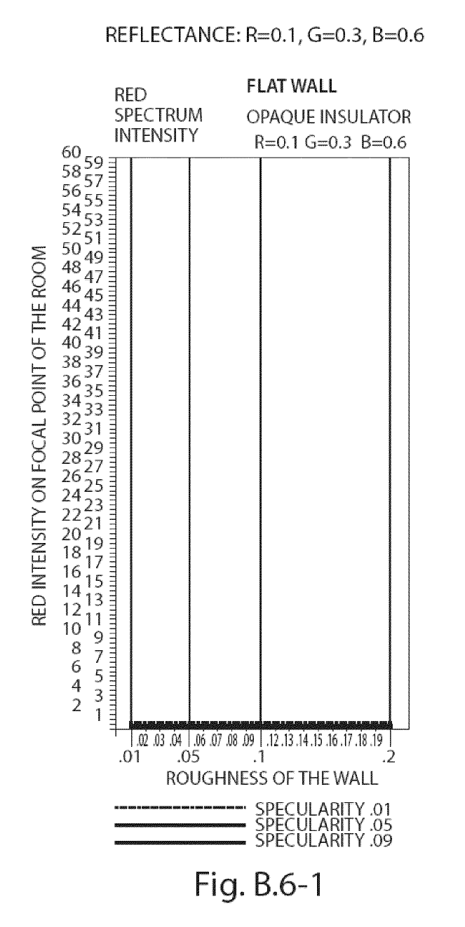

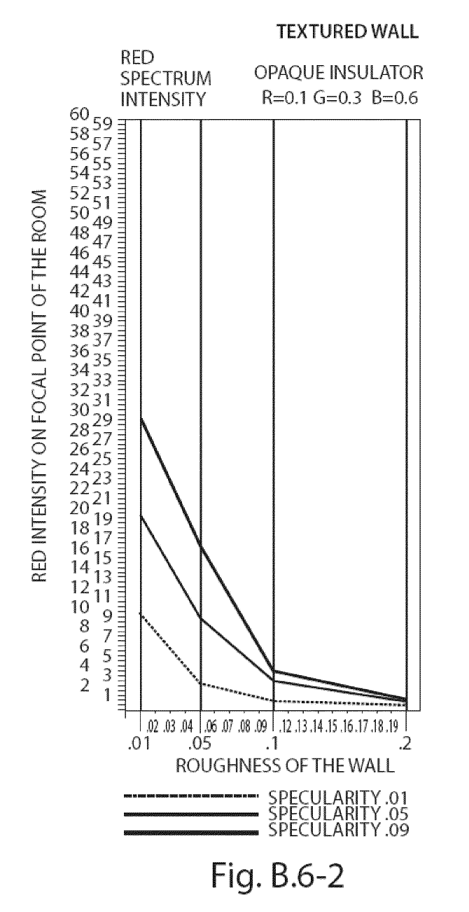

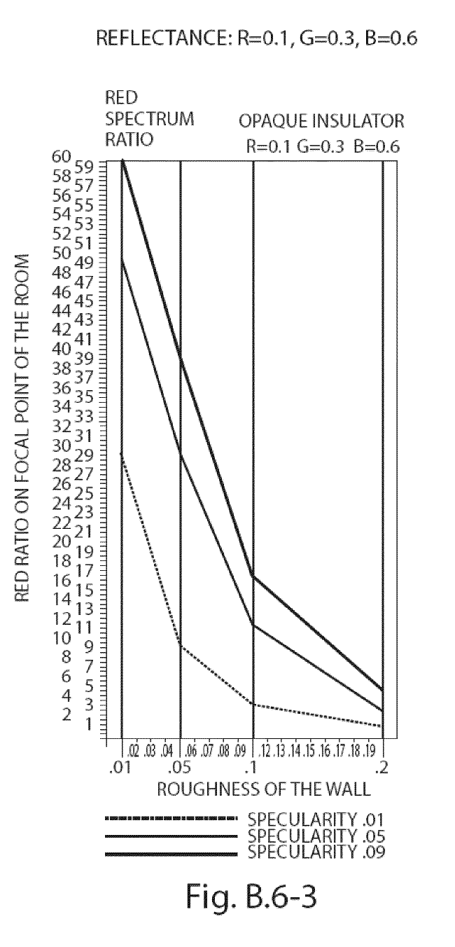

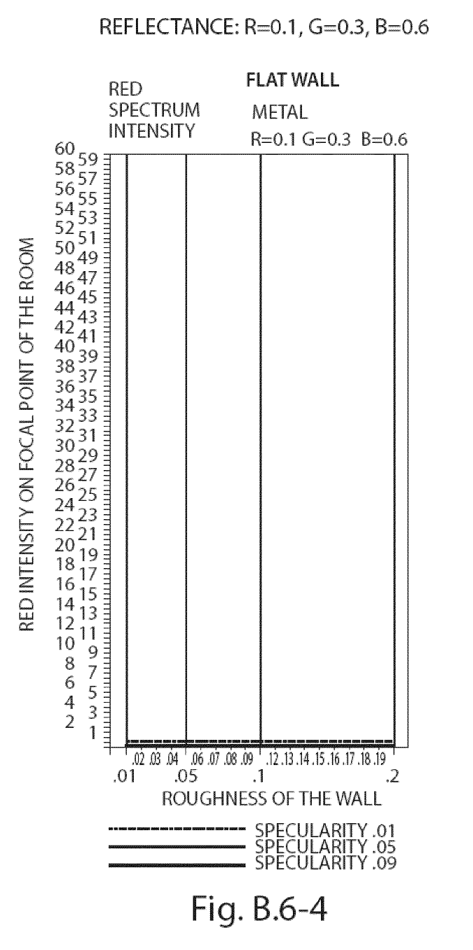

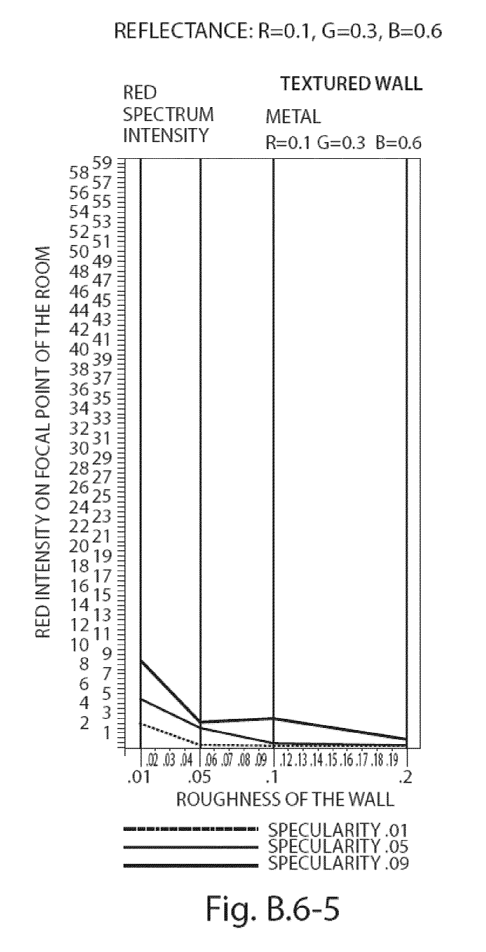

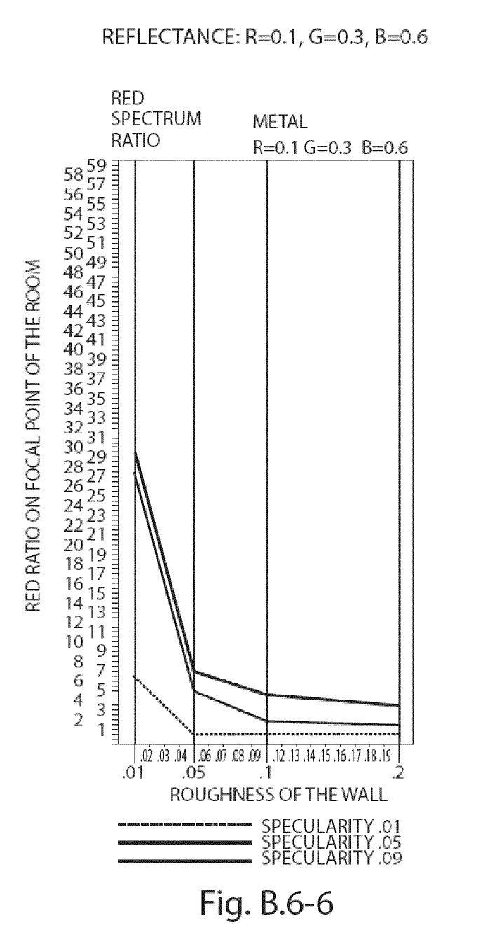

The simulations show that the blue part of the light spectrum is more enhanced by a non-metallic (insulator) material with a reflectance R=0.6, G=0.3, B=0.1 versus a metallic material with same reflectance. The reason for this result is that the specular reflection off a non-metallic surface gives spectral properties to the reflected rays that are similar to those of the original light source (the incident rays, which in this case are primarily blue). Further, in the case of the textured wall, the intensity is increased at the focal point because the texture of the wall concentrates the rays at that point. (see point 2 of FIG. 7.12A)

In the case of an insulator surface, when light is shone on a flat wall with a highly red reflectance (R=0.6, G=0.3, B=0.1), the spectral content of the light perceived by the eye at the focal point is mainly red and low in blue. The intensity level at the focal point is also low (see point 1 of FIG. 7.1.2A). When the textured wall is applied to the same case, the spectral content of the reflected light at the focal point is still rich in red, but much richer in the blue part of the spectrum as well. Further, the levels of intensity for both blue and red are much higher (see point 2 of FIG. 7.12A).

When the textured wall is viewed from a point other than the focal point, the intensity of the light and its blue spectral content decreases. The further one moves from the focal point, the lower the intensity and the blue spectral content. The reason for this phenomenon is the combination of spectral and diffuse reflection given by the surface texture. The part of the texture that redirects the reflected rays towards the focal point reflects light in a specular way, while the rest of the texture reflects light in a diffuse way. The light that has been reflected specularly from an insulator, acquires mostly the spectral characteristics of the incident light. The light that has been reflected diffusively from an insulator acquires mostly the spectral characteristics of the material.

The spectral and intensity characteristics of the reflected light from a metallic flat wall, at the focal point, do not differ much from those in the case of the insulator surface (see point 1 of FIG. 7.1.2B). However, intensity and spectrum at the focal point does differ from the insulator surface when the texture is applied. Spectral characteristics of light reflected specularly from a metal are modified by the color of the material. When the texture of the metallic wall reflects the rays towards the focal point, the spectrum of the light is enriched by the color of the metal (see point 2 and 4 of FIG. 7.1.2B).

In the case of a metal, when light is shone on a flat wall with a highly red reflectance (R=0.6, G=0.3, B=0.1), the spectral content of the light perceived by the eye at the focal point is mainly red and very low in blue. The intensity level at the focal point is also low (see point 1 of FIG. 7.1.2B). When the textured wall is applied to the same case, the spectral content of the reflected light at the focal point is still rich in red and not much richer in the blue (different form the case in an insulator. The level of intensity of the red is much higher (see point 2 of FIG. 7.12B).

Same as in the case of the insulator, the part of the texture that redirects the reflected rays towards the focal point reflects light in a specular way, while the rest of the texture reflects light in a diffuse way. The light that has been reflected specularly from a metal, acquires both the spectral characteristics of the incident light plus the spectral characteristics of the metal. Therefore, the difference between the intensity of the red in the flat wall case and the intensity of the red in the textured wall case is not very big.

The exemplification provided herein demonstrates that it is possible to concentrate and redirect certain parts of the light spectrum coming from a generic illumination system towards specific points in space by combining different light characteristics, optical properties of materials, and surface texture.

For example, light can be concentrated in certain areas depending on the point of view. In addition, specific parts of the spectrum of light can be redirected to specific points in space by using material properties and texture. Further, a surface with a specific reflectance may appear to have a different color and brightness depending on the angle it is viewed due to its texture an optical properties as well as the spectral and intensity properties of the light source.

From these simulations, it appears that insulator materials have great potential in achieving the above-stated goals because of their particular properties concerning specular reflection. In other words, insulators can be used to enhance specific parts of the visible spectrum of light. The variety of spectral and intensity combinations of light reflected off of insulators is higher than that of light reflected off of metals.

The exemplification provided should not be construed to exclude the use of different textures, textures at different scales, light sources with different spectral compositions, surfaces at different orientations towards the focal point, and different physical materials. Different combinations of these variables may change the intensity and spectral values in important ways, thus providing alternative ways to deliver proper light to the circadian system.

### DEFINITIONS

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

As used herein, “action spectra” is determined by comparing the number of photons required for the same biological effect at different wavelengths.

As used herein, “coherent light” refers to when the waves of the light beam have similar direction, amplitude, and phase that are capable of exhibiting interference.

As used herein, “electromagnetic spectrum” refers to a continuum electromagnetic energy, which is energy produced by electrocharges that is radiated as waves.

As used herein, “entrainment” is defined as the tendency for two oscillating bodies to lock into phase so that they vibrate in harmony. It is also defined as a synchronization of two or more rhythmic cycles.

As used herein, a “Fresnel lense” is a surface that brings parallel rays to a focal point after reflection; it is a parabolic shape, in three dimensions.

As used herein, “melatonin” is a hormone found in all living creatures that has a strong role in the sleep-wake cycles.

As used herein, “reflectance” refers to the percentage of light reflected from the object, which remains the same no matter what the illumination.

As used herein, “reflectivity” refers to an amount of light that is reflected from an object. It changes on the illumination; the more light level the more amount of light reflected and vice versa.

As used herein, “specularity” is the quality used in many 3D rendering programs to set the size and the brightness of a texture's reflection to light.

As used herein, “steradian” is defined as the solid angle subtended at the center of a sphere of radius r by a portion of the surface of the sphere having an area r².

As used herein, “transduction” is the transformation of one form of energy into another form of energy.

As used herein, “visible light” refers to the energy within the electromagnetic spectrum that humans can perceive and has wavelengths ranging from about 400 to 700 nanometers.

As used herein, “wavelength” refers to the distance between the peaks of the electromagnetic waves. They range from extremely short-wavelength gamma rays (about 10⁻¹² meters) to long-wavelength radio waves (10⁺⁴ meters).

### Exemplification

The invention will be more readily understood by reference to the following, which is included merely for purposes of illustration of certain aspects and embodiments of the present invention, and is not intended to limit the invention.

A light simulation of an indoor space conducted to study the spectral and intensity characteristics of a light stimulus modified by its interaction with a surface of varying material, color, specularity, roughness, and texture, was performed. RADIANCE was used as the software to simulate and measure light's interaction with different surfaces, and its resulting spectral and intensity characteristics at a specific point in the virtual room. The purpose of this simulation was to assess the possibility of different interactions of light and material surfaces to deliver a quality and quantity of light closer to that needed for the proper functioning of the circadian system.

Currently, there is no technique in architecture or lighting to assess the quality of indoor illumination relative to the circadian system. The simulation was designed by synthesizing key elements of the research discussed above: the study of indoor illumination; the eye as the sensory organ for the circadian system; light in the visible spectrum required for the circadian system; light characteristics to be manipulated architectonically (e.g. spectrum, intensity, and spatial distribution); radiometric units as the measure of light intensity; and materials to be studied (e.g. opaque insulators and metals).

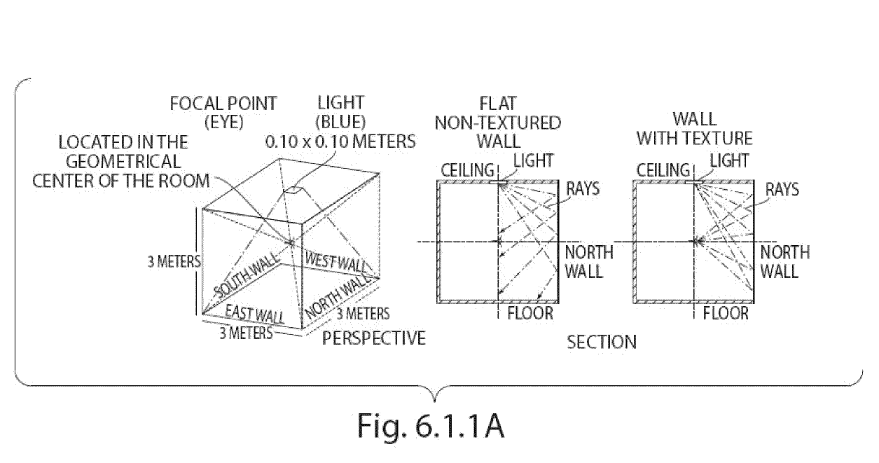

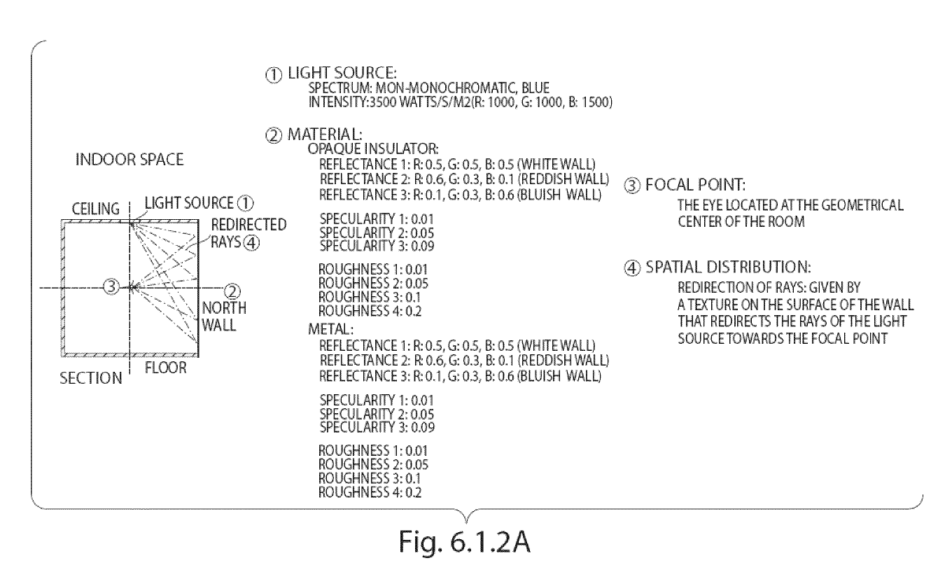

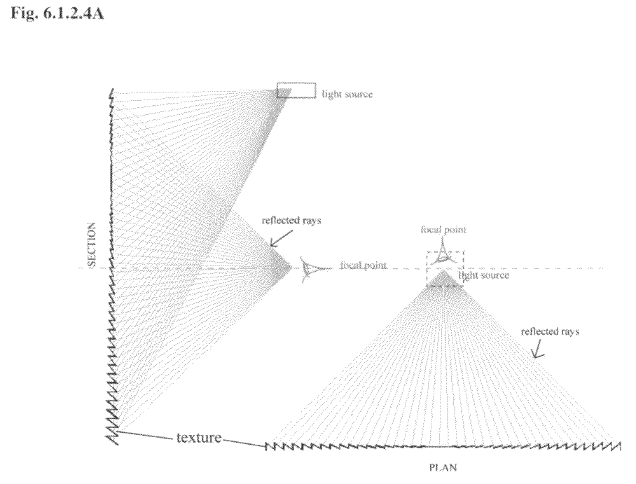

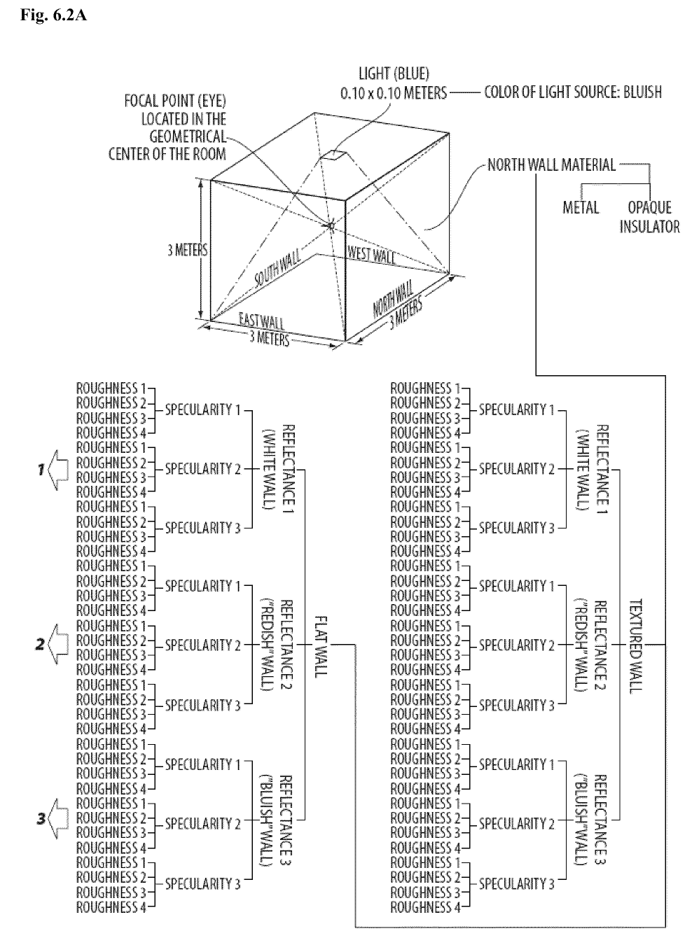

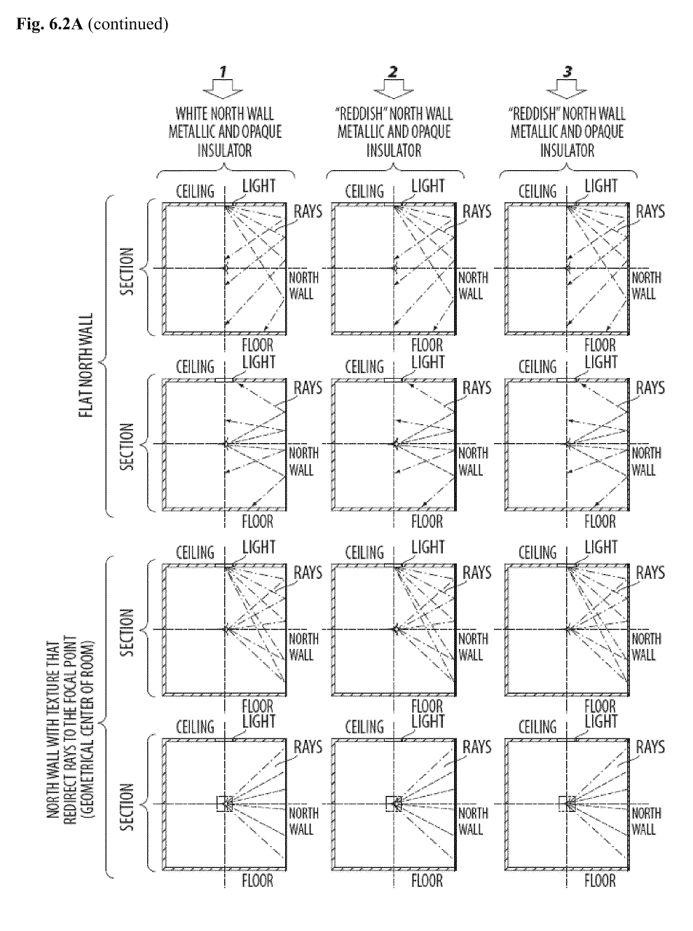

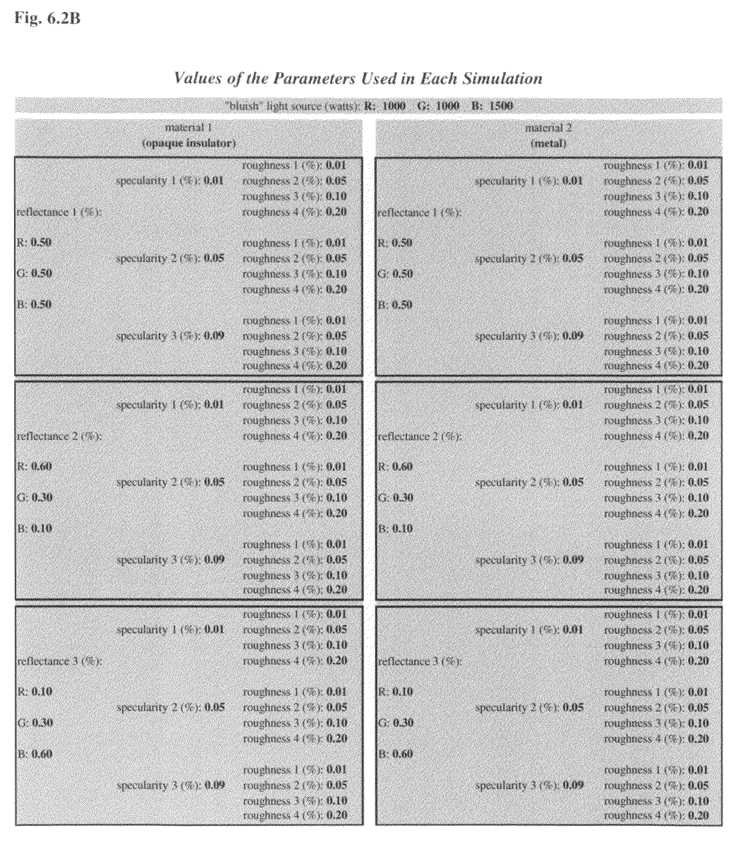

As discussed above, indoor illumination has been enhanced primarily for the visual system (visual clarity, luminance contrast, glare control, etc.) and not for stimulation of the circadian system. This deficiency in indoor lighting design leads to health consequences described as psycho-physical disorders. George Brainard and colleagues' laboratory experiment was described in part above: bright blue light was directed to the eye in order to stimulate the circadian photoreceptors; intensity and spectral characteristics of the light stimulus was specified; and a reflective material was used to reflect and redirect the rays of the light towards the focal point, in this case, the eye. FIG. 6A is a diagram that shows the parameters used in Brainard's experiment.

The eye is the perception organ for the light that stimulates the circadian system as well as the visual system. It is light within the visible spectrum that stimulates both systems; however, five main characteristic of light are needed in different qualities and quantities for the optimal stimulation of the visual system versus the circadian system. These five characteristics are: spectrum, intensity, duration, timing, and spatial distribution. From the architectural point of view, spectrum, intensity and spatial distribution are parameters that belong to design and that have to be taken into account when designing an interior space. It was explained above that radiometric units are adequate when measuring circadian light levels because circadian photoreceptors are still not completely understood.

The interaction between light and matter was described above. The difference between metal, insulators, and semiconductors was explained from the optical properties point of view. For the purpose of these studies, metals and opaque insulators were chosen for analysis in the simulation. Semiconductors were omitted because, generally speaking, their properties lie somewhere between those of metals and insulators. Additionally, semiconductors' atomic behavior is much more complex than that of metals and opaque insulators. Transparent insulators were also omitted because for the purpose of this invention transmission and refraction (optical properties corresponding to transparent materials) were not as relevant to the modification of light spectral and intensity characteristics.

The software RADIANCE was described above. Specifically how this program simulates and measures the intensity and spectrum of light as well as spectral and surface characteristics of materials. In RADIANCE, it is not possible to describe spectral characteristics in wavelength units. However, for these studies, description of spectral characteristics in red, green, and blue (the primary colors of light) is sufficient. RADIANCE was chosen as the simulation tool because of its capability to work with different colors of the visible light spectrum, and to provide measurements in radiometric units.

The design of the indoor space used in the RADIANCE simulations is based on the criteria mentioned above.

Two rooms, equal in dimension, were designed (see FIG. 6.1.1A). The dimension of the rooms is 3 m×3 m×3 m. A bluish diffuse light source, 0.10 m×0.10 m is located in the middle of the ceiling of each room. Each room is described as a cube with a black, non-textured, flat ceiling, floor, “south” wall, “west” wall, and “east” wall. These walls are black and without texture to avoid any alteration of the spectral and intensity characteristics of the source light. Only the material, color, and texture of the “north” wall vary in each simulation.

The difference between the two rooms lies in the “north” wall. In the first room, the “north” wall is completely flat, while in the second room, the north wall presents a texture that redirects the light rays of the light source that impact the “north” wall towards the geometrical center of the room. Therefore, the geometrical center of the room is the focal point representing the eye. The purpose of testing light's interaction with a flat, non-textured wall versus a textured wall was to compare the intensity and spatial distribution of light resulting at the focal point after the light source reflected off of each wall type.

Three main elements must be present in the simulated space: a light source, a surface with defined material properties, and a focal point (the eye). The spatial distribution of the light is proposed by varying the surface texture of the “north” wall (see FIG. 6.1.2A).

The light source emits with an RGB (red, green, and blue) spectral contribution of R=1000 w/s/m², G=1000 w/s/m², and B=1000 w/s/m². The spectral content is richer in blue and the intensity is given in radiance units.

As mentioned previously, only materials that behave like opaque insulators (defined by RADIANCE as plastics) and metals are studied in this simulation.

For both, three different reflectances were given in order to compare how different colors (defined by a material's reflectance) change the spectral and intensity characteristics of the reflected light. The three reflectances are the following: a reflectance balance of R=0.5, G=0.5, B=0.5, giving the appearance of a white wall; a high reflectance for red, and a pure reflectance for blue: R=0.6, G=0.3, R=0.1, giving the appearance of a “reddish-yellowish” wall; and a poor reflectance for red and high reflectance for blue: R=0.1, G=0.3, B=0.6, giving the appearance of a “bluish” wall.

Specularity defines a surface's level of reflectivity. As mentioned before, specular reflection shows differently in opaque insulators (defined as plastics in RADIANCE) versus metals. As mentioned above, highlights resulting form specular reflection acquire different spectral characteristics depending on whether the material is an opaque insulator or a metal. Highlights in opaque insulators present a spectral composition very similar to the spectral composition of the original light source, while highlights in metals present a spectral composition very close to the one of the metal. Specularity is a phenomenon that depends on the roughness of a surface, and the roughness of a surface is going to change the concentration of the reflected rays. In order to study the full range of materials in these categories (opaque insulators and metals), cases were analyzed with specularity in the range of 0.01-0.09 and surface roughness in the range of 0.01-0.20 for both materials.

The focal point, representing the eye, and located in the geometrical center of the room, is the point at which the spectral intensity of the reflected light was measured.

The spatial distribution of the rays was compared in the room with the flat, non-textured wall versus the room with the textured wall. In the second room, the texture of the north wall is designed to redirect all the rays that impact the wall towards the focal point (see FIG. 6.1.2.4A). The texture is composed by sections that reflect light specularly and others that reflect light diffusively. The sections that reflect light specularly also concentrate the rays towards the focal point. The textured was modeled in RADIANCE by varying the apparent local shape of the surface by perturbing the surface normal across the object surface as described above. This was done by using a mathematical description of these surface perturbations in order create the texture of the wall.

Figures B.1 to B.6 represent the variation of the blue and the red spectrum reflected by different reflectances in relationship with roughness and specularity changes. The first three graphs correspond to the intensity of the reflected blue part of the spectrum at the focal point and the last three graphs to the red part of the spectrum. For each figures: the three graphs on the top of the page correspond to the insulator wall, and the three graphs on the bottom of the page correspond to the metallic wall; the first row correspond to the flat wall, the second, to the textured wall, and the third, to the ratio of the flat wall versus the textured wall.

There are a total of 72 cases simulated for the room with the flat wall and 72 cases for the textured wall. For each material (metal and opaque insulator), the three reflectances were simulated; for each reflectance, three specularities were simulated; and for each specularity, four roughnesses were simulated (totaling 36 simulations for each material). See FIG. 6.2A and FIG. 6.2B.

Intensity of the blue and the red part of the spectrum, at the focal point of the room, was measured for all cases. Therefore, when referring to intensity levels it is understood that these correspond to measurements at the focal point. The green part of the spectrum is not mentioned taken into account that it is the blue and the red part of the spectrum that most influences the operation of the circadian system. The blue part of the visible spectrum has the most suppressive effects on the melatonin hormone, and “inform” the human biological clock that the environmental conditions correspond to light conditions. On the hand, the red part of the visible spectrum does not have any suppressive effects on the melatonin hormone, and “inform” the human biological clock that the environmental conditions correspond to dark conditions. Figure A.2-A.4 presents all the measurements taken for the 72 cases.

Figures A.2-A.4 show the intensity of the RGB components of the spectrum of the reflected wall at the focal point of the rooms (the one with the flat wall and the one with the textured wall). The second row correspond to the intensity of the red, green and blue component of the light reflected form the flat wall; the third row correspond to the intensity of the red, green and blue component of the light reflected from the textured wall; and the fourth row corresponds to the ratio between the intensity of the light reflected from the flat wall and the light reflected from the textured wall. Both materials, opaque insulator and metal, are compared. There are three sections corresponding to the three reflectances. Spectral composition of the light source (which does not vary), and the specularity and roughness of the surface of the walls are specified for each case.

Interestingly, the intensity of both the red and blue parts of the visible light spectrum is almost identical when reflected by either the flat wall (for both metal and insulator) or the textured wall (for both metal and insulator).

In all cases, intensity increases greatly when light is reflected off of the textured wall compared to the flat wall for both insulator and metal materials. Intensity increases from 2.5 times more (blue spectrum intensity reflected off the metallic wall with reflectance R=0.6, G=0.3, and B=0.1 and red spectrum intensity reflected off the metallic wall with reflectance R=0.1, G=0.3, and B=0.6) up to 40 times more (blue spectrum intensity reflected off the insulator wall with reflectance R=0.5, G=0.5, and B=0.5 and red spectrum intensity reflected off the insulator wall with reflectance R=0.5, G=0.5, and B=0.5) in comparison when reflected form a flat wall.

In all cases for the flat wall, including both insulators and metals of all reflectances, the intensity of the reflected light is almost the same when the materials are given maximum specularity and minimum roughness to when the materials are given minimum specularity and minimum roughness.

In all cases for the textured wall, including both insulators and metals of all reflectances, the intensity of the reflected light is highest when the materials are given maximum specularity and minimum roughness.

Inversely, in all cases for the textured wall, including both insulators and metals of all reflectances, the intensity of the reflected light is lowest when the materials are given minimum specularity and maximum roughness.

In all cases for the textured wall, including both insulators and metals of all reflectances, when roughness is given a constant, minimum value, intensity varies in relation to specularity.

In all cases for the textured wall, including both insulators and metals of all reflectances, when roughness is given a high value, different specularity levels do not change the resulting intensity.

The intensity of the blue part of the spectrum, is greatest when light is reflected off of a textured insulator wall of minimum roughness, maximum specularity, and a reflectance of R=0.5, G=0.5, B=0.5.

The second highest intensity of the blue part of the spectrum results when light is reflected off of a textured metal wall of minimum roughness, maximum specularity, and a reflectance of R=0.1, G=0.3, B=0.6.

Given a textured wall, the minimum intensity of the blue part of the spectrum results when light is reflected off of a metal wall of reflectance R=0.6, G=0.3, B=0.1.

The difference in intensity of the blue part of the spectrum reflected off of the flat versus textured wall (the ratio) is maximum when the wall is an insulator of reflectance R=0.6, G=0.3, B=0.1.

The difference in intensity of the blue part of the spectrum reflected off the flat versus textured wall (the ratio) is almost constant at all reflectances when the wall is a metal.

The intensity of the red part of the spectrum is greatest when light is reflected off of a textured insulator wall of minimum roughness, maximum specularity, and a reflectance of R=0.5, G=0.5, B=0.5.

The second highest intensity of the red part of the spectrum results when light is reflected off of a textured metal wall of minimum roughness, maximum specularity, and a reflectance of R=0.6, G=0.3, B=0.1.

Given a textured wall, the minimum intensity of the red part of the spectrum results when light is reflected off of a metal wall of reflectance R=0.1, G=0.3, B=0.6.

The difference in intensity of the red part of the spectrum reflected off of the flat versus textured wall (ratio) is maximum when the wall is an insulator of reflectance R=0.6, G=0.3, B=0.1.

The difference in intensity of the blue part of the spectrum reflected off the flat versus textured wall (the ratio) is almost constant at all reflectances when the wall is a metal.

### REFERENCES AND INCORPORATION BY REFERENCE

All of the U.S. patents and U.S. published patent applications cited herein are hereby incorporated by reference. In addition, the following references which are cited herein, by author and year of publication, are also hereby incorporated by reference.
Akerstedt, T., and Folkard, S. (1993). Sleep/Wake Regulation. In L. Wettengberg (Ed.), Light and Biological Rhythms in Man (pp. 237, 239, 240). Tarrytown, N.Y.: Pergamon Press.
Arendt, J. (1993). Some Effects of Light and Melatonin on Human Rythms. In L. Wettengberg (Ed.), Light and Biological Rhythms in Man (pp. 203-205, 208, 211-212). Tarrytown, N.Y.: Pergamon Press.
Bartsch, H., Bartsch, C., Mecke D., and Lippert T. H. (1993). The Relationship Between the Pineal Gland and Cancer: Seasonal Aspects. In L. Wettengberg (Ed.), Light and Biological Rhythms in Man (pp. 337-338). Tarrytown, N.Y.: Pergamon Press.
Beck-Friis, J. (1993) Seasonality and Seasonal Affective Disorders: Diagnostic Implications for the Outcome of Phototherapy. In L. Wettengberg (Ed.), Light and Biological Rhythms in Man (pp. 417). Tarrytown, N.Y.: Pergamon Press.
Brainard George, J. T. Hanfin, et al. (2001). Action Spectrum for Melatonin Regulation in Humans: Evidence for a Novel Circadian Photoreceptor. The Journal of Neuroscience, 21, 6405.
Brainard, G. C., Gaddy, J. R., Barker, F. M., Hanifin, J. P., and Rollag M. D. (1993) Mechanisms in the Eye that Mediate the Biological and Therapeutic Effects of Light in Humans. In L. Wettengberg (Ed.), Light and Biological Rhythms in Man (pp. 29-33, 41). Tarrytown, N.Y.: Pergamon Press.
Brainard, G. C., Hanfin, John P., Greeson, Jeffrey M., et. al. (2001). Action Spectrum for Melatonin Regulation in Humans: Evidence fro a Novel Circadian Photoreceptor. The Journal of Neuroscience, 21, 6405-6412.
Brainard, G. C., Kavet R., and Kheifets, L. I. (1999). The Relationship between Electromagnetic Field and Light Exposures to Melatonin and Breast Cancer Risk: A Review of the relative Literature. Journal of Pineal Research, 26, 65-100.
Comission Internationale de l'Ecleritage (CIE) (2004). Ocular Lighting Effects on Human Physiology and Behavior. Technical Report, CIE 158:2004.
Espiritu, R. C., Kripke, D. F., Ancoli-Israel, S., Mowen, M. A., Mason W. J., and Fell R. L. (1994). Low Illumination Experienced by San Diego Adults: Association with Atypical Depressive Symptoms. Biological Psychiatry, 35, 403-407.
Foster, R. G., Menaker, M. (1993). Circadian Photoreception in Mammals and Other Vertebrates. In L. Wettengberg (Ed.), Light and Biological Rhythms in Man (pp. 73, 81). Tarrytown, N.Y.: Pergamon Press.
Goldstein Bruce E. (2002). Sensation and Perception. Pacific Grove, Calif.: Wadsworth-Thomson Learning.
Gusowsky, Mary. (2000). Daylighting for Sustainable Design. New York: John Mc Graw Hill.
Hansen, J. (2001). Increased Breast Cancer Risk Among Women Who Work Predominantly at Night. Epidemology, 12, 74-77.
Hecht, E., and Zajac, A. (1974). Optics. London: Addison-Wesley.
Kirsch, R., Belgnaoui, S., Gourmelen, S., and Pevet, P. (1993). Daily Melatonin Infusion Entrains Free-Running Activity in Syrian and Siberian Hamsters. In L. Wettengberg (Ed.), Light and Biological Rhythms in Man (pp. 117). Tarrytown, N.Y.: Pergamon Press.
Kjellman, Bengt F., Thalen, Bjorn-Erik, and Wettenberg, Lennart. (1993). Light Treatment of Depressive States: Swedish Experiences at Latitude 59° North. In L. Wettengberg (Ed.), Light and Biological Rhythms in Man (pp. 352, 367). Tarrytown, N.Y.: Pergamon Press.
Klein, D. C. (1993). The Mammalian Melatonin Rhythm Generating System. In L. Wettengberg (Ed.), Light and Biological Rhythms in Man (pp. 55). Tarrytown, N.Y.: Pergamon Press.
Koller, M., Kundi, M., Stidl, H. G. T., and Haider, M. (1993). Personal Light Dosimetry in Permanent Night and Day Workers. Chronobiology International, 10, 143-155.
Komarov F. I., Rapoport S. I., Malinovskaya N. K., Neverovoa M. V., and Borilko V. A. (1993). Peptic Ulcer and Melatonin,” In L. Wettengberg (Ed.), Light and Biological Rhythms in Man (pp. 329). Tarrytown, N.Y.: Pergamon Press.
Kronauer, R. E., and Czeisler, C. A. (1993). Understanding the Use of Light to Control the Circadian Pacemaker in Humans. In L. Wettengberg (Ed.), Light and Biological Rhythms in Man (pp. 179, 218, 224, 233). Tarrytown, N.Y.: Pergamon Press.
Lacoste V. and Wetterberg L. (1993). Individual Variations of Rhythms in morning and Evening Types with Special Emphasis on Seasonal Difference. In L. Wettengberg (Ed.), Light and Biological Rhythms in Man (pp. 300). Tarrytown, N.Y.: Pergamon Press.
Lavie P. (2001). Sleep-wake as a Biological Rhythm. Annual Review of Psychology, 52, 277-303.
Lewy A. J. and Sack R. L. (1993). The Use of Melatonin as a Marker for Circadian Phase and as a Chronobiotic in Blind Sighted Humans. In L. Wettengberg (Ed.), Light and Biological Rhythms in Man (pp. 174, 178-180). Tarrytown, N.Y.: Pergamon Press.
Rea Mark S. (2002). Light—Much More than Vision. In Rensselaer Poltechnic Institute. Proceedings from the 5th Lighting Research Office, Lighting Research Symposium. Troy, N.Y.
Rea Mark S. (2002). Light—Much More than Vision. In Rensselaer Poltechnic Institute. Proceedings from the 5th Lighting Research Office, Lighting Research Symposium. Troy, N.Y.
Reiter R. J. (1993). The Mammalian Pineal Gland as an End Organ of the Visual System. In L. Wettengberg (Ed.), Light and Biological Rhythms in Man (pp. 149. 352, 367). Tarrytown, N.Y.: Pergamon Press.
Tilley, R. (2000). Colour and the Optical Properties of Materials. New York: John Wiley & Sons.
Touitou Y. and Haus E. (1993). Circannual Variation of Some Endocrine and Neuroendocrine Functions in Humans. In L. Wettengberg (Ed.), Light and Biological Rhythms in Man (pp. 318). Tarrytown, N.Y.: Pergamon Press.
Ward Larson, Greg, and Shakespeare, Rob. (1998). Rendering With Radiance: The Art And Science Of Lighting Visualization. California: Space and Light.
Washington, D.C.: United States Environmental Protection Agency, Office of Air and Radiation (2001). Why Study human Health Indoors? In Healthy Buildings, Healthy People: A Vision for the 21^{st} Century. http://www.epa.gov (cited 5 May 2006).
Waterhouse J., Costa G., Hamma M., Knauth P., Minors D., Sitka U., and Weinert D. (1993). Overview: Unmasking Temperature Data Obtained in Field Conditions. In L.
Wettengberg (Ed.), Light and Biological Rhythms in Man (pp. 189). Tarrytown, N.Y.: Pergamon Press.
Wettenberg L, Eberhard G., et. al. (1993). The Influence of Age, Sex, Height, Weight, Urine Volume and Latitude on Melatonin Concentrations in Urine from Normal Subjects: A Multinational Study. In L. Wettengberg (Ed.), Light and Biological Rhythms in Man (pp. 276, 3285). Tarrytown, N.Y.: Pergamon Press.
Wibom R. (1993). Light-Definitions and Measurements. In L. Wettengberg (Ed.), Light and Biological Rhythms in Man (pp. 26). Tarrytown, N.Y.: Pergamon Press.
Wirz-Justice A., Graw P., Krauchi K., et al. (1993). Effect of Light on Unmasked Circadian Rhythms in Winter Depression. In L. Wettengberg (Ed.), Light and Biological Rhythms in Man (pp. 385). Tarrytown, N.Y.: Pergamon Press.

### EQUIVALENTS

Those skilled in the art will recognize, or be able to ascertain using no more than routine experimentation, many equivalents to the specific embodiments of the invention described herein. Such equivalents are intended to be encompassed by the following claims.

I claim:

1. A method of stimulating the circadian system of a subject comprising the step of reflecting light off of a surface towards the subject.

2. The method of claim 1, wherein said light is non-monochromatic.

3. The method of claim 1, wherein said light comprises blue light.

4. The method of claim 1, wherein said light comprises light having a wavelength between about 460 nm and about 480 nm.

5. The method of claim 1, wherein the radiant intensity of the light is between about 2,000 W/sr and about 5,000 W/sr.

6. The method of claim 1, wherein the radiant intensity of the light is between about 3,000 W/sr and about 4,000 W/sr.

7. The method of claim 1, wherein the radiant intensity of the light is about 3,500 W/sr.

8. The method of claim 7, wherein the red radiant intensity is about 1,000 W/sr; the green radiant intensity is about 1,000 W/sr; and the blue radiant intensity is about 1,500 W/sr.

9. The method of claim 1, wherein the RGB reflectance of the surface is about 0.1 (red), about 0.3 (green) and about 0.6 (blue).

10. The method of claim 1, wherein the RGB reflectance of the surface is about 0.5 (red), about 0.5 (green) and about 0.5 (blue).

11. The method of claim 1, wherein the RGB reflectance of the surface is about 0.6 (red), about 0.3 (green) and about 0.1 (blue).

12. The method of claim 1, wherein the specularity of the surface is between about 0.01 and about 0.09.

13. The method of claim 1, where the roughness of the surface is between about 0.01 and 0.20.

14. The method of claim 1, wherein the surface comprises a non-metallic opaque insulator.

15. The method of claim 1, wherein the surface comprises a plastic.

16. The method of claim 1, wherein the surface comprises a metal.

17. The method of claim 1, wherein the surface is substantially flat.

18. The method of claim 1, wherein the surface is curved.

19. The method of claim 1, wherein the surface is part of a wall.

20. The method of claim 1, wherein the stimulation leads to improved performance, alertness, mood, or sense of well being of a subject.
